(19) United States
(12) Patent Application Publication
(10) Pub. No.: US 2012/0115912 A1
(43) Pub. Date: May 10, 2012

(54) RXR AGONIST COMPOUNDS AND METHODS

(76) Inventors: Gary E. Landreth, Shaker Heights, OH (US); Paige E. Cramer, Cleveland Heights, OH (US)

(21) Appl. No.: 13/383,131

(22) PCT Filed: Jul. 12, 2010
(86) PCT No.: PCT/US10/41707

§ 371 Date: Jan. 9, 2012

Related U.S. Application Data
(60) Provisional application No. 61/224,709, filed on Jul. 10, 2009.

(51) Int. Cl. A61K 31/4439; A61K 31/426; A61K 31/41; A61P 25/16; A61K 31/423; A61K 31/192; A61P 25/00; A61P 25/28; A61K 31/427; A61K 31/422

(52) U.S. Cl. 514/342; 514/369; 514/360; 514/376; 514/375; 514/568

(57) ABSTRACT

A method of treating a PPARγ and/or RXR related disease or disorder in a subject includes adminstering to the subject an RXR agonist alone or in combination with a PPARγ agonist.

### RELATED APPLICATION

This application claims priority from U.S. Provisional Application No. 60/224,709, filed Jul. 10, 2009, the subject matter which is incorporated herein by reference.

### FIELD OF THE INVENTION

The present invention relates to methods and compositions for treating Alzheimer's disease and other diseases and conditions with an inflammatory component (e.g., central nervous system injury, dermatological disorder, cystic fibrosis). In particular, the present invention provides agents that regulate the production of proinflammatory and/or neurotoxic products involved in Alzheimer's disease and other inflammatory diseases.

### BACKGROUND OF THE INVENTION

Alzheimer's disease (AD) is a complex multi-genic neurodegenerative disorder characterized by progressive impairments in memory, behavior, language, and visuo-spatial skills, ending ultimately in death. Hallmark pathologies within vulnerable regions include extracellular β-amyloid deposits, intracellular neurofibrillary tangles, synaptic loss, and extensive neuronal cell death. Research on the causes and treatments of Alzheimer's disease has led investigators down numerous avenues. Although many models have been proposed, no single model of AD satisfactorily accounts for all neuropathologic findings as well as the requirement of aging for disease onset. The mechanisms of disease progression are equally unclear. Considerable human genetic evidence has implicated alterations in production or processing of the human amyloid precursor protein (APP) in the etiology of the disease. However, intensive research has proven that AD is a multifactorial disease with many different, perhaps overlapping, etiologies.

To date, Alzheimer's disease is the third most expensive disease in the United States, costing society approximately $100 billion each year. It is one of the most prevalent illnesses in the elderly population, and with the aging of society, will become even more significant. Costs associated with AD include direct medical costs such as nursing home care, direct nonmedical costs such as in-home day care, and indirect costs such as lost patient and care giver productivity. Medical treatment may have economic benefits by slowing the rate of cognitive decline, delaying institutionalization, reducing care giver hours, and improving quality of life. Pharmacoeconomic evaluations have shown positive results regarding the effect of drug therapy on nursing home placement, cognition, and care giver time.

Thus far, the therapeutic strategies attempted have targeted neurotransmitter replacement, or the preservation of normal brain structures, which potentially provide short-time relief, but do not prevent neuronal degeneration and death. Thus, there is a need for therapies that prevent neuronal degeneration and death associated with Alzheimer's disease and provide long-term relief.

### SUMMARY OF THE INVENTION

One aspect of the present invention relates to a method of treating Alzheimer's disease in a subject. The method includes administering to the subject a therapeutically effective amount of at least one RXR agonist. In one example, the RXR agonist can include Bexarotene.

In another aspect, the method of treating Alzheimer's disease can include administering a PPARγ agonist in combination with the RXR agonist. The PPARγ agonist can include a thiazolidinedione or a derivative thereof. The thiazolidinedione can include at least one compound or a pharmaceutically acceptable salt thereof selected from the group consisting of: (+)-5[[4-[(3,4-dihydro-6-hydroxy-2,5,7,8-tetramethyl-2H-1-benzopyran-2-yl)methoxy]phenyl]methyl]-2,4thiazolidinedione; 5-[4-[2-(5-ethylpyridin-2-yl)ethoxyl]benzyl]thiazolidine-2,4-dione; 5-[4-[(1-methylcyclohexyl)methoxy]benzyl]thiazolidine-2,4-dione; (ciglitazone); 4-(2-naphthylmethyl)-1,2,3,5-oxathiadiazole-2-oxide; 5-[4-[2-[(N-(benzoxazol-2-yl)-N-methylamino]ethoxy]benzyl]-5-methlthiazolidine-2,4-dione; 5-[4-[2-[2,4dioxo-5-phenylthiazolidine-3-yl)ethoxy]benzyl]thiazolidine-2,4-dione; 5-[4-[2-[(N-methyl-N-(phenoxycarbonyl)amino]ethoxy]benzyl]thiazolidine-2,4-dione; 5-[4-[2-phenoxyethoxy)benzyl]thiazolidine-2,4-dione; 5-[4-[2-(4-chorophenyl)ethylsulfonyl]benzyl]thiazolidine-2,4-dione; 5-[4-[3-(5-methyl-2-phenyloxazol-4-yl)propionyl]benzyl]thiazolidine-2,4-dione; 5-[[4-(3-hydroxy-1-methylcyclohexyl)methoxy]benzyl]thiazolidine-2,4-dione; 5-[4-[2-(5-methyl-2-phenyloxazol-4-yl)ethoxyl]benzyl]thiazolidine-2,4-dione; 5-[(2-benzyl-2,3-dihydrobenzopyran)-5-ylmethyl]thiazolidine-2,4-dione; 5-[[2-(2-naphthylmethyl)benzoxazol]-5-ylmethyl]thiazolidine-2,4-dione; 5-[4-[2-(3-phenylureido)ethoxyl]benzyl]thiazolidine-2,4-dione; 5-[4-[2-(N-benzoxazol-2-yl)-N-metholamino]ethoxy]benzyl]thiazolidine-2,4-dione; 5-[4-[3-(5-methyl-2-phenyloxazol-4-yl)propionyl]benzyl]thiazolidine-2,4-dione; 5-[2-(5-methyl-2-phenyloxazol-4-ylmethyl)benzofuran-5-ylmethyl]oxazolidine-2,4-dione; 5-[4-[2-(N-methyl-N-(2-pyridyl)amino]ethoxy]benzyl]thiazolidine-2,4-dione; and 5-[4-[2-(N-(benzoxazol-2-yl)-N-methylamino]ethoxy]benzyl]oxazolidine-2,4-dione.

In a further aspect, the method of treating Alzheimer's disease can include administering a LXR agonist in combination with the RXR agonist and the PPARγ agonist.

Another aspect of the present invention relates to a method of treating a central nervous system injury in a subject. The method includes administering to the subject a therapeutically effective amount of at least one RXR agonist. In one example, the RXR agonist can include Bexarotene.

In another aspect, the method of treating a central nervous system injury can include administering a PPARγ agonist in combination with the RXR agonist. The PPARγ agonist can include a thiazolidinedione or a derivative thereof. The thiazolidinedione can include at least one compound or a pharmaceutically acceptable salt thereof selected from the group consisting of: (+)-5[[4-[(3,4-dihydro-6-hydroxy-2,5,7,8-tetramethyl-2H-1-benzopyran-2-yl)methoxy]phenyl]methyl]-2,4thiazolidinedione; 5-[4-[2-(5-ethylpyridin-2-yl)ethoxyl]benzyl]thiazolidine-2,4-dione; 5-[4-[(1-methylcyclohexyl)methoxy]benzyl]thiazolidine-2,4-dione; (ciglitazone); 4-(2-naphthylmethyl)-1,2,3,5-oxathiadiazole-2-oxide; 5-[4-[2-[(N-(benzoxazol-2-yl)-N-methylamino]ethoxy]benzyl]-5-methlthiazolidine-2,4-dione; 5-[4-[2-[2,4dioxo-5-phenylthiazolidine-3-yl)ethoxy]benzyl]thiazolidine-2,4-dione; 5-[4-[2-[(N-methyl-N-(phenoxycarbonyl)amino]ethoxy]benzyl]thiazolidine-2,4-dione; 5-[4-[2-phenoxyethoxy)benzyl]thiazolidine-2,4-dione; 5-[4-[2-(4-chorophenyl)ethylsulfonyl]benzyl]thiazolidine-2,4-dione; 5-[4-[3-(5-methyl-2-phenyloxazol-4-yl)propionyl]benzyl]thiazolidine-2,4-dione; 5-[[4-(3-hydroxy-1-methylcyclohexyl)methoxy]benzyl]thiazolidine-2,4-dione; 5-[4-[2-(5-methyl-2-phenyloxazol-4-yl)ethoxyl]benzyl]thiazolidine-2,4-dione; 5-[(2-benzyl-2,3-dihydrobenzopyran)-5-ylmethyl]thiazolidine-2,4-dione; 5-[[2-(2-naphthylmethyl)benzoxazol]-5-ylmethyl]thiazolidine-2,4-dione; 5-[4-[2-(3-phenylureido)ethoxyl]benzyl]thiazolidine-2,4-dione; 5-[4-[2-(N-benzoxazol-2-yl)-N-metholamino]ethoxy]benzyl]thiazolidine-2,4-dione; 5-[4-[3-(5-methyl-2-phenyloxazol-4-yl)propionyl]benzyl]thiazolidine-2,4-dione; 5-[2-(5-methyl-2-phenyloxazol-4-ylmethyl)benzofuran-5-ylmethyl]oxazolidine-2,4-dione; 5-[4-[2-(N-methyl-N-(2-pyridyl)amino]ethoxy]benzyl]thiazolidine-2,4-dione; and 5-[4-[2-(N-(benzoxazol-2-yl)-N-methylamino]ethoxy]benzyl]oxazolidine-2,4-dione.

In a further aspect, the method of treating a neural inflammation can include administering a LXR agonist in combination with the RXR agonist and the PPARγ agonist.

Another aspect of the present invention relates to a method of treating neural inflammation in a subject. The method includes administering to the subject a therapeutically effective amount of at least one RXR agonist. In one example, the RXR agonist can include Bexarotene.

In another aspect, the method of treating neural inflammation can include administering a PPARγ agonist in combination with the RXR agonist. The PPARγ agonist can include a thiazolidinedione or a derivative thereof. The thiazolidinedione can include at least one compound or a pharmaceutically acceptable salt thereof selected from the group consisting of: (+)-5[[4-[(3,4-dihydro-6-hydroxy-2,5,7,8-tetramethyl-2H-1-benzopyran-2-yl)methoxy]phenyl]methyl]-2,4thiazolidinedione; 5-[4-[2-(5-ethylpyridin-2-yl)ethoxyl]benzyl]thiazolidine-2,4-dione; 5-[4-[(1-methylcyclohexyl)methoxy]benzyl]thiazolidine-2,4-dione; (ciglitazone); 4-(2-naphthylmethyl)-1,2,3,5-oxathiadiazole-2-oxide; 5-[4-[2-[(N-(benzoxazol-2-yl)-N-methylamino]ethoxy]benzyl]-5-methlthiazolidine-2,4-dione; 5-[4-[2-[2,4dioxo-5-phenylthiazolidine-3-yl)ethoxy]benzyl]thiazolidine-2,4-dione; 5-[4-[2-[(N-methyl-N-(phenoxycarbonyl)amino]ethoxy]benzyl]thiazolidine-2,4-dione; 5-[4-[2-phenoxyethoxy)benzyl]thiazolidine-2,4-dione; 5-[4-[2-(4-chorophenyl)ethylsulfonyl]benzyl]thiazolidine-2,4-dione; 5-[4-[3-(5-methyl-2-phenyloxazol-4-yl)propionyl]benzyl]thiazolidine-2,4-dione; 5-[[4-(3-hydroxy-1-methylcyclohexyl)methoxy]benzyl]thiazolidine-2,4-dione; 5-[4-[2-(5-methyl-2-phenyloxazol-4-yl)ethoxyl]benzyl]thiazolidine-2,4-dione; 5-[(2-benzyl-2,3-dihydrobenzopyran)-5-ylmethyl]thiazolidine-2,4-dione; 5-[[2-(2-naphthylmethyl)benzoxazol]-5-ylmethyl]thiazolidine-2,4-dione; 5-[4-[2-(3-phenylureido)ethoxyl]benzyl]thiazolidine-2,4-dione; 5-[4-[2-(N-benzoxazol-2-yl)-N-metholamino]ethoxy]benzyl]thiazolidine-2,4-dione; 5-[4-[3-(5-methyl-2-phenyloxazol-4-yl)propionyl]benzyl]thiazolidine-2,4-dione; 5-[2-(5-methyl-2-phenyloxazol-4-ylmethyl)benzofuran-5-ylmethyl]oxazolidine-2,4-dione; 5-[4-[2-(N-methyl-N-(2-pyridyl)amino]ethoxy]benzyl]thiazolidine-2,4-dione; and 5-[4-[2-(N-(benzoxazol-2-yl)-N-methylamino]ethoxy]benzyl]oxazolidine-2,4-dione.

In a further aspect, the method of treating neural inflammation can include administering a LXR agonist in combination with the RXR agonist and the PPARγ agonist.

Yet another aspect of the present invention relates to a method of treating cystic fibrosis in a subject. The method includes administering to the subject a therapeutically effective amount of at least one RXR agonist. In one example, the RXR agonist can include Bexarotene.

In another aspect, the method of treating cystic fibrosis can include administering a PPARγ agonist in combination with the RXR agonist. The PPARγ agonist can include a thiazolidinedione or a derivative thereof. The thiazolidinedione can include at least one compound or a pharmaceutically acceptable salt thereof selected from the group consisting of: (+)-5[[4-[(3,4-dihydro-6-hydroxy-2,5,7,8-tetramethyl-2H-1-benzopyran-2-yl)methoxy]phenyl]methyl]-2,4thiazolidinedione; 5-[4-[2-(5-ethylpyridin-2-yl)ethoxyl]benzyl]thiazolidine-2,4-dione; 5-[4-[(1-methylcyclohexyl)methoxy]benzyl]thiazolidine-2,4-dione; (ciglitazone); 4-(2-naphthylmethyl)-1,2,3,5-oxathiadiazole-2-oxide; 5-[4-[2-[(N-(benzoxazol-2-yl)-N-methylamino]ethoxy]benzyl]-5-methlthiazolidine-2,4-dione; 5-[4-[2-[2,4dioxo-5-phenylthiazolidine-3-yl)ethoxy]benzyl]thiazolidine-2,4-dione; 5-[4-[2-[(N-methyl-N-(phenoxycarbonyl)amino]ethoxy]benzyl]thiazolidine-2,4-dione; 5-[4-[2-phenoxyethoxy)benzyl]thiazolidine-2,4-dione; 5-[4-[2-(4-chorophenyl)ethylsulfonyl]benzyl]thiazolidine-2,4-dione; 5-[4-[3-(5-methyl-2-phenyloxazol-4-yl)propionyl]benzyl]thiazolidine-2,4-dione; 5-[[4-(3-hydroxy-1-methylcyclohexyl)methoxy]benzyl]thiazolidine-2,4-dione; 5-[4-[2-(5-methyl-2-phenyloxazol-4-yl)ethoxyl]benzyl]thiazolidine-2,4-dione; 5-[(2-benzyl-2,3-dihydrobenzopyran)-5-ylmethyl]thiazolidine-2,4-dione; 5-[[2-(2-naphthylmethyl)benzoxazol]-5-ylmethyl]thiazolidine-2,4-dione; 5-[4-[2-(3-phenylureido)ethoxyl]benzyl]thiazolidine-2,4-dione; 5-[4-[2-(N-benzoxazol-2-yl)-N-metholamino]ethoxy]benzyl]thiazolidine-2,4-dione; 5-[4-[3-(5-methyl-2-phenyloxazol-4-yl)propionyl]benzyl]thiazolidine-2,4-dione; 5-[2-(5-methyl-2-phenyloxazol-4-ylmethyl]benzofuran-5-ylmethyl]oxazolidine-2,4-dione; 5-[4-[2-(N-methyl-N-(2-pyridyl)amino]ethoxy]benzyl]thiazolidine-2,4-dione; and 5-[4-[2-(N-(benzoxazol-2-yl)-N-methylamino]ethoxy]benzyl]oxazolidine-2,4-dione.

In a further aspect, the method of treating cystic fibrosis can include administering a LXR agonist in combination with the RXR agonist and the PPARγ agonist.

Another aspect of the present invention relates to a method of treating a dermatological disorder in a subject. The method includes administering to the subject a therapeutically effective amount of at least one RXR agonist. In one example, the RXR agonist can include Bexarotene.

In another aspect, the method of treating a dermatological disorder can include administering a PPARγ agonist in combination with the RXR agonist. The PPARγ agonist can include a thiazolidinedione or a derivative thereof. The thiazolidinedione can include at least one compound or a pharmaceutically acceptable salt thereof selected from the group consisting of: (+)-5[[4-[(3,4-dihydro-6-hydroxy-2,5,7,8-tetramethyl-2H-1-benzopyran-2-yl)methoxy]phenyl]methyl]-2,4thiazolidinedione; 5-[4-[2-(5-ethylpyridin-2-yl)ethoxyl]benzyl]thiazolidine-2,4-dione; 5-[4-[(1-methylcyclohexyl)methoxy]benzyl]thiazolidine-2,4-dione; (ciglitazone); 4-(2-naphthylmethyl)-1,2,3,5-oxathiadiazole-2-oxide; 5-[4-[2-[(N-(benzoxazol-2-yl)-N-methylamino]ethoxy]benzyl]-5-methlthiazolidine-2,4-dione; 5-[4-[2-[2,4dioxo-5-phenylthiazolidine-3-yl)ethoxy]benzyl]thiazolidine-2,4-dione; 5-[4-[2-[(N-methyl-N-(phenoxycarbonyl)amino]ethoxy]benzyl]thiazolidine-2,4-dione; 5-[4-[2-phenoxyethoxy)benzyl]thiazolidine-2,4-dione; 5-[4-[2-(4-chorophenyl)ethylsulfonyl]benzyl]thiazolidine-2,4-dione; 5-[4-[3-(5-methyl-2-phenyloxazol-4-yl)propionyl]benzyl]thiazolidine-2,4-dione; 5-[[4-(3-hydroxy-1-methylcyclohexyl)methoxy]benzyl]thiazolidine-2,4-dione; 5-[4-[2-(5-methyl-2-phenyloxazol-4-yl)ethoxyl]benzyl]thiazolidine-2,4-dione; 5-[(2-benzyl-2,3-dihydrobenzopyran)-5-ylmethyl]thiazolidine-2,4-dione; 5-[[2-(2-naphthylmethyl)benzoxazol]-5-ylmethyl]thiazolidine-2,4-dione; 5-[4-[2-(3-phenylureido)ethoxyl]benzyl]thiazolidine-2,4-dione; 5-[4-[2-(N-benzoxazol-2-yl)-N-metholamino]ethoxy]benzyl]thiazolidine-2,4-dione; 5-[4-[3-(5-methyl-2-phenyloxazol-4-yl)propionyl]benzyl]thiazolidine-2,4-dione; 5-[2-(5-methyl-2-phenyloxazol-4-ylmethyl)benzofuran-5-ylmethyl]oxazolidine-2,4-dione; 5-[4-[2-(N-methyl-N-(2-pyridyl)amino]ethoxy]benzyl]thiazolidine-2,4-dione; and 5-[4-[2-(N-(benzoxazol-2-yl)-N-methylamino]ethoxy]benzyl]oxazolidine-2,4-dione.

In a further aspect, the method of treating a dermatological disorder can include administering a LXR agonist in combination with the RXR agonist and the PPARγ agonist.

### DESCRIPTION OF DRAWINGS

FIG. 1 is a schematic diagram illustrating the regulation of lipid metabolism by nuclear receptors.

FIG. 2 is a schematic diagram illustrating the RXR agonist Bexarotene's ability to induce the expression of LXR target genes, ABCA1 and ApoE and promoting Aβ degradation.

FIG. 3 illustrates an immunoassay and graphs showing RXR activation drives expression of LXR target genes. Primary microglia were treated with increasing concentrations of Bexarotene for 24 hours. Cell lysates were subjected to Western analysis for ABCA1, ABCG1, ApoE, and GAPDH as a loading control.

FIG. 4 illustrates an immunoassay showing ApoE lipidation status is increased after RXR agonist treatment. Primary astrocytes were treated with increasing concentrations of Bexarotene for 48 hours. Conditioned media was subjected to native gel electrophoresis followed by Western analysis for ApoE.

FIG. 5 illustrates graphs showing RXR agonists stimulate Aβ degradation. A. Primary microglia were treated with the RXR agonist, 9cisRA or B. Bexarotene for 24 hours followed by 18 hours with 2 μg/mL soluble Aβ.

FIG. 6 illustrates graphs showing Bexarotene crosses the blood brain barrier (BBB) and drives gene expression. Four mice (2 transgenic APP/PS1 and 2 WT littermates) were orally gavaged with 100 mg/kg Bexarotene or vehicle for 7 days. Gene expression in brain homogenates was monitored by Western analysis for ABCA1, ABCG1, ApoE and GAPDH as a loading control.

FIG. 7 illustrates graphs showing oral RXR agonist treatment reduces both soluble and insoluble Aβ1-40 and 1-42. ELISA data of serial extractions of brain homogenates of AD mouse models (n≧4) orally gavaged for 7 days with 100 mg/kg/day of Bexarotene or vehicle (water). Soluble Aβ was extracted using diethylamine, followed by a formic acid extraction to yield the insoluble Aβ. Detection antibodies directed at either Aβ-40 or 1-42 were used to determine the reduction of different species of Aβ.

FIG. 8 illustrates photographs and a graph showing oral RXR agonist treatment reduces plaque burden in an AD mouse model. Cryostat sections of brain (10 um) stained for 6E10, marking amyloid beta and amyloid precursor protein. Animals were orally gavaged for 7 days with 100 mg/kg/day of Bexarotene (B) or vehicle (A). Five animals per treatment were analyzed. 6 sections per animal throughout the brain from about 10 μm prior to about 100 μm beyond the hippocampus and at least 4 images of the cortex were analyzed per section for plaque area (C). (10×)

FIG. 9 illustrates oral RXR agonist treatment improves the behavior of an AD animal model. 6 month old transgenic positive (Tg pos) AD animals were orally gavaged for 7 days with Bexarotene (100 mg/kg/day) or water. Wildtype littermates were used as a control and were orally gavaged with water for 7 days (n=5). Following treatment, animals underwent contextual fear conditioning. The number of times the animals froze over a two minute period was assessed after training.

FIG. 10 illustrates an immunoassay and graphs showing RXR activation of primary astrocytes drives expression of LXR target genes. Primary astrocytes were treated with increasing concentrations of Bexarotene for 24 hours. Cell lysates were subjected to Western analysis for ABCA1, ABCG1 and ApoE. Actin served as a load control.

FIG. 11 illustrates a graph showing RXR activation drives expression of PPARγ target gene, CD36. Primary murine astrocytes were treated with 10 nM Bexarotene for a defined time. Cell lysates were subjected to quantitative RT-PCR. GAPDH served as a control.

FIG. 12 illustrates a graph showing RXR agonist stimulate Aβ degradation in astrocytes. Primary astrocytes were treated with Bexarotene for 24 hours followed by 18 hours with 2 ug/mL soluble Aβ.

FIG. 13 illustrates graphs showing degradation by RXR agonist requires ApoE. Murine ApoE knock out microglia (A) and astrocytes (B) treated with Bexarotene and/or exogenous 1 μg/mL ApoE for 24 hours followed by 18 hours with 2 μg/mL soluble Aβ and drug.

FIG. 14 illustrates graphs showing RXR mediated intracellular Aβ degradation is prevented by inhibiting PPARγ or LXR. Microglia (A) and astrocytes (B) were pretreated with inhibitor for 1.5 hrs and then Bexarotene for 24 hours followed by another 1.5 hours of pretreatment with inhibitors and 18 hours with 2 ug/mL soluble Aβ and Bexarotene.

FIG. 15 illustrates photographs showing Cryostat sections of brain (10 um) stained for GFAP. Animals were orally gavaged for 7 days with 100 mg/kg/day of Bexarotene (B) or vehicle (A). Hemi brain homogenates were subjected to Western analysis for GFAP. Actin served as a load control (not shown). The average optical density of GFAP in 4 animals per treatment group is significantly lower in the Bexarotene treated mice than in the vehicle (water) treated mice.

FIG. 16 illustrates photographs showing microglia in the brains of Bexarotene treated mice are able to phagocytose Aβ. Cryostat sections (10 um) were stained with 6E10 (plaque pathology) and Iba1, a marker for microglia. Using Z-stack, Aβ, marked by 6E10, is found within an Iba1-positive microglia (A). (100×).

### DETAILED DESCRIPTION

As used herein “agent” or “drug” is used herein to denote a chemical compound, a mixture of chemical compounds, a biological macromolecule, or an extract made from biological materials, such as bacteria, plants, fungi, or animal particularly mammalian cells or tissues that are suspected of having therapeutic properties. The agent or drug may be purified, substantially purified or partially purified.

As used herein, the term “purified” or “to purify” refers to the removal of one or more contaminants from a sample. The present invention contemplates purified compositions.

As used herein, the term “partially purified” refers to the removal of a moderate portion of the contaminants of a sample to the extent that the substance of interest is recognizable by techniques known to those skilled in the art as accounting for a measurable amount of the mixture. Preferably, the compound of interest is at least 5% of the total preparation and up to 50% of the total preparation. As used herein, the term “substantially purified” refers to the removal of a significant portion of the contaminants of a sample to the extent that the substance of interest is recognizable by techniques known to those skilled in the art as the most abundant substance in the mixture.

As used herein “agonist” refers to a molecule which, when interacting with a biologically active molecule, causes a change (e.g., enhancement) in the biologically active molecule, which modulates the activity of the biologically active molecule. Agonists include, but are not limited to proteins, nucleic acids, carbohydrates, lipids or any other molecules which bind or interact with biologically active molecules. For example, agonists can alter the activity of gene transcription by interacting with RNA polymerase directly or through a transcription factor or signal transduction pathway. Agonists can mimic the action of a “native” or “natural” compound. Agonists may be homologous to these natural compounds in respect to conformation, charge or other characteristics. Thus, agonists may be recognized by, e.g., nuclear receptors. This recognition may result in physiologic and/or biochemical changes within the cell, such that the cell reacts to the presence of the agonist in the same manner as if the natural compound was present.

The term “RXR agonist” refers to a compound or composition which, when combined with a Retinoid X Receptor (RXR), increases the transcriptional regulation activity of RXR homodimers and heterodimers.

As used herein, the term “therapeutically effective amount” refers to that amount of a composition that results in amelioration of symptoms or a prolongation of survival in a patient. A therapeutically relevant effect relieves to some extent one or more symptoms of a disease or condition or returns to normal either partially or completely one or more physiological or biochemical parameters associated with or causative of the disease or condition.

As used herein, the term “PPARγ agonist” refers to a compound or composition, which when combined with PPARγ, directly or indirectly stimulates or increases an in vivo or in vitro reaction typical for the receptor (e.g., transcriptional regulation activity). The increased reaction can be measured by any of a variety of assays known to those skilled in the art. An example of a PPARγ agonist is a thiazolidinedione compound, such as troglitazone, rosiglitazone, pioglitazone, ciglitazone, WAY-120,744, englitazone, AD 5075, darglitazone, and congeners, analogs, derivatives, and pharmaceutically acceptable salts thereof.

As used herein, the term “subject” refers to any animal, including, but not limited to, humans and non-human animals (e.g., rodents, arthropods, insects, fish (e.g., zebrafish), non-human primates, ovines, bovines, ruminants, lagomorphs, porcines, caprines, equines, canines, felines, ayes, etc.), which is to be the recipient of a particular treatment. Typically, the terms “patient” and “subject” are used interchangeably herein in reference to a human subject.

“ABCA1” is used herein to mean “ATP-binding cassette transporter A1”, and is also referred to in the art as “ABC1”.

“Activate”, when used in connection with a receptor, means to change the receptor's conformation so as to promote transcriptional activity.

“LXR” is used herein to mean “liver X receptors.”

As used herein, the term “in vitro” refers to an artificial environment and to processes or reactions that occur within an artificial environment. In vitro environments consist of, but are not limited to, test tubes and cell culture. The term “in vivo” refers to the natural environment (e.g., an animal or a cell) and to processes or reaction that occur within a natural environment.

“Treating” or “treatment” of a condition or disease includes: (1) preventing at least one symptom of the conditions, i.e., causing a clinical symptom to not significantly develop in a subject that may be exposed to or predisposed to the disease but does not yet experience or display symptoms of the disease, (2) inhibiting the disease, i.e., arresting or reducing the development of the disease or its symptoms, or (3) relieving the disease, i.e., causing regression of the disease or its clinical symptoms. Treatment, prevention and ameliorating a condition, as used herein, can include, for example decreasing or eradicating a deleterious or harmful condition associated with a PPARγ/RXR related disease(s) or disorder(s).

For the purposes of this application, the terms “PPARγ/RXR related disease(s) or disorder(s)” includes diseases and/or conditions related to the transcription of LXR target genes (e.g., ApoE, ABCA1, and ABCG1).

As used herein, the term “dermatological disorder” refers to any disorder of skin, hair, or glands. A dermatological disorder can be manifest in the form of visible lesions, pre-emergent lesions, pain, sensitivity to touch, irritation, inflammation, or the like. Dermatological disorders include disorders of the cutaneous and pilosebaceous unit or the process of keratogenesis. For example, a dermatological disorder can be a disorder of the epidermis or dermis, or within and surrounding a pilosebaceous unit, which is located within the epidermis, dermis, subcutaneous layer, or a combination thereof. Examples of dermatological disorders include, but are not limited to, acne, alopecia, psoriasis, seborrhea, ingrown hairs and pseudofolliculitis barbae, hyperpigmented skin, cutaneous infections, lichen planus, Graham Little Syndrome, periorificial dermatitis, rosacea, hidradenitis suppurativa, dissecting cellulitis, systemic lupus erythematosus, discoid lupus erythematosus, and the like.

As used herein, the term “alopecia” refers to partial or full baldness, hair loss, and/or hair thinning.

As used herein, the term “primary cicatricial alopecia” refers to a group of hair disorders that cause permanent destruction of the hair follicle. The term includes hair disorders in which the hair follicles are the primary target of a destructive inflammatory process. Cicatricial alopecias (CA) can be classified as lymphocytic, neutrophilic, and combinations thereof (i.e., “mixed”). Examples of lymphocytic CAs include lichen planopilaris, frontal fibrosing alopecia, chronic cutaneous lupus, erythematosus, pseudopelade, central centrifugal alopecia, alopecia mucinosa, and keratosis follicularis spinulosadecalvans. Examples of neutrophilic CAs include folliculitis decalvans, tufted folliculitis, and dissecting cellulitis. Examples of mixed CAs include follicullitis keloidalis and erosive dermatosis.

The present invention relates to compositions and methods of treating PPARγ and/or RXR related diseases and disorders. PPARγ and/or RXR related diseases and disorders can include, but are not limited to, neurodegenerative diseases and disorders, diseases and disorder resulting from trauma and injury, and/or an inflammatory component as well as dermatological diseases and disorders with or without an inflammatory component.

In one aspect of the invention, the compositions and methods can be used to regulate the production of proinflammatory and neurotoxic products involved in neurodegenerative diseases and disorders in a subject. Neurodegenerative disorders include, but are not limited to, Alzheimer's, Parkinson's, Huntington's Disease, as well as neural diseases and conditions with an inflammatory components, including, but not limited to, central nervous system injuries, stroke, ischemic damage to the nervous system, neural trauma (e.g., percussive brain damage, spinal cord injury, and traumatic damage to the nervous system), multiple sclerosis and other immune-mediated neuropathies (e.g., Guillain-Barre syndrome and its variants, acute motor axonal neuropathy, acute inflammatory demyelinating polyneuropathy, and Fisher Syndrome), HIV/AIDs dementia complex, and bacterial, parasitic, fungal, and viral meningitis and encephalitis.

In other aspects, the compositions and methods described herein can be administered to a subject to treat cystic fibrosis (CF) and CF-related disease(s) and disorder(s) (e.g., variant cystic fibrosis and non-CF bronchiectasis inflammatory responses), and inflammatory responses associate with associated with cystic fibrosis-related disease(s) or disorder(s). In still further aspects, the composition and methods described herein can be used to treat dermatological diseases and/or disorders where lipid PPARγ-regulated gene expression is decreased (e.g., LPP).

The compositions and methods of the present invention can include the use of RXR agonist alone or in combination with a PPARγ agonist (and optionally an LXR agonist) to suppress, inhibit, or mitigate a diverse range of PPARγ and/or RXR related diseases as described above and/or inflammatory responses associated with the PPARγ and/or RXR related diseases.

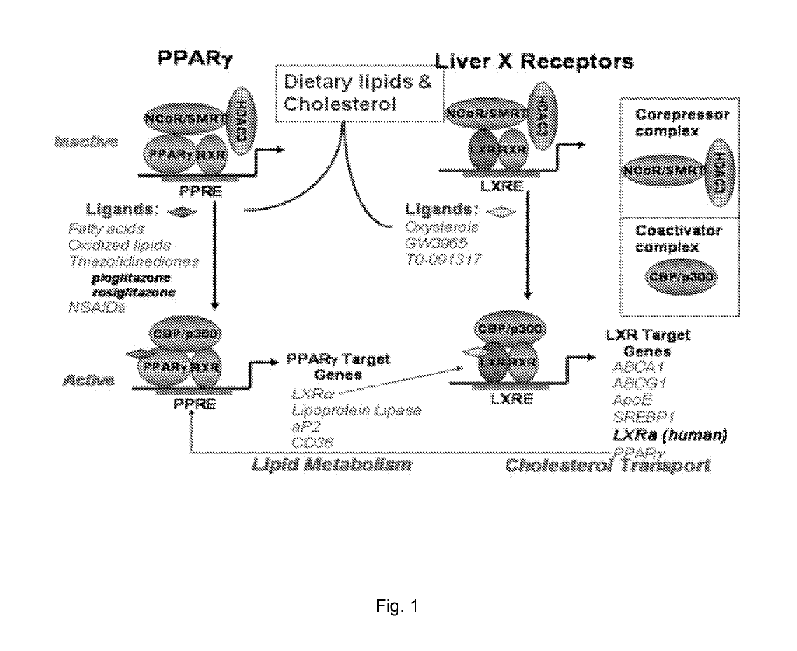

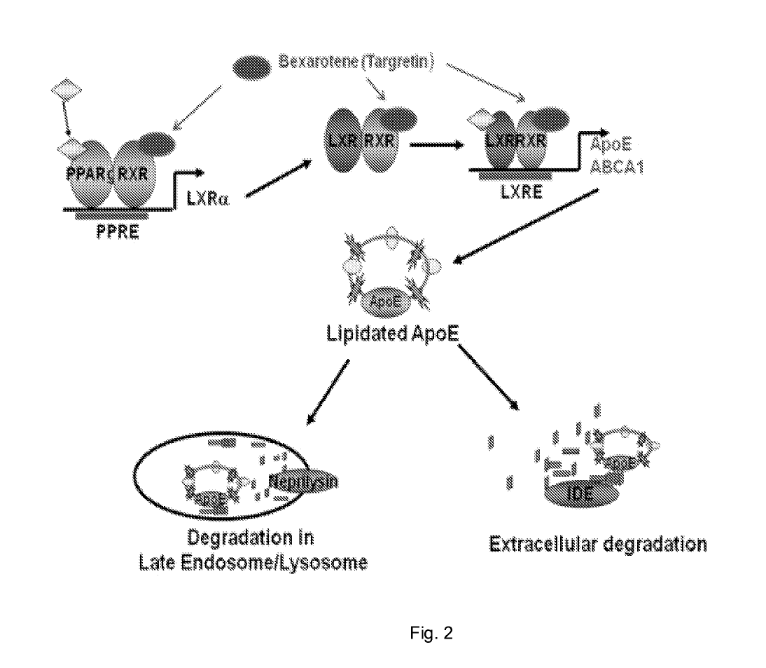

It was found that RXR nuclear receptors act in concert with other nuclear receptors (PPARγ and LXR) to facilitate the primary actions of the PPARγ and LXR receptors in a cell. PPARγ and LXRs are type II nuclear receptors, which form obligate heterodimers with RXR and form a functionally active transcription factor that is then competent to bind DNA and stimulate gene expression. It has been previously shown that PPARγ and LXRs act in concert to regulate lipid metabolism and ApoE expression (FIG. 1). It was also found that administration of RXR agonists, such as Bexarotene, to a subject can drive expression of LXR target genes (ABCA1, ABCG1, ApoE) and PPARγ target genes, which can promote the proteolytic degradation of beta amyloid (Aβ) in neuronal cells. Moreover, it was found that RXR agonists, such as Bexarotene, act additively or synergistically to enhance the actions of LXR agonists or PPARγ agonists in treating Alzheimer's and neurodegenative disorders or injuries. For example, ligation of both LXR and RXR results in a synergistic increase in the expression of ApoE and Aβ clearance from cells as well as ameliorates the behavioral impairments in in vivo models of Alzheimer's disease.

An aspect of the invention relates to a method of treating PPARγ and/or RXR related diseases and disorders by administering to a subject with the disorder a therapeutically effective amount of RXR agonist. Administration of RXR agonists can increase LXR target gene expression in the subject, improve the therapeutic efficacy of PPARγ agonist and LXR agonist agents in the treatment of PPARγ/RXR related diseases and disorders. Advantageously, the RXR agonist can be administered in combination with a PPARγ agonists and optionally an LXR agonist to synergistically treat the PPARγ and/or RXR related diseases and disorders. It is contemplated by the present invention that the administration of RXR agonists, by increasing LXR target gene expression in the subject, can improve the therapeutic efficacy of PPARγ agonist and LXR agonist agents in the treatment of PPARγ/RXR related diseases and disorders. The present invention therefore relates to therapies that utilize the synergistic properties of two or more therapeutic agents for the treatment of PPARγ/RXR related diseases and disorders.

The RXR agonist can include known RXR agonists that are described in, for example, the following U.S. patents and patent applications, which are incorporated by reference herein: U.S. Pat. Nos. 5,399,586, 5,466,861, 5,780,676, and 5,801,253; U.S. patent application Ser. Nos. 07/809,980, 08/003,223, 08/027,747, 08/045,807, 08/052,050, 08/052,051, 08/179,750, 08/366,613, 08/480,127, 08/481,877, 08/872,707, and 08/944,783. See also, WO 93/11755, WO 93/21146, WO 94/15902, WO 94/23068, WO 95/04036, and WO 96/20913.

Other RXR agonists that can be used in the present invention can include RXR agonists described for example, in the following articles: Boehm et al. J. Med. Chem. 38:3146 (1994), Boehm et al. J. Med. Chem. 37:2930 (1994), Antras et al., J. Biol. Chem. 266:1157-61 (1991), Salazar-Olivo et al., Biochem. Biophys. Res. Commun. 204: 10 257-263 (1994), and Safanova, Mol. Cell. Endocrin. 104:201 (1994). Such compounds may be prepared according to methods known in the art as described in the aforementioned references, as well as in M. L. Dawson and W. H. Okamura, Chemistry and Biology of Synthetic Retinoids, Chapters 3, 8, 14 and 16, CRC Press, Inc., Florida (1990); M. L. Dawson and P. D. Hobbs, The Retinoids, Biology, Chemistry and Medicine, M. B. Sporn et al., Eds. (2nd ed.), Raven Press, New York, N.Y., pp. 5-178 (1994); Liu et al., Tetrahedron, 40:1931 (1984); Cancer Res., 43:5268 (1983); Eur. J. Med. Chem. 15:9 (1980); Allegretto et al., J. Bio. Chem., 270:23906 (1995); Bissonette et al., Mol. Cell. Bio., 15:5576 (1995); Beard et al., J. Med. Chem., 38:2820 (1995), Koch et al., J. Med. Chem., 39:3229 (1996); and U.S. Pat. Nos. 4,326,055 and 4,578,498.

In some aspects of the invention, the RXR agonists can include LGD1069 (also known as Bexarotene), LGD100268, and LGD100324. The structures of RXR agonists designated LGD1069, LGD100268, and LGD100324 are shown below, and the synthesis of these compounds is described in U.S. Pat. Nos. 7,655,699 and 5,780,676. The synthesis of compounds LGD1069, LGD100268, and LGD100324 is also described in, e.g., WO 94/15902 and Boehm et al., J. Med. Chem. 38(16):3146 (1994).

In some aspects of the invention, a RXR agonist can include compounds of the following general formulas:

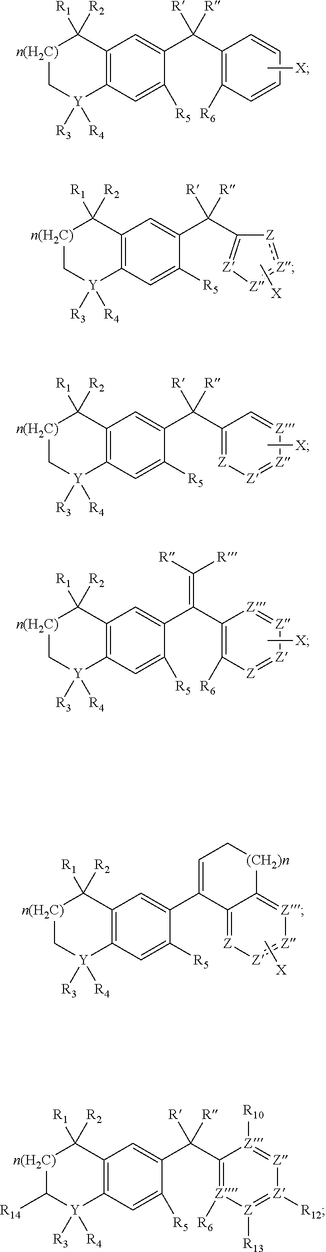

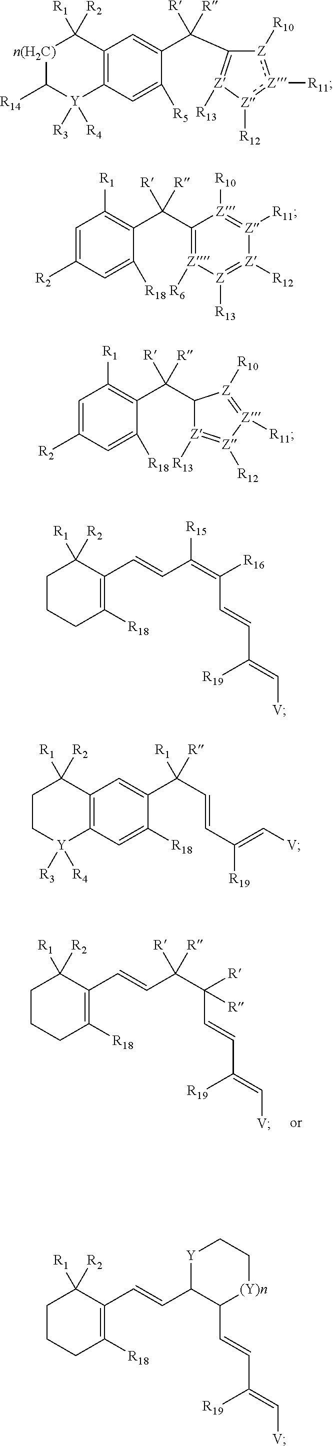

wherein R₁ and R₂, each independently, represent hydrogen or lower alkyl or acyl having 1-4 carbon atoms;

Y represents C, O, S, N, CHOH, CO, SO, SO₂, or a pharmaceutically acceptable salt;

R₃ represents hydrogen or lower alkyl having 1-4 carbon atoms where Y is C or N;

R₄ represents hydrogen or lower alkyl having 1-4 carbon atoms where Y is C, but R₄ does not exist if Y is N, and neither R₃ or R₄ exist if Y is S, O, CHOH, CO, SO, or SO₂;

R′ and R″ represent hydrogen, lower alkyl or acyl having 1-4 carbon atoms, OH, alkoxy having 1-4 carbon atoms, thiol or thio ether, or amino, or R′ or R″ taken together form an oxo (keto), methano, thioketo, HO—N═, NC—N═, (R₇R₈) N—N═, R₁₇O—N═, R₁₇N═, epoxy, cyclopropyl, or cycloalkyl group and wherein the epoxy, cyclopropyl, and cycloalkyl groups can be substituted with lower alkyl having 1-4 carbons or halogen;

R′″ and R″″ represent hydrogen, halogen, lower alkyl or acyl having 1-4 carbon atoms, alkyl amino, or R′″ and R″″ taken together form a cycloalkyl group having 3-10 carbons, and wherein the cycloalkyl group can be substituted with lower alkyl having 1-4 carbons or halogen;

R₅ represents hydrogen, a lower alkyl having 1-4 carbons, halogen, nitro, OR₇, SR₇, NR₇R₈, or (CF) nCF₃, but R₅ cannot be hydrogen if together R₆, R₁₀, R₁₁, R₁₂ and R₁₃ are all hydrogen, Z, Z′, Z″, Z′″, and Z″″ are all carbon, and R′ and R″ represent H, OH, C₁-C₄ alkoxy or C₁-C₄ acyloxy or R′ and R″ taken together form an oxo, methano, or hydroxyimino group;

R₆, R₁₀, R₁₁, R₁₂, R₁₃, each independently represent hydrogen, a lower alkyl having 1-4 carbons, halogen, nitro, OR₇, SR₇, NR₇R₈ or (CF)nCF₃, and exist only if the Z, Z′, Z″, Z′″, or Z″″ from which it originates is C, or each independently represent hydrogen or a lower alkyl having 1-4 carbons if the Z, Z′, Z″, Z′″, or Z″″ from which it originates is N, and where one of R₆, R₁₀, R₁₁, R₁₂ or R₁₃ is X;

R₇ represents hydrogen or a lower alkyl having 1-6 carbons;

R₈ represents hydrogen or a lower alkyl having 1-6 carbons;

R₉ represents a lower alkyl having 1-4 carbons, phenyl, aromatic alkyl, or q-hydroxyphenyl, q-bromophenyl, q-chlorophenyl, q-florophenyl, or q-iodophenyl, where q=2-4;

R₁₄ represents hydrogen, a lower alkyl having 1-4 carbons, oxo, hydroxy, acyl having 1-4 carbons, halogen, thiol, or thioketone;

R₁₅ represents a lower or branched alkyl having 1-12 carbons and can be methyl only if R16 is a halogen or a lower alkyl having 1-8 carbons;

R₁₆ represents hydrogen, a lower alkyl having 1-8 carbons, or halogen,

or R₁₅ and R₁₆ taken together form a phenyl, cyclohexyl, or cyclopental ring, or one of the following:

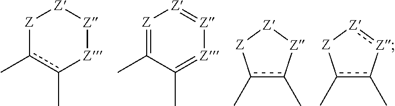

R₁₇ represents hydrogen, lower alkyl having 1-8 carbons, alkenyl (including halogen, acyl, OR₇ and SR₇ substituted alkenes), R₉, alkyl carboxylic acid (including halogen, acyl, OR₇ and SR₇ substituted alkyls), alkenyl carboxylic acid (including halogen, acyl, OR₇ and SR₇ substituted alkenes), alkyl amines (including halogen, acyl, OR₇ and SR₇ substituted alkyls), and alkenyl amines (including halogen, acryl, OR7 and SR7 substituted alkenes);

R₁₈ represents hydrogen, a lower alkyl having 1-4 carbons, halogen, nitro, OR₇, SR₇, NR₇R₈ or (CF)nCF3;

R₁₉ represents hydrogen, a lower alkyl having 1-8 carbons, halogen, OR₇, SR₇, or (CF)nCF₃;

X is COOH, tetrazole, PO₃H, SO₃H, CHO, CH₂OH, CONH₂, COSH, COOR₉, COSR₉, CONHR₉, or COOW where W is a pharmaceutically acceptable salt, and where X can originate from any C or N on the ring;

Z, Z′, Z″, Z′″ and Z″″, each independently, represent C, S, O, N, or a pharmaceutically acceptable salt, but is not O or S if attached by a double bond to another such Z or if attached to another such Z which is O or S, and is not N if attached by a single bond to another such Z which is N;

n=0-3; and the dashed lines depict optional double bonds.

In addition, thiophene, furanyl, pyridine, pyrazine, pyrazole, pyridazine, thadiazole, and pyrrole groups function as isosteres for phenyl groups, and may be substituted for the phenyl group of the above bicyclic benzyl derivatives.

Specific examples of RXR agonist compounds of the present invention are given in the following list:
p[3,5,5,8,8-pentamethyl-1,2,3,4-tetrahydro-2-naphthyl-(2-carbonyl)]-benzoic acid, also known as 4-[(3,5,5,8,8-pentamethyl-5,6,7,8-tetrahydro-2-naphthyl)carbonyl]benzoic acid, and designated “3-methyl-TTNCBN”;
p(5,5,8,8-tetramethyl-1,2,3,4-tetrahydro-3-isopropyl-2-naphthyl-(2-carbonyl)]-benzoic acid, also known as 4-[(3-isopropyl-5,5,8,8-tetramethyl-5,6,7,8-tetrahydro-2naphthyl)carbonyl]benzoic acid, and designated “3-XPRTTNCB”;
p[5,5,8,8-tetramethyl-1,2,3,4-tetrahydro-3-isopropyl 2-naphthyl-(2-methano)]-benzoic acid, also known as 4-[1(3-isopropyl-5,5,8,8-tetramethyl-5,6,7,8 tetrahydro-2-naphthyl)ethenyl]benzoic acid, and designated “3-IPRTTNEB”;
p[5,5,8,8-tetramethyl-1,2,3,4-tetrahydro-3-ethyl-2-naphthyl-(2-methano)]-benzoic acid, also known as 4-[1-(3-ethyl-5,5,8,8-tetramethyl-5,6,7,8-tetrahydro-2-naphthyl)ethenyl]benzoic acid, and designated “3-ethyl-TTNEB”;
p[(5,5,8,8-tetramethyl-1,2,3,4-tetrahydro-3-bromo-2-naphthyl-(2-methano)]-benzoic acid, also known as 4-[1-(3-bromo-5,5,8,8-tetramethyl-5,6,7,8-tetrahydro-2-naphthyl)ethenyl]benzoic acid, and designated “3-bromo-TTNEB”;
p[5,5,8,8-tetramethyl-1,2,3,4-tetrahydro-3]-chloro-2-naphthyl-(2-methano)-benzoic acid, also known as 4-[1(3-chloro-5,5,8,8-tetramethyl-5,6,7,8-tetrahydro-2-naphthyl]ethenyl benzoic acid, and designated “3-chloro-TTNEB”;
p[3,5,5,8,8-pentamethyl-1,2,3,4-tetrahydro-2-naphthyl-(2methano)]-benzoic acid, also known as 4-[1(3,5,5,8,8-pentamethyl-5,6,7,8-tetrahydro-2-naphthyl)ethenyl/benzoic acid, and designated “3-methyl-TTNEB”;
p[3,5,5,8,8-pentamethyl-1,2,3,4-tetrahydro-2-naphthyl-(2-hydroxymethyl)]-benzoic acid, also known as 4-[1-(3,5,5,8,8-pentamethyl-5,6,7,8-tetrahydro-2-naphthyl) hydroxymethyl]benzoic acid, and designated “3-methyl-TTNHMB”;
p[5,5,8,8-tetrarmethyl-1,2,3,4-tetrahydro-3-bromo-2-naphthyl-(2-carbonyl)]-benzoic acid, also known as 4-[(3-bromo-5,5,8,8-tetramethyl-5,6,7,8-tetrahydro-2-naphthyl) carbonyl]benzoic acid, and designated “3-bromo-TTNCB”;
p[5,5,8,8-tetramethyl-1,2,3,4-tetrahydro-3-chloro-2-naphthyl-(2-carbonyl)]-benzoic acid, also known as 4-[(3-chloro-5,5,8,8-tetramethyl-5,6,7,8-tetrahydro-2-naphthyl) carbonyl]benzoic acid, and designated “3-chloro-TTNCB”;
p[5,5,8,8-tetramethyl-1,2,3,4-tetrahydro-3-hydroxy-2-naphthyl-(2-carbonyl)]-benzoic acid, also known as 4-[(3-hydroxy-5,5,8,8-tetramethyl-5,6,7,8-tetrahydro-2-naphthyl)carbonyl]benzoic acid, and designated“3-hydroxy-TTNCB”;
p[5,5,8,8-tetramethyl-1,2,3,4-tetrahydro-3-ethyl-2-naphthyl-(2-carbonyl)]-benzoic acid, also known as 4-[(3-ethyl-5,5,8,8-tetramethyl-5,6,7,8-tetrahydro-2-naphthyl)carbonyl]benzoic acid, and designated “3-ethyl-TTNCB”;
p[3,5,5,8,8-pentamethyl-1,2,3,4-tetrahydro-2-naphthyl-(2-thioketo)]-benzoic acid, also known as 4-[(3,5,5,8,8-pentamethyl-5,6,7,8-tetrahydro-2-naphthyl)thioketo]benzoic acid, and designated “thioketone”;
p[3,5,5,8,8-pentamethyl-1,2,3,4-tetrahydro-2-naphthyl-(2-carbonyl)]-N-(4-hydroxyphenyl)benzamide, also known as 4-[(3,5,5,8,8-pentamethyl-5,6,7,8-tetrahydro-2-naphthyl)carbonyl]-N-(4-hydroxyphenyl)benzamide, and designated “3-methyl-TTNCHBP”;
p[3,5,5,8,8-pentamethyl-1,2,3,4-tetrahydro-2-naphthyl-(2-methano)]-N-(4-hydroxyphenyl)benzamide, also known as 4-[1-(3,5,5,8,8-pentamethyl-5,6,7,8-tetrahydro-2-naphthyl)ethenyl]-N-(4-hydroxyphenyl)benzamide, and designated “3-methyl-TTNEHBP”;
2-[1-(3,5,5,8,8-pentamethyl-5,6,7,8-tetrahydro-2-naphthyl)ethenyl]pyridine-5-carboxylic acid, designated “TPNEP”;
ethyl 2-[1-(3,5,5,8,8-pentamethyl-5,6,7,8-tetrahydro-2-naphthyl)ethenyl]pyridine-5-carboxylate, designated “TPNEPE”;
2-[1-(5,5,8,8-tetramethyl-5,6,7,8-tetrahydro-2-naphthyl)ethenyl]pyridine-5-carboxylic acid, designated “TTNEP”;
4-[1-(3,5,5,8,8-pentamethyl-5,6,7,8-tetrahydro-2-naphthyl)epoxy]benzoic acid, designated “TPNEB”;
4-[1-(3,5,5,8,8-pentamethyl-5,6,7,8-tetrahydro-2-naphthyl)cyclopropyl]benzoic acid, designated “TPNCB”;
4-[1-(3,5,5,8,8-pentamethyl-5,6,7,8-tetrahydro-2-naphthyl)ethenyl]benzenetetrazole, designated “3-methylTTNEBT”;
5-[1-(3,5,5,8,8-pentamethyl-5,6,7,8-tetrahydro-2-naphthyl)ethenyl]pyridine-2-carboxylic acid, designated “TPNEPC”;
2-[1-(3,5,5,8,8-pentamethyl-5,6,7,8-tetrahydro-2-naphthyl)cyclopropyl]pyridine-5-carboxylic acid, designated “TPNCP”;
methyl 2-[1-(3,5,5,8,8-pentamethyl-5,6,7,8-tetrahydro-2-naphthyl)cyclopropyl]pyridine-5-carboxylate;
3-methyl-7-propyl-9-(2,6,6-trimethyl-1-cyclohexen-1-yl)-2E,4E,6Z,8E-nonatetranoic acid;
3-methyl-7-isopropyl-9-(2,6,6-trimethyl-1-cyclohexen-yl)-2E,4E,6Z,8E-nonatetranoic acid;
3-methyl-7-t-butyl-9-(2,6,6-trimethyl-1-cyclohexen-1-yl)-2E,4E,6Z,8E-nonatetranoic acid;
3-methyl-5-{2-|2-(2,6,6-trimethylcyclohexen-1-yl)ethenyl|cyclohexyl}-2E,4E-pentadienoic acid;
(2E,4E)-3-methyl-5-[1-(3,5,5,8,8-pentamethyl-5,6,7,8-tetrahydro-2-naphthyl)cyclopropyl]penta-2,4-dienoic acid;
(2E,4E)-3-methyl-6-(1-[2,6,6-trimethyl-1-cyclohexenyl)ethenyl]cyclopropyl)-2,4-hexadienoic acid;
(2E,4E,6Z)-7-(5,5,8,8-tetramethyl-5,6,7,8-tetrahydro-2-naphthyl)-3,8-dimethyl-nona-2,4,6-trienoic acid;
(2E,4E,6Z)-7-(3,5,5,8,8-pentamethyl-5,6,7,8-tetrahydro-2-naphthyl)-3-methyl-octa-2,4,6-trienoic acid;
2-[1-(5,5,8,8-tetramethyl-5,6,7,8-tetrahydro-2-naphthyl)cyclopropyl]pyridine-5-carboxylic acid;
4-[(3,5,5,8,8-pentamethyl-5,6,7,8-tetrahydro-2-naphthyl)carbonyl]benzoic acid oxime;
4-[(3,5,5,8,8-pentamethyl-5,6,7,8-tetrahydro-2-naphthyl)carbonyl]benzoic acid methyloxime;
4-[1-(2-methyl-4-t-butylphenyl)ethenyl]benzoic acid;
4-[1-(2-methyl-4-t-butylphenyl)cyclopropyl]benzoic acid;
4-[(2-methyl-4-t-butylphenyl)carbonyl]benzoic acid;
4-[(2-methyl-4-t-butylphenyl)carbonyl]benzoic acid oxime; and
4-[1-(2-methyl-4-t-butylphenyl)carbonyl]benzoic acid methyloxime, designated Compound 144.

Representative structures for such compounds are as follows:

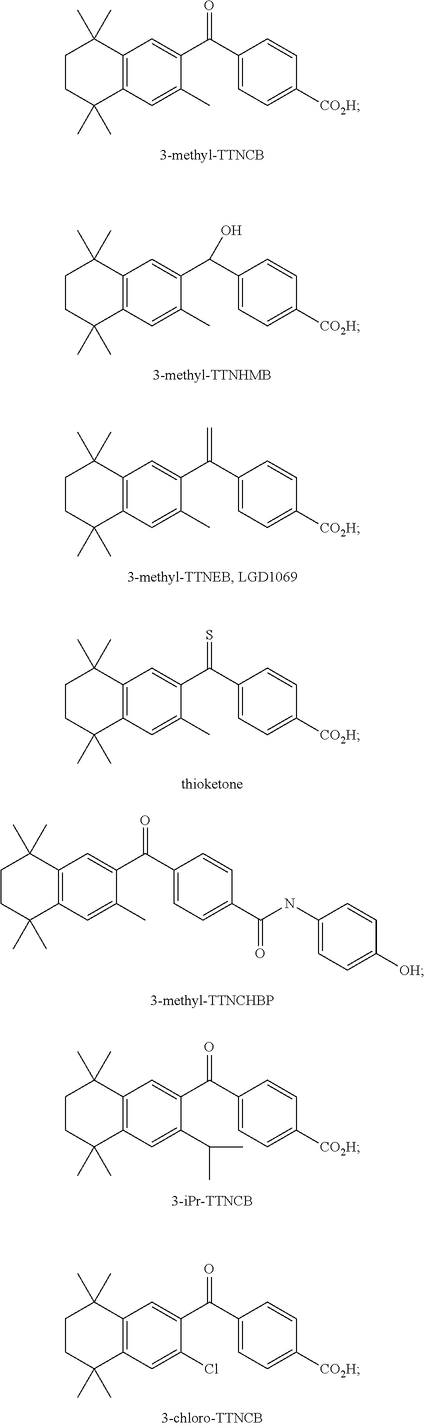

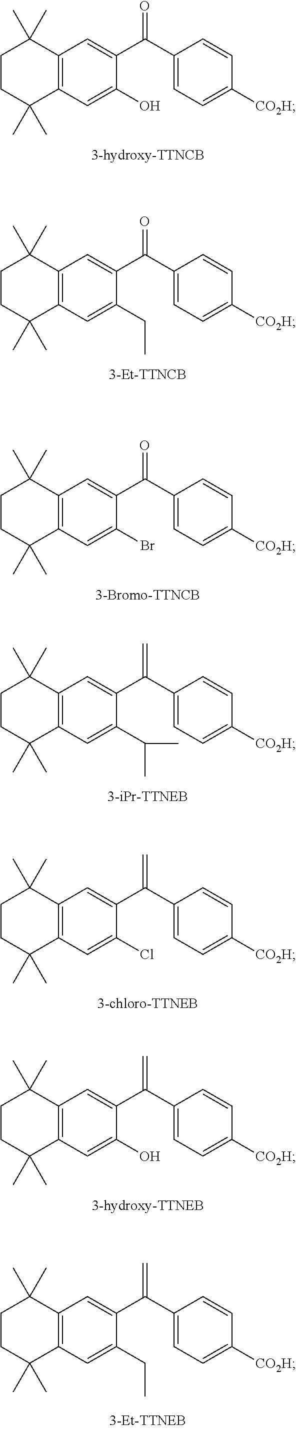

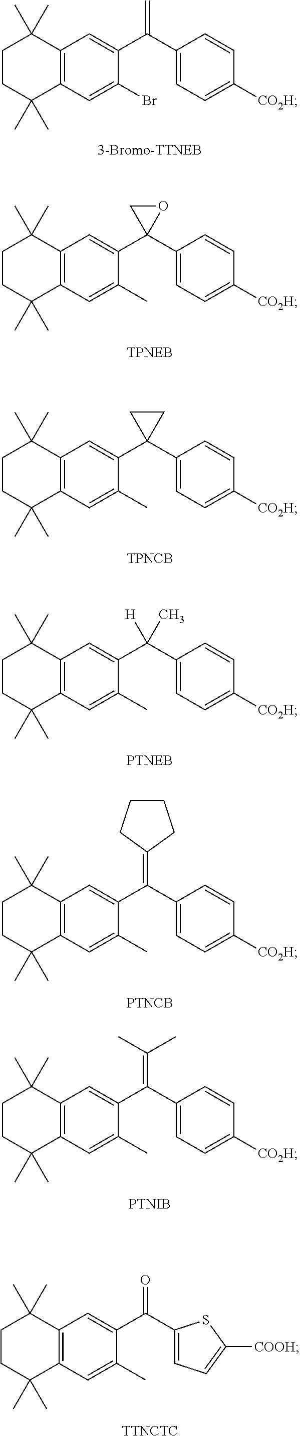

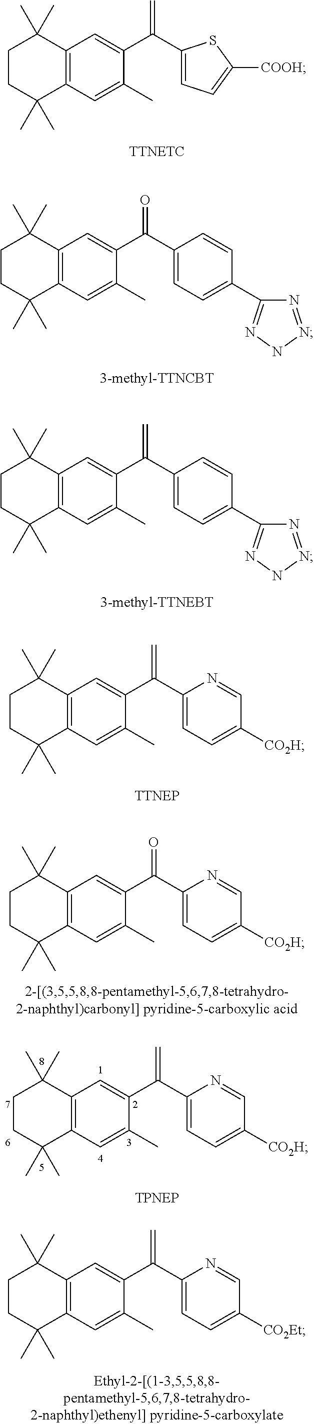

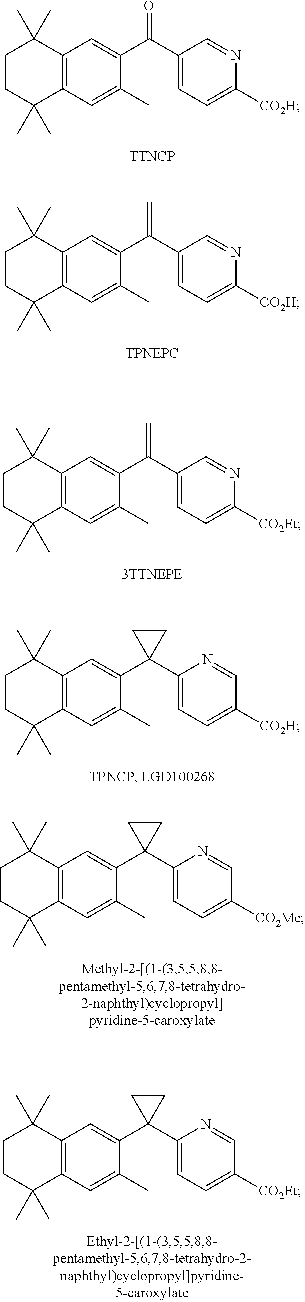

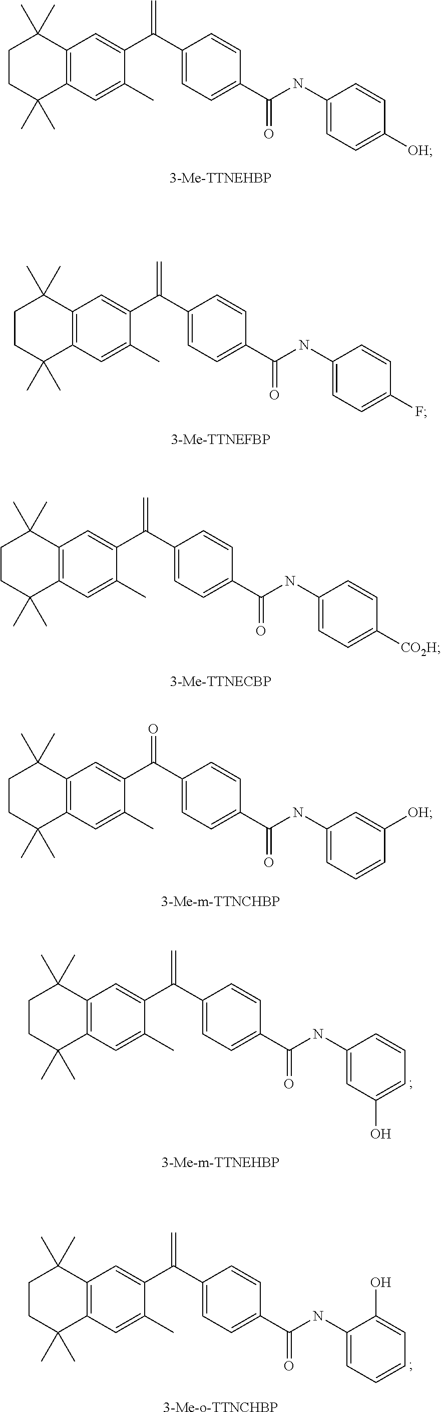

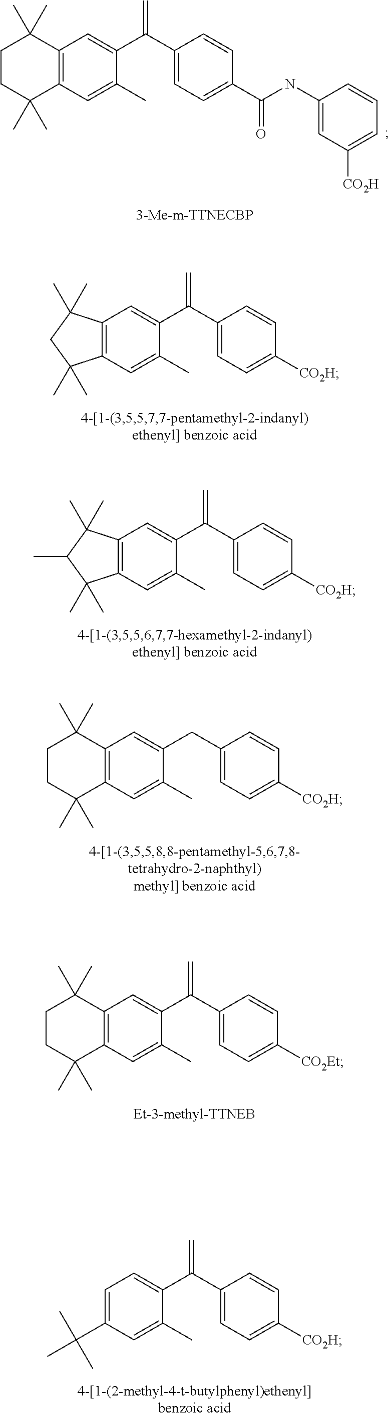

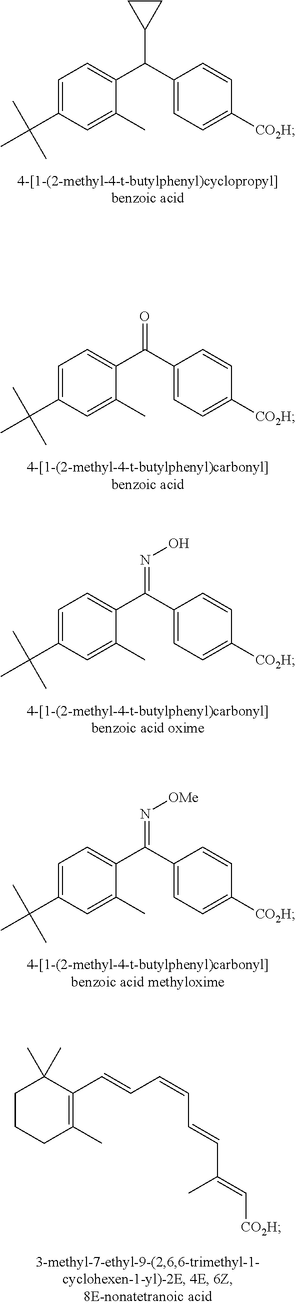

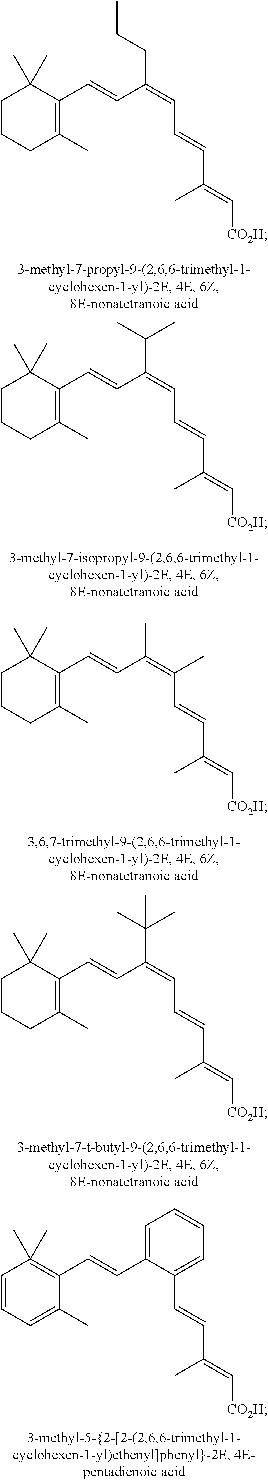

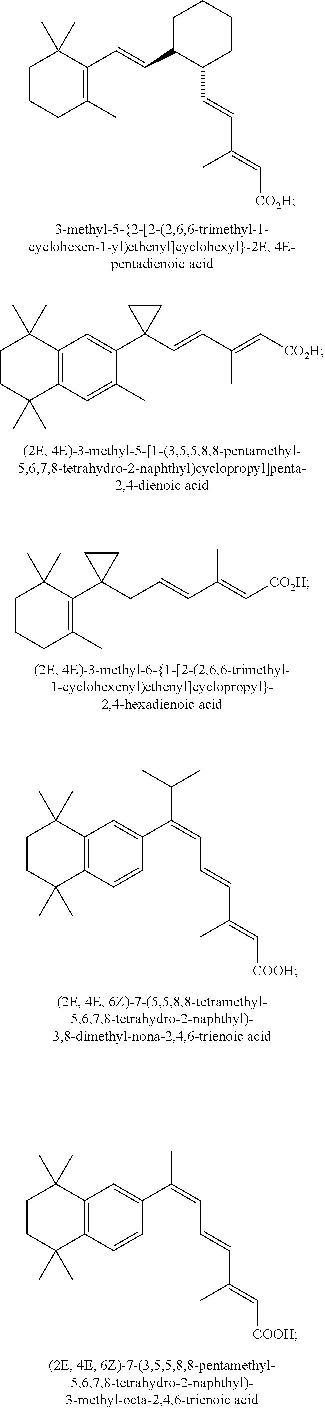

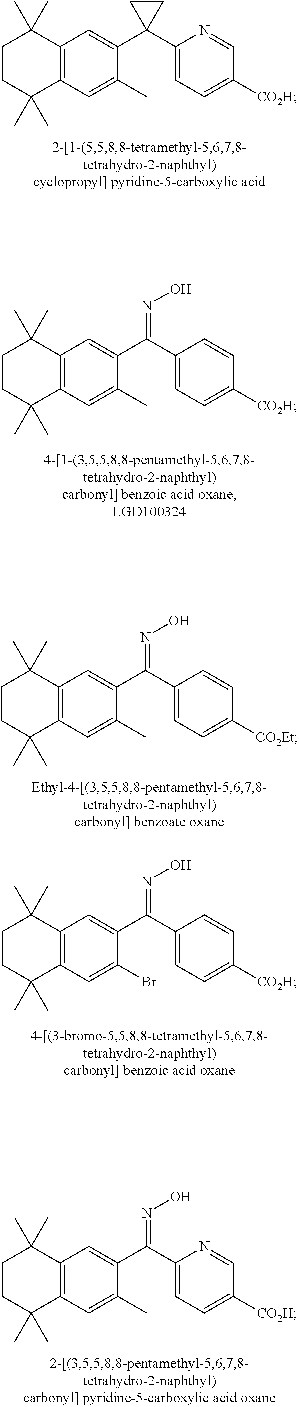

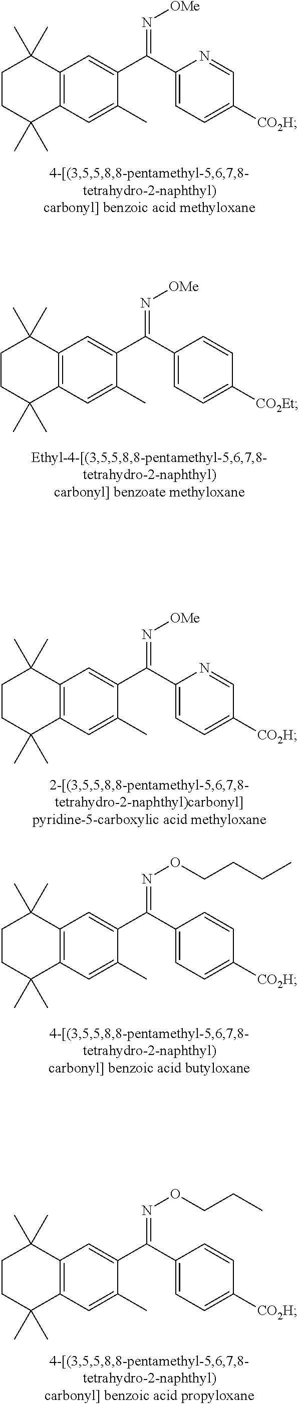

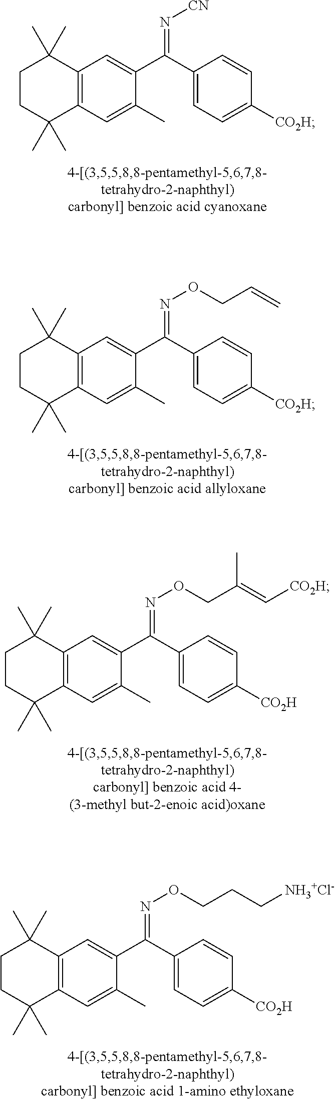

In addition, derivatives of the above compounds can be prepared according to U.S. Pat. Nos. 5,780,676; 5,962,731; 6,043,279; and 6,320,074 which are incorporated herein by reference.

In some aspects of the present invention, the RXR agonist can comprise compounds having the structure selected from the following formulas:

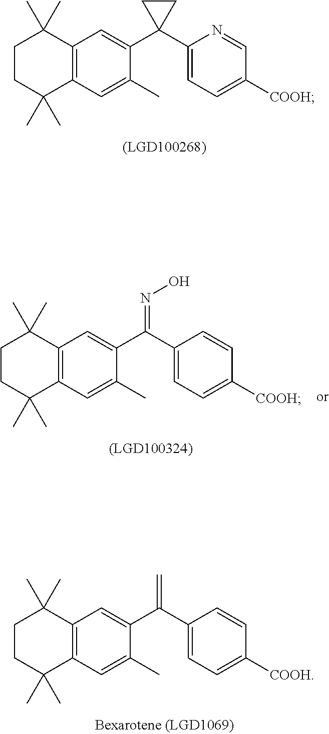

In certain aspects of the present invention, the RXR agonist can comprise a compound having the following structure;

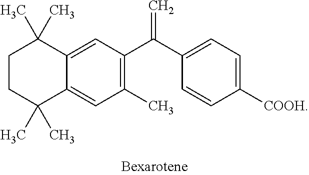

In another aspect of the present invention, the RXR agonist can include an agent disclosed in U.S. Pat. No. 7,348,359, having the following general formula (i):

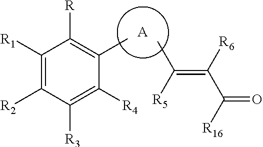

In formula (i), R is selected from the group of H, F, Cl, Br, I, C₁-C₃ alkyl, C₁-C₃ haloalkyl, C₂-C₃ alkenyl, C₂-C₃ haloalkenyl, C₂-C₃ alkynyl, C₂-C₃ haloalkynyl, and C₁-C₃ alkoxy, wherein said alkyl, haloalkyl, alkenyl, haloalkenyl, alkynyl, haloalkynyl, and alkoxy groups may be optionally substituted;

R₁ and R₂ are each, independently, H, a halo, a C₁-C₁₀ alkyl, a C₃-C₁₀ cycloalyl, a C₅-C₁₀ cycloalkenyl, a 6 to 10 membered aryl, a 5 to 10 membered heteroaryl, an aryl-C₁-C₆-alkyl, or an amino group represented by the formula NR₁₄R₁₅, wherein the alkyl, cycloalkyl, cycloalkenyl, aryl, heteroaryl and arylalkyl are optionally substituted with one or more halo, C₁-C₃ alky, C₁-C₃ haloalkyl or C₁-C₃ alkoxy; or R₁ and R₂ taken together with the carbon atoms to which they are attached form a five or six membered carbocyclic ring which is optionally substituted with one or more halo or C₁-C₆ alkyl groups. R₁₄ and R₁₅ are each, independently, H, a C₁-C₆ alkyl, or taken together with the nitrogen they are attached to form a 5 to 8 heterocycle.

Alternatively, R and R1 taken together with the carbon atoms to which they are attached form an aryl, a heteroaryl, a C₅-C₈ cycloalkyl or C₅-C₈ cycloalkenyl ring in which the aryl, heteroaryl, C₅-C₈ cycloalkyl or C₅-C₈ cyclolkenyl are optionally substituted with one or more halo, C₁-C₃ alkyl, C₁-C₃ haloalkyl or CI-C₃ alkoxy substituents. Preferably, when R and R₁ together with the carbon atoms to which they are attached form an aryl or a heteroaryl, the aryl and heteroaryl have from five to six atoms.

R₃ is H, a halo, a C₁-C₁₀ alkyl, a C₃-C₁₀ cycloalkyl, C₅-C₁₀ cycloalkenyl, a 6 to 10 membered aryl, a 5 to 10 membered heteroaryl, an aryl-C₁-C₆-alkyl, or an amino group represented by the formula NR₁₄R₁₅, wherein the alkyl, cycloalkyl, cycloalkenyl, aryl, heteroaryl and arylalkyl are optionally substituted with one or more halo, C₁-C₃ alkyl, C₁-C₃ haloalkyl or C₁-C₃ alkoxy.

R₄ is H, a halo, an aryl-C₁-C₆-alkyl, a C₁-C₁₀ alkyl or a C₁-C₁₀ alkoxy group wherein the arylalkyl, alkyl, and alkoxy are optionally substituted with one or more substituents selected from halo, C₁-C₆ alkyl, aryl, heteroaryl, a C₁-C₆ alkoxy, an amino group represented by the formula NR₁₄R₁₅. Preferably, the aryl and the heteroaryl substituents each, independently, have from five to ten atoms.

Alternatively, R₃ and R₄ taken together with the carbon atoms to which they are attached form an aryl, a heteroaryl, a C₅-C₈ cycloalkyl or C₅-C₈ cycloalkenyl ring wherein the aryl, heteroaryl, cycloalkyl and cycloalkenyl are optionally substituted with one or more halo, C₁-C₃ alkyl, C₁-C₃ haloalkyl or C₁-C₃ alkoxy substituents. Preferably, when R₃ and R₄ together with the carbon atoms to which they are attached form an aryl or a heteroaryl, the aryl and heteroaryl have from five to ten atoms.

R₅ is H, a halo, or a C₁-C₃ alkyl group, which is optionally substituted with one or more halo.

R₆ is H or halo.

R₁₆ is OR₁₇, OCH(R₁₇)OC(O)R₁₈, —NR₁₉R₂₀, or an aminoalkyl.

R₁₇, R₁₉ and R₂₀ are each, independently, H or a C₁-C₆ alkyl.

R₁₈ is a C₁-C₆ alkyl.

Ring A is a heteroaryl group represented by the following structural formula:

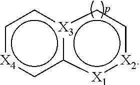

In ring A, X₁ and X₂ are each, independently, O, S, N, NH, or CH.

X₃ is N or C.

X₄ is CH or N.

P is 0 or 1.

However, when X₁ is O or S, then X₂ is CH or N and p is 0.

Ring A is optionally substituted with one or more substituents selected from a halo, a C₁-C₆ alkyl, or a C₁-C₆ alkoxy.

This group of compounds can be represented by the following formula (ii):

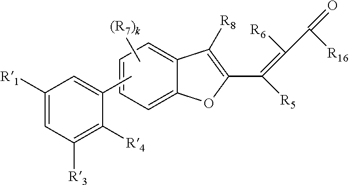

In this formula, R₅, R₆, and R₁₆, are as defined in formula (i).

R₁′ and R₃′ are each, independently, H, a halo, a C₁-C₁₀ alkyl, a C₃-C₁₀ cycloalkyl, a C₅-C₁₀ cycloalkenyl, a 6 to 10 membered aryl, a 5 to 10 membered heteroaryl, an aryl-C₁-C₆-alkyl or an amino group represented by the formula NR14R15 wherein the alkyl, cycloalkyl, cycloalkenyl, aryl, heteroaryl and arylalkyl are optionally substituted with one or more halo, C₁-C₃ alkyl, C₁-C₃ haloalkyl or C₁-C₃ alkoxy.

R₄′ is H, a halo, an aryl-C₁-C₆-alkyl, a C₁-C₁₀ alkyl or a C₁-C₁₀ alkoxy group wherein the arylalkyl, alkyl and alkoxy groups are optionally substituted with one or more substituents selected from halo, C₁-C₆ alkyl, aryl, heteroaryl, a C₁-C₆ alkoxy, an amino group represented by the formula NR₁₄R₁₅.

Each R₇ is, independently, a halo or a C₁-C₆ alkyl group.

R₈ is H, a halo or a C₁-C₆ alkyl group.

k is 0, 1, 2 or 3.

In a second preferred embodiment, compounds of the present invention and pharmaceutically acceptable salts, solvates and hydrates thereof, separately or with their respective pharmaceutical compositions, have a benzo[b]thienyl ring A. This group of compounds can be represented by formula (iii):

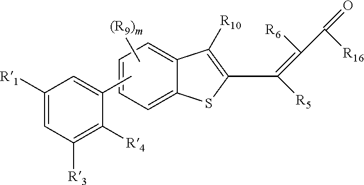

In formula (iii), R₅, R₆, and R₁₆, are as defined for Structural Formula i and R₁′, R₃′, and R₄′ are defined as in Structural Formula ii.

Each R₉ is, independently, a halo or a C1-C6 alkyl group;

R₁₀ is H, a halo or a C₁-C₆ alkyl group; and

m is 0, 1, 2 or 3.

In one aspect, compounds of the present invention and pharmaceutically acceptable salts, solvates and hydrates thereof, separately or with their respective pharmaceutical compositions, have an indolyl ring A. This group of compounds can be represented by formula (iv):

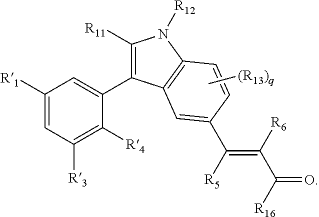

In formula (Iv), R₅, R₆, and R₁₆, are as defined for Structural Formula i and R₁′, R₃′, and R₄′ are defined as in Structural Formula ii.

R₁₁ is H, a halo or a C₁-C₆ alkyl.

R₁₂ is H or a C₁-C₆ alkyl.

Each R₁₃ is, independently, a halo or a C₁-C₆ alkyl group.

q is 0, 1, 2 or 3.

Specific examples of RXR agonist agents disclosed in U.S. Pat. No. 7,348,359 for use in the present invention are given in the following list:
3-[5-(2-hydroxy-3-tert-butyl-5-ethylphenyl)-benzo[b]furan-2-yl]-but-2-enoic acid;
2-fluoro-3-[5-(2-methoxy-3,5-di-iso-propylphenyl)benzo[b]furan-2-yl]-but-2-enoic acid;
2-fluoro-3-[7-(2-propoxy-3-tert-butyl-5-ethylphenyl)-benzo[b]furan-2-yl]-but-2-enoic acid ethyl ester;
3-[7-(2-ethoxy-3,5-di-tert-butylphenyl)-benzo[b]furan-2-yl]-but-2-enoic acid;
3-[7-(2-ethoxy-3,5-di-iso-propylphenyl)-benzo[b]furan-2-yl]-but-2-enoic acid;
3-[7-(2-propoxy-3,5-di-iso-propylphenyl)-benzo[b]furan-2-yl]-but-2-enoic acid;
3-{7-[2-(3-fluoropropoxy)-3,5-di-iso-propylphenyl]benzo[b]furan-2-yl}-but-2-enoic acid;
ethyl-2-carboxylate-7-(2-ethoxy-3,5-diisopropylbenzene)-benzo[b]thiophene;
3-{7-[2-(2,2-difluoroethoxy)-3,5-di-iso-propylphenyl]benzo[b]furan-2-yl}-but-2-enoic acid;
(E)-2-fluoro-3-{7-[2-(2,2-difluoroethoxy)-3,5-di-iso-propylphenyl]-benzo[b]furan-2-yl}-but-2-enoic acid;
(E)-3-{7-[5,5,8,8,-tetramethyl-3-ethoxy-5,6,7,8-tetrahydronaphth-2-yl]-benzo[b]furan-2-yl}-but-2-enoic acid;
3-[7-(2-ethoxy-3,5-di-iso-propylphenyl)-benzo[b]thien-2-yl]-but-2-enoic acid;
2-carboxy-4-(2-propoxy-3,5-di-tert-butylphenyl)-benzo[b]thiophene;
3-{4-[2-(2,2-difluoroethoxy)-3,5-di-tert-butylphenyl]benzo[b]thien-2-yl}-but-2-enoic acid;
(E)-3-[4-(2-propoxy-3,5-di-iso-propylphenyl)-benzo[b]thien-2-yl]-but-2-enoic acid;
(E)-3-[4-(2-ethoxy-3,5-di-iso-propylphenyl)-benzo[b]thien-2-yl]-but-2-enoic acid;
(E)-3-[4-(2-n-butoxy-3,5-di-iso-propylphenyl)-benzo[b]thien-2-yl]-but-2-enoic acid;
(E)-3-[4-(2-n-butoxy-3,5-di-iso-propylphenyl)-5-fluorobenzo[b]thien-2-yl]-but-2-enoic acid;
(E) 2-fluoro-3-[4-(2-n-propoxy-3,5-di-iso-propylphenyl)benzo[b]thien-2-yl]-prop-2-enoic acid;
(E) 3-[4-(2-propyloxy-3,5-di-iso-propylphenyl)benzo[b]thien-2-yl]prop-2-enoic acid;
3-{4-[2-(2,2,2-trifluoroethoxy)-3,5-di-iso-propylphenyl]benzo[b]thien-2-yl}-but-2-enoic acid;
3-{4-[2-(2,2,2-trifluoroethoxy)-3,5-di-iso-propylphenyl]benzo[b]furan-2-yl}-but-2-enoic acid;
3-{4-[2-(2,2,2-trifluoroethoxy)-3-tert-butyl-5-methylphenyl]benzo[b]thien-2-yl}but-2-enoic acid;
(E) 3-{4-[2-(2,2,2-trifluoroethoxy)-3,5-di-tert-butylphenyl]benzo[b]thien-2-yl}but-2-enoic acid;
(E) 3-{4-[2-(2,2,2-trifluoroethoxy)-3-tert-butyl-5-ethylphenyl]benzo[b]thien-2-yl}but-2-enoic acid;
(E) 3-{4-[2-(3-fluoropropoxy)-3-tert-butyl-5-ethylphenyl]benzo[b]thien-2-yl}but-2-enoic acid;
(E) 3-{4-[2-(2,2-difluoroethoxy)-3-(adamant-1-yl)-5-methylphenyl]benzo[b]thien-2-yl}but-2-enoic acid;
(E) 3-{4-[2-(3,3-difluoropropoxy)-3-tert-butyl-5-ethylphenyl]benzo[b]thien-2-yl}but-2-enoic acid;
(E) 3-{4-[2-(2,2-difluoroethoxy)-3-propyl-5-tert-butylphenyl]benzo[b]thien-2-yl}but-2-enoic acid;
(E) 3-{4-[2-(3,3-difluoropropoxy)-3-propyl-5-phenylphenyl]benzo[b]thien-2-yl}but-2-enoic acid;
(E) 3-{4-(2-(2,2,2-trifluoroethoxy)-3-phenyl-5-methylphenyl]benzo[b]thienyl}but-2-enoic acid;
(E) 3-{4-[2-(2-methylpropoxy)-3-tert-butyl-5-ethylphenyl]-benzo[b]thien-2-yl}but-2-enoic acid;
(E) 3-{4-[2-(2,2,2-trifluoroethoxy)-4-tert-butylphenyl]benzo[b]thien-2-yl}but-2-enoic acid;
(E) 3-[4-(5-(2,2,2-trifluoroethoxy)-6-tert-butylindan-4yl)-benzo[b]thien-2-yl]but-2-enoic acid;
(E) 3-[4-(3,5-di-tert-butylphenyl)-benzo[b]thien-2-yl]but-2-enoic acid;
(E) 3-{4-[3,5-di-iso-propyl-2-(2,2,2-trifluoroethoxy)phenyl]-5-fluoro-benzo[b]thien-2-yl}but-2-enoic acid;
(E) 3-{4-[2-(3-methylbutoxy)-3,5-di-tert-butylphenyl]benzo[b]thien-2-yl}but-2-enoic acid;
(E) 3-{4-[2-(3,3,3-difluoropropoxy)-3,5-di-tert-butylphenyl]benzo[b]thien-2-yl}but-2-enoic acid;
(E) 3-{4-[2-(2-methylpropoxy)-3,5-di-tert-butylphenyl)benzo[b]thien-2-yl]but-2-enoic acid;
(E) 3-{4-[2-(2,2,2-trifluoroethoxy)-3,5-di-(1,1-dimethylpropyl)phenyl]-benzo[b]thien-2-yl}but-2-enoic acid;
(E) 3-{4-[2-(2,2-difluoroethoxy)-3,5-di-(1,1-diethylpropyl)phenyl]-benzo[b]thien-2-yl}but-2-enoic acid;
(E) 3-{4-[2-(3-fluoropropoxy)-3,5-di-(1,1-dimethylpropyl)phenyl]-benzo[b]thien-2-yl}but-2-enoic acid;
(E) 3-{4-[2-(3-methylbutoxy)-3,5-di-(1,1-dimethylpropyl)phenyl]-benzo[b]thien-2-yl}but-2-enoic acid;
(E) 3-{4-[2-(3,3-difluoropropoxy)-3,5-di-(1,1-dimethylpropyl)-phenyl]-benzo[b]thiophene]but-2-enoic acid;
(E) 3-{4-[2-(2,2-difluoroethoxy)-3,5-di-(dimethylphenylmethyl)phenyl]-benzo[b]thien-2-yl}but-2-enoic acid;
(E) 3-{4-[2-(2,2-difluoroethoxy)-3-tert-butyl-5-phenylphenyl]-benzo[b]thien-2-yl}but-2-enoic acid;
(E) 3-{5-[2-(2,2-difluoroethoxy)-3-phenyl-5-tert-butylphenyl]-benzo[b]thien-2-yl}but-2-enoic acid;
3-[3-(2-butoxy-3,5-di-iso-propylphenyl)-1H-indol-5-yl]but-2-enoic acid;
3-[3-(2-butoxy-3,5-di-iso-propylphenyl)-1-methyl-1Hindol-5-yl]-but-2-enoic acid;
3-[3-(2-ethoxy-3,5-di-iso-propyl-phenyl)-1H-indol-5-yl]but-2-enoic acid;
3-[3-(2-butoxy-3,5-di-tert-butyl-phenyl)-1H-indol-5-yl]but-2-enoic acid;
3-[4-(2-butoxy-3,5-di-iso-propylphenyl)-1H-indol-2-yl]but-2-enoic acid;
3-[1-(2-butoxy-3,5-di-iso-propyl-phenyl)-isoquinolin-7yl]-but-2(E)-enoic acid;
3-[4-(2-butoxy-3,5-di-iso-propyl-phenyl)-quinolin-6-yl]but-2(E)-enoic acid;
3-{3-[2-(3-fluoropropoxy)-3,5-di-iso-propylphenyl]benzo[b]thien-5-yl}-but-2-enoic acid;
3-[3-(2-hydroxy-3,5-di-iso-propylphenyl)-benzo[b]thien5-yl]-but-2-enoic acid;
3-[3-(3,5-di-iso-propyl-2-methoxyphenyl)-benzo[b]thien-5-yl]-but-2-enoic acid;
3-[3-(2-ethoxy-3,5-di-iso-propyl-phenyl)-thieno[2,3-c]pyridin-5-yl]-but-2-enoic acid;
3-[3-(2-ethoxy-3,5-di-iso-propyl-phenyl)-benzo[d]isoxazol-5-yl]-but-2-enoic acid;
3-[3-(2-ethoxy-3,5-di-iso-propyl-phenyl)-1H-indazol-5yl]-but-2-enoic acid;
3-[3-(2-ethoxy-3,5-di-iso-propyl-phenyl)-imidazo[1,2-a]pyridin-6-yl]-but-2-enoic acid;
3-[3-(2-ethoxy-3,5-di-iso-propyl-phenyl)-imidazo[1,2-a]pyridin-6-yl]-acrylic acid;
3-[3-(3,5-di-tert-butyl-2-propoxy-phenyl)-1H-indol-5yl]-but-2-enoic acid;
3-{3-[3,5-di-tert-butyl-2-(2,2-difluoro-ethoxy)-phenyl]-1H-indol-5-yl}-but-2-enoic acid;
3-{3-[3,5-di-tert-butyl-2-(2,2,2-trifluoro-ethoxy)-phenyl]-1H-indol-5-yl}-but-2-enoic acid, and pharmaceutically acceptable salts, solvates and hydrates thereof.

In one aspect, ring A of the agents disclosed in U.S. Pat. No. 7,348,359 for use in the present invention is a benzo[b]furanyl. These compounds include, but are not limited to, the following compounds:
3-[5-(2-hydroxy-3-tert-butyl-5-ethylphenyl)-benzo[b]furan-2-yl]-but-2-enoic acid;
2-fluoro-3-[5-(2-methoxy-3,5-diisopropylphenyl)-benzo[b]furan-2-yl]-but-2-enoic acid;
2-fluoro-3-[7-(2-propoxy-3-tert-butyl-5-ethylphenyl)benzo[b]furan-2-yl]-but-2-enoic acid ethyl ester;
3-[7-(2-ethoxy-3,5-di-tert-butylphenyl)-benzo[b]furan-2yl]-but-2-enoic acid;
3-[7-(2-ethoxy-3,5-diisopropylphenyl)-benzo[b]furan-2yl]-but-2-enoic acid;
3-[7-(2-propoxy-3,5-diisopropylphenyl)-benzo[b]furan-2-yl]-but-2-enoic acid;
3-{7-[2-(3-fluoropropoxy)-3,5-diisopropylphenyl]-benzo[b]furan-2-yl}-but-2-enoic acid;
3-{7-[2-(2,2-difluoroethoxy)-3,5-diisopropylphenyl]benzo[b]furan-2-yl}-but-2-enoic acid;
(E)-2-fluoro-3-{7-[2-(2,2-difluoroethoxy)-3,5-diisopropylphenyl]-benzo[b]furan-2-yl}-but-2-enoic acid;
(E)-3-{7-[5,5,8,8,-tetramethyl-3-ethoxy-5,6,7,8-tetrahydronaphth-2-yl]-benzo[b]furan-2-yl}-but-2-enoic acid;
3-{4-[2-(2,2,2-trifluoroethoxy)-3,5-di-iso-propylphenyl]benzo[b]furan-2-yl}-but-2-enoic acid; and pharmaceutically acceptable salts, solvates and hydrates thereof.

In another embodiment, ring A of compounds of the present invention is a benzo[b]thienyl. These compounds include but are not limited to the following group of compounds:
ethyl-2-carboxylate-7-(2-ethoxy-3,5-di-iso-propylbenzene)-benzo[b]thiophene;
3-[7-(2-ethoxy-3,5-di-iso-propylphenyl)-benzo[b]thien-2-yl]-but-2-enoic acid;
2-carboxy-4-(2-propoxy-3,5-di-tert-butylphenyl)-benzo[b]thiophene;
(E)-3-[4-(2-propoxy-3,5-di-iso-propylphenyl)-benzo[b]thien-2-yl]-but-2-enoic acid;
(E)-3-[4-(2-ethoxy-3,5-di-iso-propylphenyl)-benzo[b]thien-2-yl]-but-2-enoic acid;
(E)-3-[4-(2-n-butoxy-3,5-di-iso-propylphenyl)-benzo[b]thien-2-yl]-but-2-enoic acid;
(E)-3-[4-(2-n-butoxy-3,5-di-iso-propylphenyl)-5-fluorobenzo[b]thien-2-yl]-but-2-enoic acid;
2-fluoro-3-[4-(3,5-di-iso-propyl-2-propyloxyphenyl)benzo[b]thien-2-yl]but-2-enoic acid
3-[4-(3,5-di-iso-propyl-2-propyloxyphenyl)-benzo[b]thien-2-yl]but-2-enoic acid;
3-{4-[2-(2,2,2-trifluoroethoxy)-3,5-di-iso-propylphenyl]benzo[b]thien-2-yl}-but-2-enoic acid;
(E) 2-{4-[2-(2,2,2-trifluoroethyloxy)-3-tert-butyl-5-methylphenyl]-benzo[b]thien-2-yl}but-2-enoic acid;
(E) 3-{4-[2-(2,2,2-trifluoroethyloxy)-3,5-di-tert-butylphenyl]-benzo[b]thien-2-yl}but-2-enoic acid;
(E) 3-{4-[2-(2,2,2-trifluoroethyloxy)-3-tert-butyl-5-ethylphenyl]-benzo[b]thien-2-yl}but-2-enoic acid;
(E) 3-{4-[2-(3-fluoropropyloxy)-3-tert-butyl-5-ethylphenyl]-benzo[b]thien-2-yl}but-2-enoic acid;
(E) 3-{4-[2-(2,2-difluoroethyloxy)-3-(adamant-1-yl)-5-methylphenyl]-benzo[b]thien-2-yl}but-2-enoic acid;
(E) 3-{4-[2-(3,3-difluoropropyloxy)-3-tert-butyl-5-ethylphenyl]-benzo[b]thien-2-yl}but-2-enoic acid;
(E) 3-{4-[2-(2,2-difluoroethyloxy)-3-propyl-5-tert-butylphenyl]-benzo[b]thien-2-yl}but-2-enoic acid;
(E) 3-{4-[2-(3,3-difluoropropyloxy)-3-propyl-5-phenylphenyl]-benzo[b]thien-2-yl}but-2-enoic acid;
(E) 3-{4-[2-(2,2,2-trifluoroethyloxy)-3-phenyl-5-methylbenzene]-benzo[b]thien-2-yl}but-2-enoic acid;
(E) 3-{4-[2-(2-methylpropyloxy)-3-tert-butyl-5-ethylphenyl]benzo[b]thien-2-yl}but-2-enoic acid;
(E) 3-{4-[2-(2,2,2-trifluoroethyloxy)-4-tert-butylphenyl]benzo[b]thien-2-yl}but-2-enoic acid; and pharmaceutically acceptable salts, solvates and hydrates thereof.

In another aspect, ring A of the agents disclosed in U.S. Pat. No. 7,348,359 for use in the present invention is an indolyl. These compounds include, but are not limited to, the following:
3-[3-(2-butoxy-3,5-di-iso-propyl-phenyl)-1H-indol-5-yl]but-2-enoic acid;
3-[3-(2-butoxy-3,5-di-iso-propylphenyl)-1-methyl-1H-indol-5-yl]but-2-enoic acid;
3-[3-(2-ethoxy-3,5-di-iso-propyl-phenyl)-1H-indol-5-yl]but-2-enoic acid;
3-[3-(2-butoxy-3,5-tert-butyl-phenyl)-1H-indol-5-yl]but-2-enoic acid;
3-[4-(2-butoxy-3,5-di-iso-propylphenyl)-1H-indol-2-yl]but-2-enoic acid;
3-[3-(3,5-di-tert-butyl-2-propoxy-phenyl)-1H-indol-5-yl]but-2-enoic acid;
3-{3-[3,5-di-tert-butyl-2-(2,2-difluoro-ethoxy)-phenyl]1H-indol-5-yl}-but-2-enoic acid;
3-{3-[3,5-di-tert-butyl-2-(2,2,2-trifluoro-ethoxy)-phenyl]-1H-indol-5-yl}-but-2-enoic acid; and pharmaceutically acceptable salts, solvates and hydrates thereof.

In some aspects, compounds represented by Structural Formula i have a ring A that is selected from the group consisting of an optionally substituted benzofuranyl, an optionally substituted benzo[b]thiophenyl, an optionally substituted indolyl, an optionally substituted thieno[2,3-c]pyridinyl, an optionally substituted benzold]isoxazolyl, an optionally substituted indazolyl, an optionally substituted imidazo[1,2-a]pyridinyl, an optionally substituted isoquinolinyl, or an optionally substituted quinolinyl.

In some aspects, compounds represented by formula (i) have a ring A that is selected from the following groups:

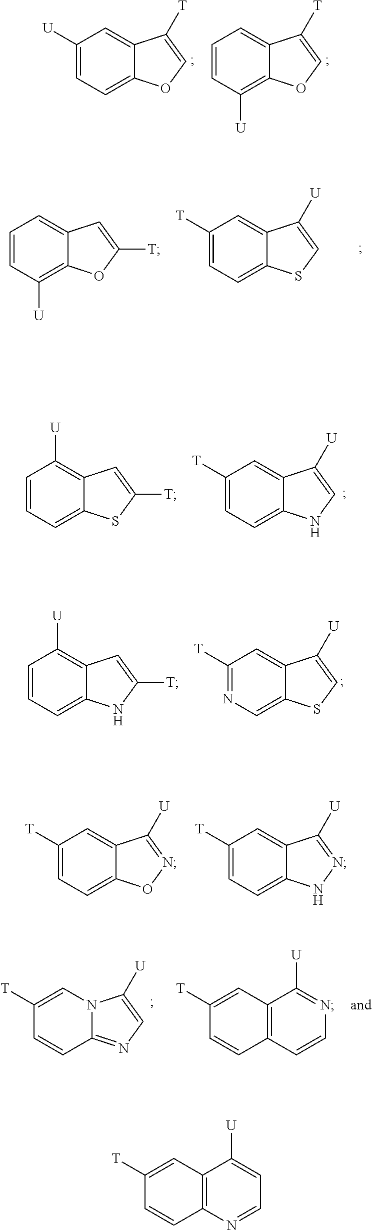

The symbol “U” indicates a single bond connecting ring A to the phenyl group, and the symbol “T” indicates a single bond connecting ring A to the a, αβ-unsaturated carbonyl group.

In another aspect, R₄ of formula (i) or R4 of preferred embodiments four and five is a C2-C5 alkoxy group, which is optionally substituted with one or more fluoro.

In another aspect, R₄′ of preferred embodiments one, two and three is a C₂-C₅ alkoxy group which is optionally substituted with one or more fluoro.

In another aspect, R₅ is methyl and R₆ is H in anyone of the previous embodiments.

In another aspect, R₅ is methyl and R₆ is fluoro in anyone of the previous embodiments.

In another aspect, R₁ and R₃ in anyone of the previous embodiments in which they occur are the same.

In another aspect, R₁ and R₃ in anyone of the previous embodiments in which they occur are the same and are iso-propyl or tert-butyl.

In another aspect, R₁′ and R₃′ in anyone of the previous embodiments in which they occur are the same.

In another embodiment, R₁′ and R₃′ in anyone of the previous embodiments in which they occur are the same and are iso-propyl or tert-butyl.

Optionally, a PPARγ agonist can be administered in combination with the RXR agonist to treat the PPARγ and/or RXR related diseases and disorders. PPARγ agonists for use in the present invention can include, for example, prostaglandin J2 (PGJ2) and analogs thereof (e.g., A2-prostaglandin J2 and 15-deoxy-2,4-prostaglandin J2), members of the prostaglandin D2 family of compounds, docosahexaenoic acid (DHA), and thiazolidinediones (e.g., ciglitazone, troglitazone, pioglitazone and rosiglitazone).

In addition, such PPARγ agonists can include, but are not limited to, L-tyrosine-based compounds, farglitazar, GW7845, indole-derived compounds, indole 5-carboxylic acid derivatives and 2,3-disubstituted indole 5-phenylacetic acid derivatives. It is appreciated that most of the PPARγ agonists exhibit substantial bioavailability following oral administration and have little or no toxicity associated with their use (See, e.g., Saltiel and Olefsky, Diabetes 45:1661 (1996); Wang et al., Br. J. Pharmacol. 122:1405 (1997); and Oakes et al., Metabolism 46:935 (1997)). It will be appreciated that the present invention is not limited to above-identified PPARγ agonists and that other identified PPARγ agonists can also be used.

PPARγ agonists that can be used for practicing the present invention, and methods of making these compounds, are disclosed in WO 91/07107; WO 92/02520; WO 94/01433; WO 89/08651; WO 96/33724; WO 97/31907; U.S. Pat. Nos. 4,287,200; 4,340,605; 4,438,141; 4,444,779; 4,461,902; 4,572,912; 4,687,777; 4,703,052; 4,725,610; 4,873,255; 4,897,393; 4,897,405; 4,918,091; 4,948,900; 5,002,953; 5,061,717; 5,120,754; 5,132,317; 5,194,443; 5,223,522; 5,232,925; 5,260,445; 5,814,647; 5,902,726; 5,994,554; 6,294,580; 6,306,854; 6,498,174; 6,506,781; 6,541,492; 6,552,055; 6,579,893; 6,586,455, 6,660,716, 6,673,823; 6,680,387; 6,768,008; 6,787,551; 6,849,741; 6,878,749; 6,958,355; 6,960,604; 7,022,722; and U.S. Applications 20030130306, 20030134885, 20030109579, 20030109560, 20030088103, 20030087902, 20030096846, 20030092697, 20030087935, 20030082631, 20030078288, 20030073862, 20030055265, 20030045553, 1 20020169192, 20020165282, 20020160997, 20020128260, 20020103188, 20020082292, 20030092736, 20030069275, 20020151569, and 20030064935.

The disclosures of these publications are incorporated herein by reference in their entireties, especially with respect to the PPARγ agonists disclosed therein, which may be employed in the methods described herein.

In some aspects of the present invention, the PPARγ agonists can comprise compounds of Formula I:

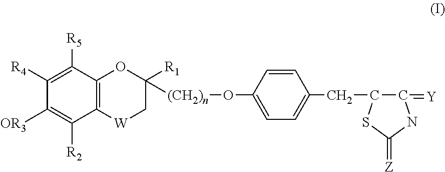

wherein R₁ and R₂ are the same or different, and each represents a hydrogen atom or a C₁-C₅ alkyl group; R₃ represents a hydrogen atom, a C₁-C₆ aliphatic acyl group, an alicyclic acyl group, an aromatic acyl group, a heterocyclic acyl group, an araliphatic acyl group, a (C₁-C₆ alkoxy)carbonyl group, or an aralkyloxycarbonyl group; R₄ and R₅ are the same or different, and each represents a hydrogen atom, a C₁-C₅ alkyl group or a C₁-C₅ alkoxy group, or R₄ and R₅ together represent a C₁-C₅ alkylenedioxy group; n is 1, 2, or 3; W represents the CH₂, CO, or CHOR₆ group (in which R₆ represents any one of the atoms or groups defined for R₃ and may be the same as or different, from R₃); and Y and Z are the same or different and each represents an oxygen atom or an imino (—NH) group; and pharmaceutically acceptable salts thereof.

In some aspects of the present invention, the PPARγ agonists can comprise compounds of Formula II:

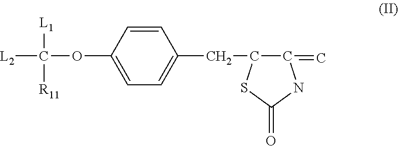

wherein R₁₁ is a substituted or unsubstituted alkyl, alkoxy, cycloalkyl, phenylalkyl, phenyl, aromatic acyl group, a 5- or 6 membered heterocyclic group including 1 or 2 heteroatoms selected from the group consisting of nitrogen, oxygen, and sulfur, or a group of the formula indicated in:

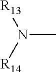

wherein R₁₃ and R₁₄ are the same or different and each is a lower alkyl (alternately, R₁₃ and R₁₄ are combined to each other either directly or as interrupted by a heteroatom comprising nitrogen, oxygen, and sulfur to form a 5- or 6-membered ring); and wherein L¹ and L² are the same or different and each is hydrogen or lower alkyl or L¹ and L² are combined to form an alkylene group; or a pharmaceutically acceptable salt thereof.

In some aspects of the present invention, the PPARγ agonists can comprise compounds of Formula III:

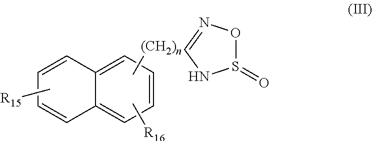

wherein R₁₅ and R₁₆ are independently hydrogen, lower alkyl containing 1 to 6 carbon atoms, alkoxy containing 1 to 6 carbon atoms, halogen, ethyl, nitrite, methylthio, trifluoromethyl, vinyl, nitro, or halogen substituted benzyloxy; n is 0 to 4; or a pharmaceutically acceptable salt thereof.

In some aspects of the present invention, the PPARγ agonists can comprise compounds of Formula IV:

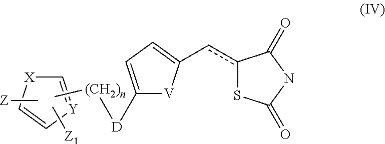

wherein the dotted line represents a bond or no bond; V is HCH—, —NCH—, —CH═N—, or S; D is CH₂, CHOH, CO, C═NOR₁₇, or CH═CH; X is S, SO, NR₁₈, —CH═N, or —N═CH; Y is CH or N; Z is hydrogen, (C₁-C₇)alkyl, (C₁-C₇)cycloalkyl, phenyl, naphthyl, pyridyl, furyl, thienyl, or phenyl mono- or di-substituted with the same or different groups which are (C₁-C₃)alkyl, trifluoromethyl, (C₁-C₃)alkoxy, fluoro, chloro, or bromo; Z₁ is hydrogen or (C₁-C₃)alkyl; R₁₇ and R₁₈ are each independently hydrogen or methyl; and n is 1, 2, or 3; the pharmaceutically acceptable cationic salts thereof; and the pharmaceutically acceptable acid addition salts thereof when the compound contains a basic nitrogen.

In some aspects of the present invention, the PPARγ agonists can comprise compounds of Formula V:

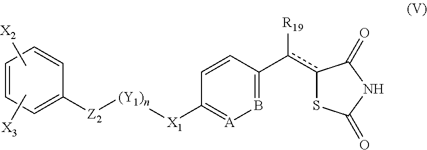

wherein the dotted line represents a bond or no bond; A and B are each independently CH or N. with the proviso that when A or B is N. the other is CH; X is S, SO, SO₂, CH₂, CHOH, or CO; n is 0 or 1; Y₁ is CHR₂₀ or R₂₁, with the proviso that when n is 1 and Y₁ is NR₂₁, X₁ is SO₂ or CO; Z₂ is CHR₂₂, CH₂CH₂, cyclic C₂H₂O, CH═CH, OCH₂, SCH₂, SOCH₂, or SO₂CH₂; R₁₉, R₂₀, R₂₁, and R₂₂ are each independently hydrogen or methyl; and X₂ and X₃ are each independently hydrogen, methyl, trifluoromethyl, phenyl, benzyl, hydroxy, methoxy, phenoxy, benzyloxy, bromo, chloro, or fluoro; a pharmaceutically acceptable cationic salt thereof; or a pharmaceutically acceptable acid addition salt thereof when A or B is N.

In other aspects of the present invention, the PPARγ agonists can comprise compounds of Formula VI:

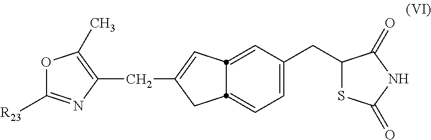

or a pharmaceutically acceptable salt thereof, wherein R₂₃ is alkyl of 1 to 6 carbon atoms, cycloalkyl of 3 to 7 carbon atoms, phenyl or mono- or all-substituted phenyl wherein said substituents are independently alkyl of 1 to 6 carbon atoms, alkoxy of 1 to 3 carbon atoms, halogen, or trifluoromethyl.

In some aspects of the present invention, the PPARγ agonists can comprise compounds of Formula VII:

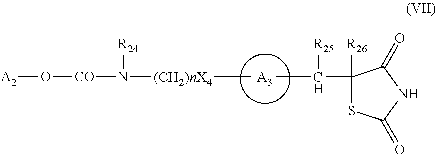

or a tautomeric form thereof and/or a pharmaceutically acceptable salt thereof, and/or a pharmaceutically acceptable solvate thereof, wherein: A₂ represents an alkyl group, a substituted or unsubstituted aryl group, or an aralkyl group wherein the alkylene or the aryl moiety may be substituted or unsubstituted; A³ represents a benzene ring having in total up to 3 optional substituents; R₂₄ represents a hydrogen atom, an alkyl group, an acyl group, an aralkyl group wherein the alkyl or the aryl moiety may be substituted or unsubstituted, or a substituted or unsubstituted aryl group; or A₂ together with R₂₄ represents substituted or unsubstituted C₂₋₃ polymethylene group, optional substituents for the polymethylene group being selected from alkyl or aryl or adjacent substituents together with the methylene carbon atoms to which they are attached form a substituted or unsubstituted phenylene group; R₂₅ and R₂₆ each represent hydrogen, or R₂₅ and R₂₆ together represent a bond; X₄ represents O or S; and n represents an integer in the range from 2 to 6.

In some aspects of the present invention, the PPARγ agonists can comprise compounds of Formula VIII:

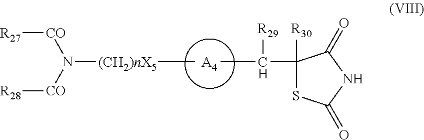

or a tautomeric form thereof and/or a pharmaceutically acceptable salt thereof, and/or a pharmaceutically acceptable solvate thereof, wherein: R₂₇ and R₂₈ each independently represent an alkyl group, a substituted or unsubstituted aryl group, or an aralkyl group being substituted or unsubstituted in the aryl or alkyl moiety; or R₂₇ together with R₂₈ represents a linking group, the linking group consisting or an optionally substituted methylene group or an O or S atom, optional substituents for the methylene groups including alkyl, aryl, or aralkyl, or substituents of adjacent methylene groups together with the carbon atoms to which they are attached form a substituted or unsubstituted phenylene group; R₂₉ and R₃₀ each represent hydrogen, or R₂₉ and R₃₀ together represent a bond; A₄ represents a benzene ring having in total up to 3 optional substituents; X₅ represents O or S; and n represents an integer in the range of 2 to 6.

In some aspects of the present invention, the PPARγ agonists can comprise compounds of Formula IX:

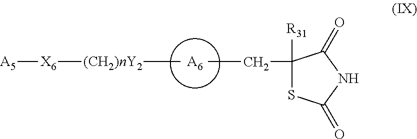

or a tautomeric form thereof and/or a pharmaceutically acceptable salt thereof, and/or a pharmaceutically acceptable solvate thereof, wherein: A₅ represents a substituted or unsubstituted aromatic heterocyclyl group; A₆ represents a benzene ring having in total up to substituents; X₆ represents O, S, or NR₃₂ wherein R₃₂ represents a hydrogen atom, an alkyl group, an acyl group, an aralkyl group, wherein the aryl moiety may be substituted or unsubstituted, or a substituted or unsubstituted aryl group; Y₂ represents O or S; R₃₁ represents an alkyl, aralkyl, or aryl group; and n represents an integer in the range from 2 to 6. Aromatic heterocyclyl groups include substituted or unsubstituted, single or fused ring aromatic heterocyclyl groups comprising up to 4 hetero atoms in each ring selected from oxygen, sulfur, or nitrogen. Aromatic heterocyclyl groups include substituted or unsubstituted single ring aromatic heterocyclyl groups having 4 to 7 ring atoms, preferably 5 or 6 ring atoms.

In particular, the aromatic heterocyclyl group comprises 1, 2, or 3 heteroatoms, especially 1 or 2, selected from oxygen, sulfur, or nitrogen. Values for A₅ when it represents a 5-membered aromatic heterocyclyl group include thiazolyl and oxazoyl, especially oxazoyl. Values for A₆ when it represents a 6-membered aromatic heterocyclyl group include pyridyl or pyrimidinyl. R₃₁ represents an alkyl group, in particular a C-6 alkyl group (e.g., a methyl group).

A5 can represent a moiety of formula (a), (b), or (c), under Formula IX:

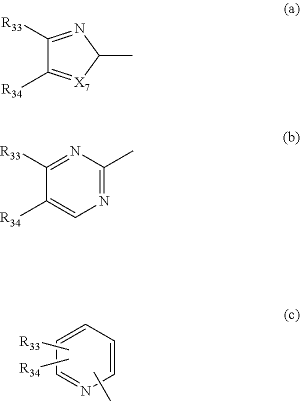

wherein, R₃₃ and R₃₄ each independently represents a hydrogen atom, an alkyl group, or a substituted or unsubstituted aryl group or when R₃₃ and R₃₄ are each attached to adjacent carbon atoms, then R₃₃ and R₃₄ together with the carbon atoms to which they are attached forth a benzene ring wherein each carbon atom represented by R₃₃ and R₃₄ together may be substituted or unsubstituted; and in the moiety of Formula (a), X₇ represents oxygen or sulfur.

In one aspect of the present invention, R₃₃ and R₃₄ together present a moiety of Formula (d), under Formula IX:

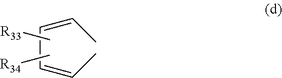

wherein R₃₅ and R₃₆ each independently represent hydrogen, halogen, substituted or unsubstituted alkyl, or alkoxy.

In some aspects of the present invention, the PPARγ agonists can comprise compounds of Formula X:

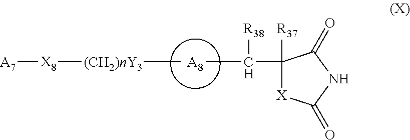

or a tautomeric form thereof and/or a pharmaceutically acceptable salt thereof, and/or a pharmaceutically acceptable solvate thereof, wherein: A₇ represents a substituted or unsubstituted aryl group; A₈ represents a benzene ring having in total up to 5 substituents; X₈ represents O, S, or NR₉, wherein R₃₉ represents a hydrogen atom, an alkyl group, an acyl group, an aralkyl group, wherein the aryl moiety may be substituted or unsubstituted, or a substituted or unsubstituted aryl group; Y₃ represents O or S; R₃₇ represents hydrogen; R₃₈ represents hydrogen or an alkyl, aralkyl, or aryl group or R₃₇ together with R₃₈ represents a bond; and n represents an integer in the range from 2 to 6.

In some aspects of the present invention, the PPARγ agonists can comprise compounds of Formula XI:

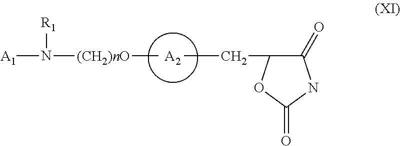

or a tautomeric form thereof and/or a pharmaceutically acceptable salt thereof, and/or a pharmaceutically acceptable solvate thereof, wherein: A₁ represents a substituted or unsubstituted aromatic heterocyclyl group; R₁ represents a hydrogen atom, an alkyl group, an acyl group, an aralkyl group, wherein the aryl moiety may be substituted or unsubstituted, or a substituted or unsubstituted aryl group; A₂ represents a benzene ring having in total up to 5 substituents; and n represents an integer in the range of from to 6. Suitable aromatic heterocyclyl groups include substituted or unsubstituted, single or fused ring aromatic heterocyclyl groups comprising up to 4 hetero atoms in each ring selected from oxygen, sulfur, or nitrogen. Favored aromatic heterocyclyl groups include substituted or unsubstituted single ring aromatic heterocyclyl groups having 4 to 7 ring atoms, preferably 5 or 6 ring atoms. In particular, the aromatic heterocyclyl group comprises 1, 2, or 3 heteroatoms, especially 1 or 2, selected from oxygen, sulfur, or nitrogen. Values for A₁ when it represents a 5-membered aromatic heterocyclyl group can include thiazolyl and oxazolyl, especially oxazoyl. Values for A₁ when it represents a 6-membered aromatic heterocyclyl group can include pyridyl or pyrimidinyl.

In some aspects of the present invention, the PPARγ agonists can comprise a compound of Formulas XII and XIII:

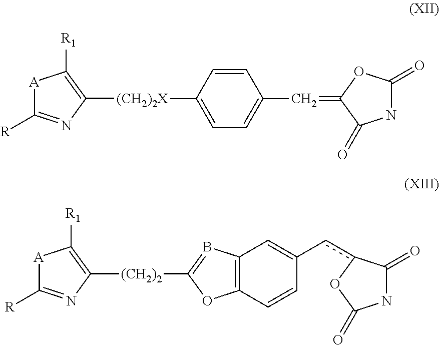

or pharmaceutically acceptable salts thereof wherein the dotted line represents a bond or no bond; R is cycloalkyl of three to seven carbon atoms, naphthyl, thienyl, furyl, phenyl, or substituted phenyl wherein the substituent is alkyl of one to three carbon atoms, alkoxy of one to three carbon atoms, trifluoromethyl, chloro, fluoro, or bis(trifluoromethyl); R₁ is an alkyl of one to three carbon atoms; X is O or C═O; A is O or S; and B is N or CH.

Some embodiments of the present invention include the use of the compounds of Formulas I through XIII are referred to as thiazolidine derivatives. Where appropriate, the specific names of thiazolidine derivatives may be used, including, for example, troglitazone, ciglitazone, pioglitazone, and rosiglitazone.

In certain aspects, an activator of a PPARγ agonist may be used as described in U.S. Pat. No. 5,994,554, e.g., having a structure selected from the group consisting of formulas (XIV)-(XXVI):

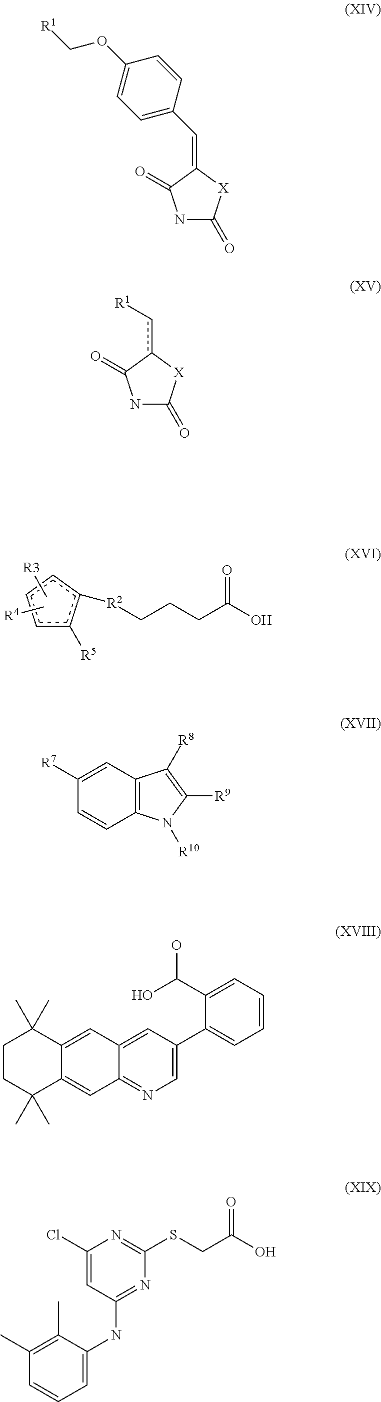

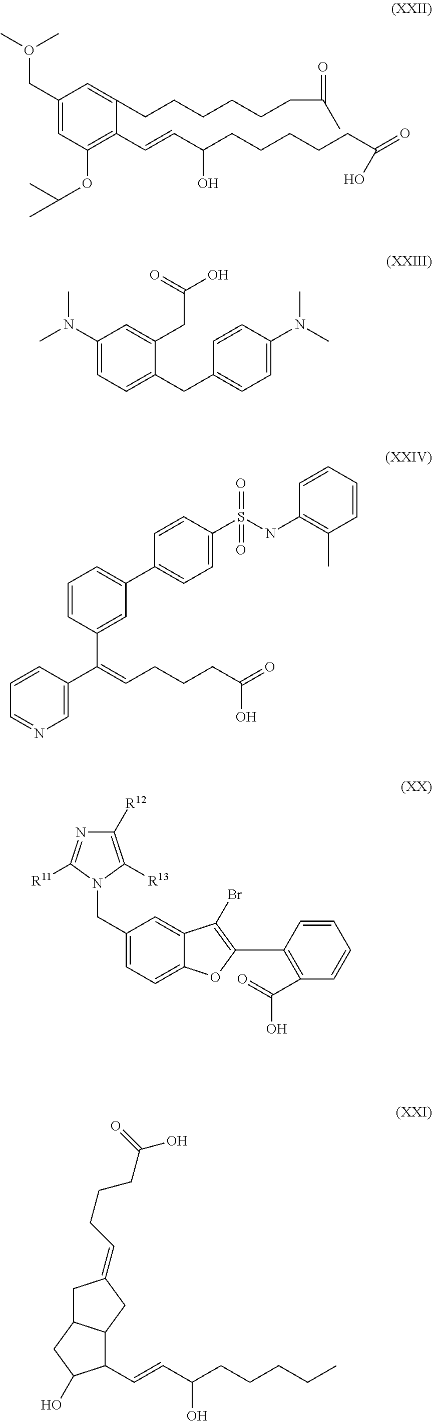

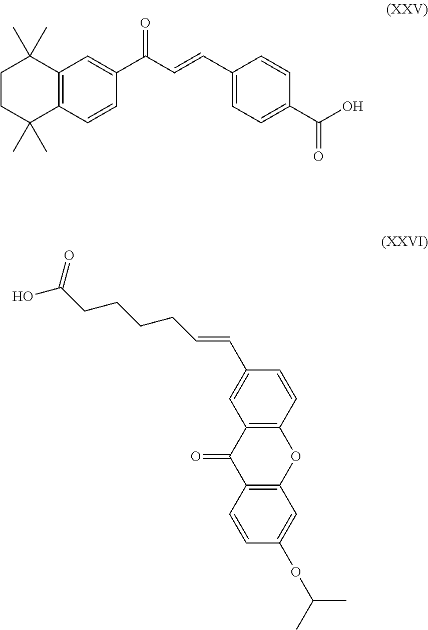

wherein: R¹ is selected from the group consisting of hydrogen, C₁₋₈ alkyl, aminoC₁₋₈, alkyl, C₁₋₈ alkylamino C₁₋₈ alkyl, heteroarylamino C₁₋₆ alkyl, (heteroaryl)(C₁₋₈alkyl)aminoC₁₋₆ alkyl, (C₁₋₈ cycloalkyl) C₁₋₈ alkyl, C₁₋₈ alkylheteroaryl C₁₋₈ alkyl, 9- or 10-membered heterobicycle, which is partially aromatic or substituted 9- or 10-membered heterobicycle, which is partially aromatic; X is selected from the group consisting of S, NH, or O; R² is selected from the group consisting of hydrogen, C₁₋₈allyl or C₁₋₈alkenyl; R³ and R⁴ are independently selected from the group consisting of hydrogen, hydroxy, oxo C₁₋₈alkyl, C₁₋₈ alkoxy or amino; R⁵ is selected from the group consisting of hydrogen, C₁₋₈alkyl, C₁₋₈alkenyl, (carbonyl)alkenyl, (hydroxy)alkenyl, phenyl, C₁₋₈alkyl; R⁶, (hydroxy) C₁₋₈alkyl; R⁶, C₁₋₈alkyl C₁₋₈cycloallyl; R⁶, (hydroxy) C₁₋C₁₋₈cycloallyl; R⁶ or C₁₋₈cycloallylthioR⁶; R⁶ is selected from the group consisting of phenyl or phenyl substituted with hydroxy, C₁₋₈alkyl or C₁₋₈alkoxy substituents; R⁷ is selected from the group consisting of hydrogen, hydroxy, carboxy or carboxy C₁₋₈alkyl; R⁸ is selected from the group consisting of hydrogen, C₁₋₈alkyl, phenyl, phenyl C₁₋₈alkyl, phenyl mono- or all-substituted with halo, hydroxy, and/or C₁₋₈alkoxy (e.g., methoxy) substituents or phenyl C₁₋₈alkyl wherein the phenyl is mono- or di-substituted with halo, hydroxy, and/or C₁₋₈alkoxy (e.g., methoxy) substituents; R⁹ is selected from the group consisting of hydrogen, C₁₋₈alkyl, carboxy C₁₋₈alkenyl mono- or dis-ubstituted with hydroxy, and/or C₁₋₈alkoxy (e.g., methoxy), phenyl or phenyl mono- or di-substituted with halo, hydroxy, and/or C₁₋₈alkoxy (e.g., methoxy); R¹⁰ is hydrogen or C₁₋₈alkyl; R¹¹ is selected from the group consisting of hydrogen, C₁₋₈alkyl or cycloC₁₋₈alkyl C₁₋₈alkyl; R¹² is selected from the group consisting of hydrogen, halo or fluorinated C₁₋₈alkyl; R¹³ is selected from the group consisting of hydrogen, C₁₋₈alkoxycarbonyl or C₁₋₈alkoxycarbonyl C₁₋₈alkylaminocarbonyl; a dashed line (- - -) is none or one double bond between two of the carbon atoms; fluorinated alkyl can be an alkyl wherein one or more of the hydrogen atoms is replaced by a fluorine atom; heteroaryl can be 5-, 6- or 7-membered aromatic ring optionally interrupted by 1, 2, 3 or 4 N, S, or O heteroatoms, with the proviso that any two O or S atoms are not bonded to each other; substituted heteroaryl can be a 9- or 10-membered heterobicycle mono-, di-, or tri-substituted independently with hydroxy, oxo, C₁₋₆ alkyl, C₁₋₆ alkoxy or 9- or 10-membered heterobicycle, which is partially aromatic in more detail is a heterobicycle interrupted by 1, 2, 3, or 4 N heteroatoms; substituted 9- or 10-membered heterobicycle, which is partially aromatic in more detail is a 9- or 10-membered heterobicycle mono-, di-, tri- or tetra-substituted independently with hydroxy, oxo, C₁₋₈ alkyl, C₁₋₈ alkoxy, phenyl, phenyl C₁₋₈ g alkyl; or a pharmaceutically acceptable acid-addition or base-addition salt thereof.

In yet other aspects, the PPARγ agonists can comprise a compound as disclosed in U.S. Pat. No. 6,306,854, e.g., a compound having a structure of Formula (XXVII):

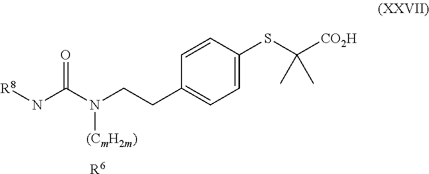

and esters, salts, and physiologically functional derivatives thereof; wherein m is from 0 to 20, R⁶ is selected from the group consisting of hydrogen and

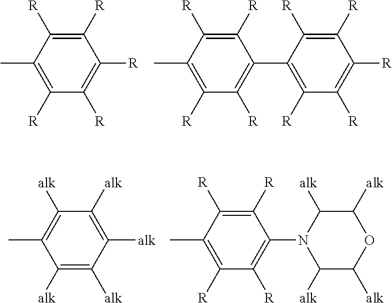

and R⁸ is selected frown the group consisting of:

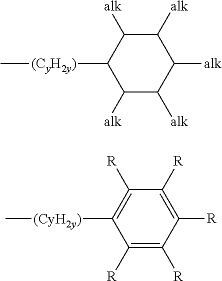

where y is 0, 1, or 2, each alk is independently hydrogen or alkyl group containing 1 to 6 carbon atoms, each R group is independently hydrogen, halogen, cyano, —NO₂, phenyl, straight or branched alkyl or fluoroalkyl containing 1 to 6 carbon atoms and which can contain hetero atoms such as nitrogen, oxygen, or sulfur and which can contain functional groups such as ketone or ester, cycloalkyl containing 3 to 7 carbon atoms, or two R groups bonded to adjacent carbon atoms can, together with the carbon atoms to which they are bonded, form an aliphatic or aromatic ring or multi ring system, and where each depicted ring has no more than 3 alk groups or R groups that are not hydrogen.

In yet other aspects of the present invention, a PPARγ agonist can comprise a compound such as those disclosed in U.S. Pat. No. 6,294,580 and/or Liu et al., Biorg. Med. Chem. Lett. 11 (2001) 3111-3113, e.g., having a structure within Formula XXVIII:

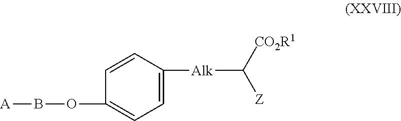

wherein A is selected from the group consisting of: (i) phenyl, wherein said phenyl is optionally substituted by one or more of the following groups; halogen atoms, C₁₋₆alkyl, C₁₋₃ alkoxy, C₁₋₃ fluoroalkoxy, nitrite, or —NR⁷R⁸ where R⁷ and R⁸ are independently hydrogen or C₁₋₃ alkyl; (ii) a 5- or 6-membered heterocyclic group containing at least one heteroatom selected from oxygen, nitrogen and sulfur; and (iii) a fused bicyclic ring:

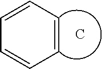

wherein ring C represents a heterocyclic group as defined in point (ii) above, which bicyclic ring is attached to group B via a ring atom of ring C; B is selected from the group consisting of: (iv) C₁₋₆ alkylene; (v) -M C₁₋₆ alkylene or C₁₋₆ alkyleneM C₁₋₆ alkylene, wherein M is O, S, or —NR² wherein R² represents hydrogen or C₁₋₃ alkyl; (vi) a 5- or 6-membered heterocyclic group containing at least one nitrogen heteroatom and optionally at least one further heteroaton selected from oxygen, nitrogen and sulfur and optionally substituted by C₁₋₃ alkyl; and (vii) Het-C₁₋₆ alkylene, wherein Het represents a heterocyclic group as defined in point (vi) above; Alk represents C₁₋₃ alkylene; Het represents hydrogen or C₁₋₃ alkyl; Z is selected from the group consisting of: (viii) nitrogen-containing heterocyclyl or heteroaryl, e.g., N-pyrrolyl, N-piperidinyl, N-piperazinyl, N-morpholinyl, or N-imidazolyl, optionally substituted with 1-4 C₁₋₆ alkyl or halogen substituents; (ix) —(C₁₋₃ alkylene) phenyl, which phenyl is optionally substituted by one or more halogen atoms; and (x) —NR³R⁴, wherein R³ represents hydrogen or C₁₋₃ alkyl, and R⁴ represents C₁₋₆ alkyl, aryl or heteroaryl (e.g., phenyl, pyridinyl, pyrazinyl, pyrimidinyl, pyrrolyl, piperidinyl, piperazinyl, morpholinyl, imidazolyl), optionally substituted by 1-4 C₁₋₆ alkyl, halogen, C₁₋₆ alkoxyl, hydroxyl, nitro, cyano, or amino substituents, or —Y—(C═O)-T-R⁵—Y—SO₂—R⁵, or —Y—(CH(OH))-T-R⁵, wherein: (a) Y represents a bond, C₁₋₆ alkylene, C₂₋₆ alkenylene, C₄₋₆ cycloalkylene or cycloalkenylene, a heterocyclic group as defined in point (vi) above, or phenyl optionally substituted by one or more C₁₋₃ alkyl groups and/or one or more halogen atoms; (b) T represents a bond, C₁₋₃ alkyleneoxy, —O— or —N(R⁶)—, wherein R⁵ represents hydrogen or C₁₋₃ alkyl; (c) R⁵ represents C₁₋₆ alkyl, C₄₋₆ cycloalkyl or cycloalkenyl, phenyl (optionally substituted by one or more of the following groups; halogen atoms, C₁₋₃ alkyl, C₁₋₃ alkoxy groups, C₁₋₃ alkyleneNR⁹R¹⁰ (where each R⁹ and R¹⁰ is independently hydrogen, C₁₋₃ alkyl, —SO₂ C₁₋₃ alkyl, or —CO₂ C₁₋₃ alkyl, —SO₂ NH C₁₋₃ alkyl), C₁₋₃ alkyleneCO₂H, C₁₋₃alkyleneCO₂C₁₋₃ alkyl, or —OCH₂C(O)NH₂), a 5- or 6-membered heterocyclic group as defined in point (ii) above, a bicylic fused ring:

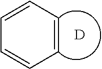

wherein ring D represents a 5- or 6-membered heterocyclic group containing at least one heteroatom selected from oxygen, nitrogen and sulfur and optionally substituted by (═O), which bicyclic ring is attached to T via a ring atom of ring D: or —C₁₋₆ alkyleneMR¹¹ M is O, S, or —NR¹² wherein R¹¹ and R¹² are independently hydrogen or C₁₋₃ alkyl, or a tautomeric form thereof, and/or a pharmaceutically acceptable salt or solvate thereof.

One specific group of compounds are those of Formula XI, wherein the dotted line represents no bond, R¹ is methyl, X is O and A is O. Examples of compounds in this group are those compounds where R is phenyl, 2-naphthyl and 3,5-bis(trifluoronethyl)phenyl. Another specific group of compounds are those of Formula XIII, wherein the dotted line represents no bond, R¹ is methyl and A is O. Particularly preferred compounds within this group are compounds where B is CH and R is phenol, p-tolyl, m-tolyl, cyclohexyl, and 2-naphthyl. In alternative embodiments of the present invention, the B is N and R is phenyl.

Specific examples of PPARγ agonist compounds of the present invention are given in the following list: (+)-5[[4-[(3,4-dihydro-6-hydroxy-2,5,7,8-tetramethyl-2H-1-benzopyran-2-yl)methoxy]phenyl]methyl]-2,4thiazolidinedione; (troglitazone); 5-[4-[2-(5-ethylpyridin-2-yl)ethoxyl]benzyl]thiazolidine-2,4-dione; (pioglitazone); 5-[4-[(1-methylcyclohexyl)methoxy]benzyl]thiazolidine-2,4-dione; (ciglitazone); 4-(2-naphthylmethyl)-1,2,3,5-oxathiadiazole-2-oxide; 5-[4-[2-[(N-(benzoxazol-2-yl)-N-methylamino]ethoxy]benzyl]-5-methlthiazolidine-2,4-dione; 5-[4-[2-[2,4dioxo-5-phenylthiazolidine-3-yl)ethoxy]benzyl]thiazolidine-2,4-dione; 5-[4-[2-[(N-methyl-N(phenoxycarbonyl)amino]ethoxy]benzyl]thiazolidine-2,4-dione; 5-[4-[2-phenoxyethoxy)benzyl]thiazolidine-2,4-dione; 5-[4-[2-(4-chorophenyl)ethylsulfonyl]benzyl]thiazolidine-2,4-dione; 5-[4-[3-(5-methyl-2-phenyloxazol-4-yl)propionyl]benzyl]thiazolidine-2,4-dione; 5-[[4-(3-hydroxyl-methylcyclohexyl)methoxy]benzyl]thiazolidine-2,4-dione; 5-[4-[2-(5-methyl-2-phenyloxazol-4-yl)ethoxyl]benzyl]thiazolidine-2,4-dione; 5-[(2-benzyl-2,3-dihydrobenzopyran)-5-ylmethyl]thiazolidine-2,4-dione; (englitazone); 5-[[2-(2-naphthylmethyl)benzoxazol]-5-ylmethyl]thiazolidine-2,4-dione; 5-[4-[2-(3-phenylureido)ethoxyl]benzyl]thiazolidine-2,4-dione; 5-[4-[2-(N-benzoxazol-2-yl)-N-metholamino]ethoxy]benzyl]thiazolidine-2,4-dione; 5-[4-[3-(5-methyl-2-phenyloxazol-4-yl)propionyl]benzyl]thiazolidine-2,4-dione; 5-[2-(5-methyl-2-phenyloxazol-4-ylmethyl)benzofuran-5-ylmethyl]oxazolidine-2,4-dione; 5-[4-[2-(N-methyl-N-(2-pyridyl)amino]ethoxy]benzyl]thiazolidine-2,4-dione (rosiglitazone); and 5-[4-[2-(N-(benzoxazol-2-yl)-N-methylamino]ethoxy]benzyl]oxazolidine-2,4-dione.

In yet other aspects of the present invention, the PPARγ agonists can comprise compounds having the structure shown in Formula XXIX:

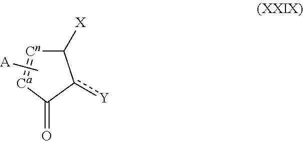

wherein: A is selected from hydrogen or a leaving group at the α- or β-position of the ring, or A is absent when there is a double bond between the Ca and Cn of the ring; X is an alkyl, substituted alkyl, alkenyl, substituted alkenyl, alkynyl, or substituted alkynyl group having in the range of 2 up to 15 carbon atoms; and Y is an alkyl, substituted alkyl, alkenyl, substituted alkenyl, alkynyl, or substituted alkynyl group having in the range of 2 up to 15 carbon atoms. As used herein, the term “leaving group” refers to functional groups which can readily be removed from the precursor compound, for example, by nucleophilic displacement, under E2 elimination conditions, and the like. Examples include, but are limited to, hydroxy groups, alkoxy groups, tosylates, brosylates, halogens, and the like.

Optionally, an LXR agonist can be administered in combination with a PPARγ agonist and a RXR agonist as described above. LXR agonists that can be used for practicing the present invention, and methods of making these compounds, are disclosed in PCT WO/03082198A2. In one aspect of the invention, the LXR agonists are selected from those disclosed in International Patent Applications WO 01154759 (Tularik Inc. US), PCT/US01127622 (SmithKline Beecham plc UK), WO 01141704 (Merck & CO., INC) and WO97/28137 (Merck & CO., INC).

In some aspects, the LXR agonist comprises a compound disclosed in International Patent Application WO 00/54759 having the following general formula (XXX):

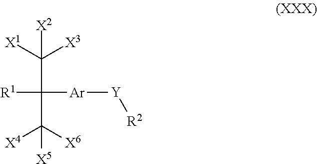

wherein:

Ar represents an aryl group;

R¹ is —OH, —O—(C₁-C₇)alkyl, —OC(O)—(C₁-C₇)alkyl, —O—(C₁-C₇)heteroalkyl, —OC(O)— (C₁-C₇) heteroalkyl, —CO₂H, —NH₂, —NH(C₁-C₇)alkyl, —N((C₁-C₇)alkylh or —NH—S(Oh-(C₁-C₈)alkyl;

R² is (C₁-C₇)alkyl, (C₁-C₇)heteroalkyl, aryl and aryl(C₁-C₇)alkyl;

X¹, X², X³, X⁴, X⁵ and X⁶ are each independently H, (CI-C₅)alkyl, (C₁-C₅)hetroalkyl, F or Cl, with the proviso that no more than three of X¹ through X⁶ are H, (C₁-C₅)alkyl or (C₁-C₅)heteroalkyl; and Y is —N(R¹²)S(O)m-, —N(R¹²)S(O)ₘN(R¹³)—, —N(R¹²)C(O), —N(R¹²)C(O)N(R¹³)—, —N(R¹²)C(S)— or —N(R¹²)C(O)O—, wherein R¹² and R¹³ are each independently hydrogen, (C₁-C₇)aryl, (C₁-C₇)heteroalkyl, aryl and aryl(C₁-C₇)alkyl, and optionally when Y is —N(R¹²)S(O)m- or —N(R¹²)S(O)mN(R¹³)—, R¹² forms a five, six or seven-membered ring fused to Ar or to R2 through covalent attachment to Ar or R2, respectively. In the above Y groups, the subscript m is an integer of from 1 to 2, as being useful as agonists of LXR and their use in pharmaceutical formulations of the present invention.

In some aspects the LXR agonist can include a compound with the following structure:

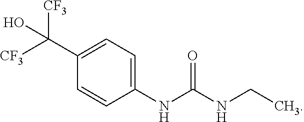

International Patent Application PCT/US01/27622 (SmithKline Beecham) discloses compounds of formula (XXXI):

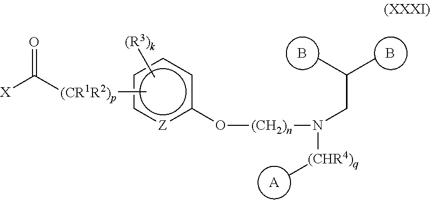

wherein:

X is OH or NH₂;

p is 0-6;

each R¹ and R² are the same or different and are each independently selected from the group consisting of H, C1-8alkyl, C1-8alkoxy and C1-8thioalkyl;

Z is CH or N; when Z is CH, k is 0-4; when Z is N, k is 0-3;

each R³ is the same or different and is independently selected from the group consisting of halo, —OH, C₁₋₈alkyl, C2-8alkenyl, C1-8alkoxy, C2-8alkenyloxy, —S(O)aR6, —NR7Rs, COR6, COOR6, R10COOR6, OR10COOR6, CONR7R8, —OC(O)R9, —R10NR7R8, —OR10NR7R8, 5-6 membered heterocycle, nitro, and cyano; a is 0, 1 or 2;

R⁶ is selected from the group consisting of H, C1-8 alkyl, C1-8 alkoxy and C2-8 alkenyl; each R⁷ and R⁸ are the same or different and are each independently selected from the group consisting of H, C1-8 alkyl, C2-8 alkenyl, C3-8 alkynyl;

R⁹ is selected from the group consisting of H, C1-8 alkyl and —NR7R8;

R¹⁰ is C1-6 alkyl;

n is 2-8;

q is 0 or 1;

R⁴ is selected from the group consisting of H, C1-8 alkyl, C1-8 alkenyl, and alkenyloxy;

Ring A is selected from the group consisting of C3-8 cycloalkyl, aryl, 4-8 membered heterocycle, and 5-6 membered heteroaryl;

each ring B is the same or different and is independently selected from the group consisting of C3-8 cycloalkyl and aryl.

In some aspects of the present invention, the LXR agonists can comprise 2-(3-{3-[[2-Chloro-3-(trifluoromethyl)benzyl](2,2-diphenylethyl)amino]propoxy}-phenyl)acetic acid, having the following structure:

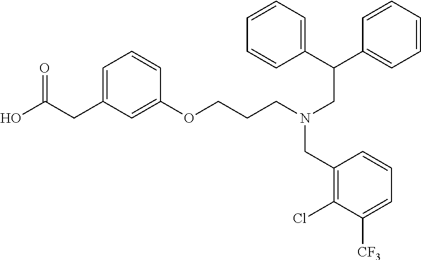

In some aspects of the present invention, the LXR agonists can comprise compounds of formula (XXXII), described in U.S. Provisional application Ser. Nos. 09/368,427, 60/368,425 and 60/368,426, each filed Mar. 27, 2002:

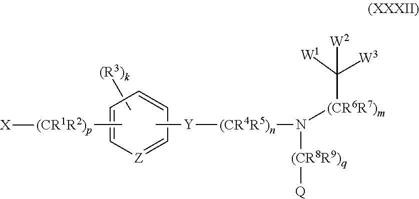

wherein: X is selected from C₁-C₈ alkyl, halo, —OR¹⁰, —NR¹⁴R¹⁵, nitro, cyano, —COOR¹⁰, —COR¹³, —OCOR¹³, —CONR¹⁴R¹⁵, —N(R¹⁷)COR¹³, —N(R¹⁷)CONR¹⁴R¹⁵, —N(R¹⁷)COOR¹³, —SO₃R, —SO₂NR¹⁴R¹⁵, —C(═NR¹⁷)NR¹⁴R¹⁵, —N(R¹⁷)SO₂R¹⁶, and a 5 or a 6-membered heterocyclic group;

or X and an adjacent R³, taken together with the atoms to which they are bonded, form an alkylenedioxy moiety;

Z is CH, CH3 or N, wherein when Z is CH or CH3, k is 0-4 and t is 0 or 1, and when

Z is N, k is 0-3 and t is 0;

Y is selected from —O—, —S—, —N(R20)-, and —C(R4)(R5)-;

W1 is selected from C¹-C₆ alkyl, C₃-C₈ cycloalkyl, aryl and Ret, wherein said C₁-C₆ alkyl, C₃-C₈ cycloalkyl, Ar and Ret are optionally unsubstituted or substituted with one or more groups independently selected from halo, cyano, nitro, C₁-C₆ alkyl, C₃-C₆ alkenyl, C₃-C₆ alkynyl, —C₀-C₆ alkyl-CO₂R¹⁰, —C₀-C₆ alkyl-C(O)SR₁₀, —C₀-C₆ alkyl-CONR₁₁R₁₂, —C₀-C₆ alkyl-COR₁₃, —CO—C₆ alkyl-NR₁₁R₁₂, —C₀-C₆ alkyl-SR₁₀, —C₀-C₆ alkyl-SR₁₀, —C₀-C₆ alkyl-SO₃H, —C₀-C₆ alkyl SO₂NR₁₁R₁₂, —C₀-C₆ alkyl-SO₂R₁₀, —C₀-C₆ alkyl-SOR₁₃, —C₀-C₆ alkyl-OCOR₁₂, —C₀-C₆ alkyl-OC(O)NR₁₁R₁₂, —C₀-C₆ alkyl-OC(O)OR₁₃, —C₀-C₆ alkyl-NR₁₁C(O)OR₁₃, —C₀-C₆ alkyl-NR₁₁C(O)NRI₁R₁₂, and —C₀-C₆ alkyl-NR₁₁COR₁₃, where said C₁-C₆alkyl, is optionally unsubstituted or substituted by one or more halo substituents;

W² is selected from R, halo, C₁-C₆ alkyl, C₂-C₆ alkenyl, C₂-C₆ alkynyl, —C₀-C₆ alkyl-NR₁₁R₁₂, —C₀-C₆ alkyl-SR₁₀, —C₀-C₆ alkyl-OR₁₀, —C₀-C₆ alkyl-CO₂R₁₀, —C₀-C₆ alkyl-C(O)SR₁₀, —C₀-C₆ alkyl-CONR₁₁R₁₂, —C₀-C₆ alkyl-COR₁₃, —C₀-C₆ alkyl-OCOR₁₃, —C₀-C₆ alkyl-OCONR₁₁R₁₂, —C₀-C₆ alkyl-NR₁₁CONR₁₁R₁₂, —C₀-C₆ alkyl-NR₁₁COR₁₃, —C₀-C₆ alkyl-Ret, —C₀-C₆ alkyl-Ar and —C₀-C₆ alkyl-C₃-C₇ cycloalkyl, wherein said C₁-C₆ alkyl is optionally unsubstituted or substituted by one or more halo substituents, and wherein the C₃-C₇ cycloalkyl, Ar and Ret moieties of said —CO—C₆ alkyl-Ret, —CO—C₆ alkyl-Ar and —CO—C₆ alkyl-C₃-C₇ cycloalkyl are optionally unsubstituted or substituted with one or more groups independently selected from halo, cyano, nitro, C₁-C₆ alkyl, C₃-C₆ alkenyl, C₃-C₆ alkynyl, —C₀-C₆ alkyl-CO₂R₁₀, —C₀-C₆ alkyl-C(O)SR₁₀, —C₀-C₆ alkyl-CONR₁₁R₁₂, —C₀-C₆ alkyl-COR₁₃, —C₀-C₆ alkyl-NR₁₁R₁₂, —C₀-C₆ alkyl-SR₁₀, —C₀-C₆ alkyl-OR₁₀, —C₀-C₆ alkyl-SO₃H, —C₀-C₆ alkyl-SO₂NR₁₁R₁₂, —CO—C₆ alkyl-SO₂R₁₀, —CO—C₆ alkyl-SOR₁₃, —CO—C₆ alkyl-OCOR₁₃, —C₀-C₆ alkyl-OC(O)NR₁₁R₁₂, —C₀-C₆ alkyl-OC(O)OR₁₃, —C₀-C₆ alkyl-NR₁₁C(O)OR₁₃, —C₀-C₆ alkyl-NR₁₁C(O)NR₁₁R₁₂, and —C₀-C₆ alkyl-NR₁₁COR₁₃, where said C₁-C₆ alkyl, is optionally unsubstituted or substituted by one or more halo substituents;

W3 is selected from the group consisting of: R, halo, C₁-C₆ alkyl, —C₀-C₆ alkyl-NR₁₁R₁₂, —C₀-C₆ alkyl-SR₁₀, —C₀-C₆ alkyl-OR₁₀, —C₀-C₆ alkyl-CO₂R₁₀, —C₀-C₆ alkyl-C(O)SR₁₀, —C₀-C₆ alkyl-CONR₁₁R₁₂, —C0-C₆ alkyl-COR₁₃, —C₀-C₆ alkyl-OCOR₁₃, —C₀-C₆ alkyl-OCONR₁₁R₁₂, —C₀-C₆ alkyl-NR₁₁CONR₁₁R₁₂, —C₀-C₆ alkyl-NR₁₁COR₁₃, —C₀-C₆ alkyl-Het, —C₁-C₆ alkyl-Ar and —C₁-C₆ alkyl-C₃-C₇ cycloalkyl, wherein said C₁-C₆ alkyl is optionally unsubstituted or substituted by one or more halo substituents;

Q is selected from C₃-C8 cycloalkyl, Ar and Het; wherein said C₃-C₈ cycloalkyl, Ar and Ret are optionally unsubstituted or substituted with one or more groups independently selected from halo, cyano, nitro, C₁-C₆ alkyl, C₃-C₆ alkenyl, C₃-C₆ alkynyl, —C₀-C₆ alkyl-CO₂R₁₀, —C₀-C₆ alkyl-C(O)SR₁₀, —C₀-C₆ alkyl-CONR₁₁R₁₂, —C₀-C₆ alkyl-COR₁₃, —C₀-C₆ alkyl-NR₁₁R₁₂, —C₀-C₆ alkyl-SR₁₀, —C₀-C₆ alkyl-OR₁₀, —C₀-C₆ alkyl-SO₃H, —C₀-C₆ alkyl-SO₂NR₁₁R₁₂, —C₀-C₆ alkyl-SO₂R₁₀, —C₀-C₆ alkyl-SOR₁₃, —C₀-C₆ alkyl-OCOR₁₃, —C₀-C₆ alkyl-OC(O)NR₁₁R₁₂, —C₀-C₆ alkyl-OC(O)OR₁₃, —C₀-C₆ alkyl-NR₁₁C(O)OR₁₃, —C₀-C₆ alkyl-NR₁₁C(O)NR₁₁R₁₂, and —C₀-C₆ alkyl-NR₁₁COR₁₃, where said C₁-C₆ alkyl is optionally unsubstituted or substituted by one or more halo substituents;

p is 0-8;

n is 2-8;

m is 0 or 1;

q is 0 or 1;

t is 0 or 1;

each R₁ and R₂ are independently selected from R, halo, C₁-C₆ alkyl, C₃-C₆ alkenyl, C₃-C₆ alkynyl, —C₀-C₆ alkyl-NR₁₁R₁₂, —C₀-C₆ alkyl-OR₁₀, —C₀-C₆ alkyl-SR₁₀, —C₁-C₆ alkyl-Het, —C₁-C₆ alkyl-Ar and —C₁-C₆ alkyl-C₃-C₇ cycloalkyl, or R₁ and R₂ together with the carbon to which they are attached form a 3-5 membered carbocyclic or heterocyclic ring, wherein said heterocyclic ring contains one, or more heteroatoms selected from N, O, and S, where any of said C₁-C₆ alkyl is optionally unsubstituted or substituted by one or more halo substituents; each R₃ is the same or different and is independently selected from halo, cyano, nitro, C₁-C₆ alkyl, C₃-C₆ alkenyl, Cr C₆ alkynyl, —C₀-C₆ alkyl-Ar, —C₀-C₆ alkyl-Het, —C₀-C₆ alkyl-C₃-C₇ cycloalkyl, —C0-C₆ alkyl-CO₂R₁₀, —C₀-C₆ alkyl-C(O)SR₁₀, —C₀-C₆ alkyl-CONR₁₁R₁₂, —C₀-C₆ alkyl-COR₁₃, —C₀-C₆ alkyl-NR₁₁R₁₂, —C₀-C₆ alkyl-SR₁₀, —C₀-C₆ alkyl-OR₁₀, —C₀-C₆ alkyl-SO₃H, —CO—C₆ alkyl-SO₂NR₁₁R₁₂, —C₀-C₆ alkyl-SO₂ R₁₀, —C₀-C₆ alkyl-SOR₁₃, —C₀-C₆ alkyl-OCOR₁₃, —C₀-C₆ alkyl-OC(O)NR₁₁R₁₂, —C₀-C₆ alkyl-OC(O)OR₁₃, —CO—C₆ alkyl-NR₁₁C(O)OR₁₃, —CO—C₆ alkyl-NR₁₁C(O)NR₁₁R₁₂, and —CO—C₆ alkyl-NR₁₁ COR₁₃, wherein said C₁-C₆ alkyl is optionally unsubstituted or substituted by one or more halo substituents; each R₄ and R₅ is independently selected from H, halo, C₁-C₆ alkyl, —C₀-C₆ alkyl-Het, —C₀-C₆ alkyl-Ar and —C₀-C₆ alkyl-C₃-C₇ cycloalkyl; R₆ and R₇ are each independently selected from H, halo, C₁-C₆ alkyl, —C₀-C₆ alkyl-Het, —C₀-C₆ alkyl-Ar and —C₀-C₆ alkyl-C₃-C₇ cycloalkyl; R₈ and R₉ are each independently selected from H, halo, C₁-C₆ alkyl, —C₀-C₆ alkyl-Het, —C₀-C₆ alkyl-Ar and —C₀-C₆ alkyl-C₃-C₇ cycloalkyl; R₁₀ is selected from H, C₁-C₆ alkyl, C₃-C₆ alkenyl, C₃-C₆ alkynyl, —C₀-C₆ alkyl-Ar, —C₀-C₆ alkyl-Het and —C₀-C₆ alkyl-C₃-C₇ cycloalkyl; each R₁₁ and each R₁₂ are independently selected from H, C₁-C₆ alkyl, C₃-C₆ alkenyl, C₃-C₆ alkynyl, —C₀-C₆ alkyl-Ar, —C₀-C₆ alkyl-Het and —C₀-C₆ alkyl-C₃-C₇ cycloalkyl, or R₁₁ and R₁₂ together with the nitrogen to which they are attached form a 4-7 membered heterocyclic ring which optionally contains one or more additional heteroatoms selected from N, O, and S; R₁₃ is selected from C₁-C₆ alkyl, C₃-C₆ alkenyl, C₃-C₆ alkynyl, —C₀-C₆ alkyl-Ar, —C₀-C₆ alkyl-Het and —C₀-C₆ alkyl-C₃-C₇ cycloalkyl;

R₁₄ and R₁₅ are each independently selected from H, C₁-C₆ alkyl, C₃-C₆ alkenyl, C₃-C₆ alkynyl, —C₀-C₆ alkyl-Ar, —C₀-C₆ alkyl-Het, —C₀-C₆ alkyl-C₃-C₇ cycloalkyl, —C₀-C₆ alkyl-O—Ar, —C₀-C₆ alkyl-O-Het, —C₀-C₆ alkyl-O—C₃-C₇ cycloalkyl, —C₀-C₆ alkyl-S(O)x-C₁-C₆ alkyl, —C₀-C₆ alkyl-S(O)x—Ar, —C₀-C₆ alkyl-S(O)x-Het, —C₀-C₆ alkyl-S(O)x-C₃-C₇ cycloalkyl, —C₀-C₆ alkyl-NH-Het, —C₀-C₆ alkyl-NH—C₃-C₇ cycloalkyl, —C₀-C₆ alkyl-N(C₁-C₄ alkyl)-Ar, —C₀-C₆ alkyl-N(C₁-C₄ alkyl)-Het, —C₀-C₆ alkyl-N(C₁-C₄ alkyl)-C₃-C₇cycloalkyl, —C₀-C₆ alkyl-Ar, —C₀-C₆ alkyl-Het and —C₀-C₆ alkyl-C₃-C₇ cycloalkyl, where x is 0, 1 or 2, or R₁₄ and R₁₅, together with the nitrogen to which they are attached, form a 4-7 membered heterocyclic ring which optionally contains one or more additional heteroatoms selected from N, O, and S, wherein said C₁-C₆ alkyl is optionally substituted by one or more of the substituents independently selected from the group halo, —OH, —SH, —NH₂, —NH(unsubstituted C₁-C₆ alkyl), —N(unsubstituted C₁-C₆ alkyl)(unsubstituted C₁-C₆ alkyl), unsubstituted —OC₁-C₆ alkyl, —CO₂H, —CO₂(unsubstituted C₁-C₆ alkyl), —CONH₂, CONH(unsubstituted C₁-C₆ alkyl), —CON(unsubstituted C₁-C₆ alkyl)(unsubstituted C₁-C₆ alkyl), —SO₃H, —SO₂NH₂, —SO₂NH(unsubstituted C₁-C₆ alkyl) and —SO₂N(unsubstituted C₁-C₆ alkyl)(unsubstituted C₁-C₆ alkyl);

R₁₆ is C₁-C₆ alkyl, —C₁-C₆ alkyl-Ar or —C₀-C₆ alkyl-Het; and

R₁₇ is H, C₁-C₆ alkyl, —C₀-C₆ alkyl-Ar or —C₀-C₆ alkyl-Het; or a pharmaceutically acceptable salt or solvate thereof.

In some aspects of the present invention, the LXR agonist can include N-(2,2,2-trifluoroethyl)-N-[4-(2,2,2-trifluoro-1-hydroxy-1-trifluoromethylethyl)-phenyl]-benzenesulfonamide (also known as T0901317) having the following chemical structure:

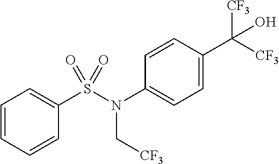

Other examples of suitable LXR agonists for use in the present invention include: (R)-2-(3-{3-[[2-Chloro-3-(trifluoromethyl)benzyl](2,2diphenylethyl)amino]-1-methyl-propoxy}-phenyl)acetic acid methyl ester

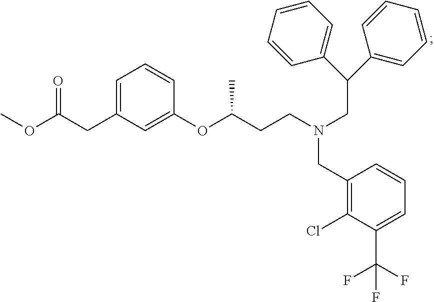

### (2-Chloro-3-trifluoromethyl-benzyl)-(2,2-diphenyl-ethyl)-{3-[3-(1,2,3,4tetrazol-5-ylmethyl)-phenoxy]-propyl}-amine

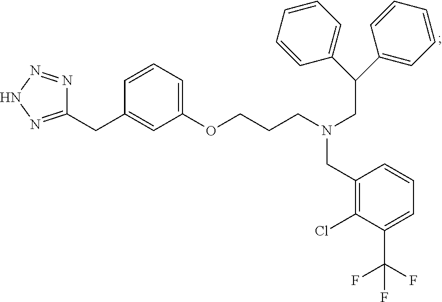

### (S)-2-(3-{3-[[2-Chloro-3-(trifluoromethyl)benzyl](2-phenylpropyl)amino]propoxy}-phenyl)-acetamide

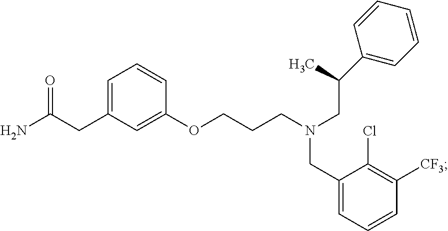

and

### 2-{2-[[2-Chloro-3-(trifluoromethyl)benzyl](2,2-diphenylethyl)amino]-ethyl}-6-benzofuran acetic acid hydrochloride

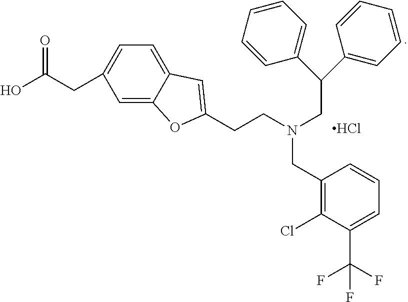

Additional LXR agonists useful in the methods of the present invention include those of Formula (XXXIII), which are described in U.S. Provisional Application No. 60/368,415, filed Mar. 27, 2002:

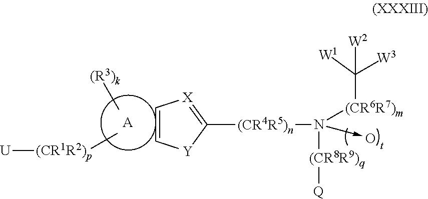

wherein:

X is CH or N;

Y is N(R¹⁰), O, or S, wherein t is 0 or 1 when Y is N(R¹⁰) or 0, and t is 0 when Y is S;

U is selected from halo, —OR¹⁰, —NR¹⁴R¹⁵, nitro, cyano, —COOR¹⁰, —COR¹³, —OCOR¹³, —CONR¹⁴R¹⁵, —N(R¹⁴)COR¹³, —SO₃H, —SO₂NR¹⁴R¹⁵, —C(═NR¹⁷)NR¹⁴R¹⁵, —N(R¹⁴)SO₂R¹⁶, and a 5 or 6-membered heterocyclic group;

A is a phenyl fused ring moiety or a pyridyl fused ring moiety, wherein when A is a phenyl ring moiety, k is 0-3 and t is 0 or 1 and when A is a pyridyl ring moiety, k is 0-2 and t is 0;

W¹ is selected from C₃-C₈ cycloalkyl, aryl and Het, wherein said C₃-C₈ cycloalkyl, Ar and Het are optionally unsubstituted or substituted with one or more groups independently selected from halo, cyano, nitro, C₁-C₆ alkyl, C₃-C₆ alkenyl, C₃-C₆ alkynyl, —C₀-C₆ alkyl-CO₂R₁₀, —C₀-C₆ alkyl-C(O)SR₁₀, —CO—C₆ alkyl-CONR₁₁R₁₂, —C₀-C₆ alkyl-C₀R₁₃, —C₀-C₆ alkyl-NR₁₁R₁₂, —C₀-C₆ alkyl-SR₁₀, —C₀-C₆ alkyl-OR₁₀, —C₀-C₆ alkyl-SO₃H, —C₀-C₆ alkyl-SO₂NR₁₁R₁₂, —C₀-C₆ alkyl-SO₂R₁₀, —C₀-C₆ alkyl-SOR₁₃, —C₀-C₆ alkyl-OCOR₁₃, —C₀-C₆ alkyl-OC(O)NR₁₁R₁₂, —C₀-C₆ alkyl-OC(O)OR₁₃, —C₀-C₆ alkyl-NR₁₁C(O)OR₁₃, —C₀-C₆ alkyl-NR₁₁C(O)NR₁₁R₁₂, and —C₀-C₆ alkyl-NR₁₁COR₁₃, where said C₁-C₆ alkyl, is optionally unsubstituted or substituted by one or more halo substituents;

W₂ is selected from H, halo, C₁-C₆ alkyl, C₂-C₆ alkenyl, C₂-C₆ alkynyl, —C₀-C₆ alkyl-NR₁₁R₁₂, —C₀-C₆ alkyl-SR₁₀, —C₀-C₆ alkyl-OR₁₀, —C0-C₆ alkyl-CO₂R₁₀, —C₀-C₆ alkyl-C(O)SR₁₀, —C₀-C₆ alkyl-CONR₁₁R₁₂, —C₀-C₆ alkyl-COR₁₃, —C₀-C₆ alkyl-OCOR₁₃, —C0-C₆ alkyl-OCONR₁₁R₁₂, —C₀-C₆ alkyl-NR₁₁CONR₁₁R₁₂, —C₀-C₆ alkyl-NR₁₁COR₁₃, —C₀-C₆ alkyl-Het, —C₀-C₆ alkyl-Ar and —C₀-C₆ alkyl-C₃-C₇ cycloalkyl, wherein said C₁-C₆ alkyl is optionally unsubstituted or substituted by one or more halo substituents, and wherein the C₃ C₇ cycloalkyl, Ar and Het moieties of said —C₀-C₆ alkyl-Het, —C₀-C₆ alkyl-Ar and —C₀-C₆ alkyl-C₃-C₇ cycloalkyl are optionally unsubstituted or substituted with one or more groups independently selected from halo, cyano, nitro, C₁-C₆ alkyl, C₃-C₆ alkenyl, C₃-C₆ alkynyl, —C₀-C₆ alkyl-CO₂R₁₀, —C₀-C₆ alkyl-C(O)SR₁₀, —C₀-C₆ alkyl-CONR₁₁R₁₂, —C₀-C₆ alkyl-COR₁₃, —C₀-C₆ alkyl-NR₁₁R₁₂, —C₀-C₆ alkyl-SR₁₀, —C₀-C₆ alkyl-OR₁₀, —C₀-C₆ alkyl-SO₃H, —C₀-C₆ alkyl-SO₂NR₁₁R₁₂, —C₀-C₆ alkyl-SO₂R₁₀, —C₀-C₆ alkyl-SOR₁₃, —C₀-C₆ alkyl-OCOR₁₃, —C₀-C₆ alkyl-OC(O)NR₁₁R₁₂, —C₀-C₆ alkyl-OC(O)OR₁₃, —C₀-C₆ alkyl-NR₁₁C(O)OR₁₃, —C₀-C₆ alkyl-NR₁₁C(O)NR₁₁R₁₂, and —C₀-C₆ alkyl-NR₁₁COR₁₃, where said C₁-C₆ alkyl, is optionally unsubstituted or substituted by one or more halo substituents;

W₃ is selected from the group consisting of: H, halo, C₁-C₆ alkyl, —C₀-C₆ alkyl-NR₁₁R₁₂, —C₀-C₆ alkyl-SR₁₀, —C₀-C₆ alkyl-OR₁₀, —C₀-C₆ alkyl-CO₂R₁₀, —C₀-C₆ alkyl C(O)SR₁₀, —C₀-C₆ alkyl-CONR₁₁R₁₂, —C₀-C₆ alkyl-COR₁₃, —C₀-C₆ alkyl-OCOR₁₃, —C₀-C₆ alkyl-OCONR₁₁R₁₂, —C₀-C₆ alkyl-NR₁₁CONR₁₁R₁₂, —C₀-C₆ alkyl-NR₁₁COR₁₃, —C₀-C₆ alkyl-Het, —C₁-C₆ alkyl-Ar and —C₁-C₆ alkyl-C₃-C₇ cycloalkyl, wherein said C₁-C₆ alkyl is optionally unsubstituted or substituted by one or more halo substituents;

Q is selected from C₃-C₈ cycloalkyl, Ar and Het; wherein said C₃-C₈ cycloalkyl, Ar and Het are optionally unsubstituted or substituted with one or more groups independently selected from halo, cyano, nitro, C₁-C₆ alkyl, C₃-C₆ alkenyl, C₃-C₆ alkynyl, —C₀-C₆ alkyl-CO₂R₁₀, —C₀-C₆ alkyl-C(O)SR₁₀, —C₀-C₆ alkyl CONR₁₁R₁₂, —C₀-C₆ alkyl-COR₁₃, —C₀-C₆ alkyl-NR₁₁R₁₂, —C₀-C₆ alkyl-SR₁₀, —CO—C₆ alkyl-OR₁₀, —C₀-C₆ alkyl-SO₃H, —C₀-C₆ alkyl-SO₂NR₁₁R₁₂, —C₀-C₆ alkyl-SO₂R₁₀, —C₀-C₆ alkyl-SOR₁₃, —C₀-C₆ alkyl-OCOR₁₃, —C₀-C₆ alkyl-OC(O)NR₁₁R₁₂, —C₀-C₆ alkyl-OC(O)OR₁₃, —C₀-C₆ alkyl-NR₁₁C(O)OR₁₃, —C₀-C₆ alkyl-NR₁₁C(O)NR₁₁R₁₂, and —C₀-C₆ alkyl-NR₁₁COR₁₃, where said C₁-C₆ alkyl is optionally unsubstituted or substituted by one or more halo substituents;

p is 0-8;

n is 2-8;

m is 0 or 1;

q is 0 or 1;

t is 0 or 1;

each R₁ and R₂ are independently selected from R, halo, C₁-C₆ alkyl, C₃-C₆ alkenyl, C₃-C₆ alkynyl, —C₀-C₆ alkyl-NR₁₁R₁₂, —C₀-C₆ alkyl-OR₁₀, —C₀-C₆ alkyl-SR₁₀, —C₁-C₆ alkyl-Het, —C₁-C₆ alkyl-Ar and —C₁-C₆ alkyl-C₁-C₇ cycloalkyl, or R₁ and R₂ together with the carbon to which they are attached form a 3-5 membered carbocyclic or heterocyclic ring, wherein said heterocyclic ring contains one, or more heteroatoms selected from N, O, and S, where said C₁-C₆ alkyl is optionally unsubstituted or substituted by one or more halo substituents;

each R₃ is the same or different and is independently selected from halo, cyano, nitro, C₁-C₆ alkyl, C₁-C₆ alkenyl, C₁-C₆ alkynyl, —C₀-C₆ alkyl-Ar, —C₀-C₆ alkyl-Het, —C₀-C₆ alkyl-C₃-C₇ cycloalkyl, —C₀-C₆ alkyl-CO₂R₁₀, —C₀-C6 alkyl-C(O)SR₁₀, —C₀-C₆ alkyl-CONR₁₁R₁₂, —C₀-C₆ alkyl-COR₁₃, —C₀-C₆ alkyl-NR₁₁R₁₂, —C₀-C₆ alkyl-SR₁₀, —C₀-C₆ alkyl-OR₁₀, —C₀-C₆ alkyl-SO₃R, —C₀-C₆ alkyl-SO₂NR₁₁R₁₂, —C₀-C₆ alkyl-SO₂R₁₀, —C₀-C₆ alkyl-SOR₁₃, —C₀-C₆ alkyl-OCOR₁₃, —C₀-C₆ alkyl-OC(O)NR₁₁R₁₂, —C₀-C₆ alkyl-OC(O)OR₁₃, —C₀-C₆ alkyl-NR₁₁C(O)OR₁₃, —CO—C₆ alkyl-NR₁₁C(O)NR₁₁R₁₂, and —C₀-C₆ alkyl-NR₁₁COR₁₃, wherein said C₁-C₆ alkyl is optionally unsubstituted or substituted by one or more halo substituents;

each R₄ and R₅ is independently selected from R, halo, C₁-C₆ alkyl, —C₀-C₆ alkyl-Het, —C₀-C₆ alkyl-Ar and —C₀-C₆ alkyl-C₁-C₇ cycloalkyl;

R₆ and R₇ are each independently selected from R, halo, C₁-C₆ alkyl, —C₀-C₆ alkyl-Het, —C₀-C₆ alkyl-Ar and —C0-C₆ alkyl-C₃-C₇ cycloalkyl;

R₈ and R₉ are each independently selected from R, halo, C₁-C₆ alkyl, —C₀-C₆ alkyl-Het, —C₀-C₆ alkyl-Ar and —C₀-C₆ alkyl-C₃-C₇ cycloalkyl;

R₁₀ is selected from R, C₁-C₆ alkyl, C₁-C₆ alkenyl, C₃-C₆ alkynyl, —C₀-C₆ alkyl-Ar, —C₀-C₆ alkyl-Het and —C₀-C₆ alkyl-C₃-C₇ cycloalkyl;

each R₁₁ and each R₁₂ are independently selected from H, C₁-C₆ alkyl, C₃-C₆ alkenyl, C₃-C₆ alkynyl, —C₀-C₆ alkyl-Ar, —C₀-C₆ alkyl-Het and —C₀-C₆ alkyl-C₃-C₇ cycloalkyl, or R₁₁ and R₁₂ together with the nitrogen to which they are attached form a 4-7 membered heterocyclic ring which optionally contains one or more additional heteroatoms selected from N, O and S;

R₁₃ is selected from C₁-C₆ alkyl, C₃-C₆ alkenyl, C₃-C₆ alkynyl, —C₀-C₆ alkyl-Ar, —C₀-C₆ alkyl-Het and —C₀-C₆ alkyl-C₁-C₇ cycloalkyl;

R₁₄ and R₁₅ are each independently selected from H, C₁-C₆ alkyl, C₃-C₆ alkenyl, C₃-C₆ alkynyl, —C₀-C₆ alkyl-Ar, —C₀-C₆ alkyl-Het, —C₀-C₆ alkyl-C₃-C₇ cycloalkyl, —C₀-C₆ alkyl-O—Ar, —C₀-C₆ alkyl-O-Het, —C₀-C₆ alkyl-O—C₃-C₇ cycloalkyl, —C₀-C₆ alkyl-S(O)x-C₁-C₆ alkyl, —C₀-C₆ alkyl-S(O)xAr, —C₀-C₆ alkyl-S(O)xHet, —C₀-C₆ alkyl-S(O)xC₃-C₇ cycloalkyl, —C₀-C₆ alkyl-NH—Ar, —C₀-C₆ alkyl-NH-Het, —C₀-C₆ alkyl-NH—C₃-C₇ cycloalkyl, —C₀-C₆ alkyl-N(C₁-C₄ alkyl)-Ar, —C₀-C₆ alkyl-N(C₁-C₄ alkyl)-Het, —C₀-C₆ alkyl-N(C₁-C₄ alkyl)-C₃-C₇ cycloalkyl, —C₀-C₆ alkyl-Ar, —C₀-C₆ alkyl-Het and —C₁-C₆ alkyl-C₃-C₇ cycloalkyl, where x is 0, 1 or 2, or R₁₄ and R₁₅ are each independently selected from H, C₁-C₆ alkyl, C₃-C₆ alkenyl, C₃-C₆ alkynyl, —C₀-C₆ alkyl-Ar, —C₀-C₆ alkyl-Het, —C₀-C₆ alkyl-C₃-C₇ cycloalkyl, —C₀-C₆ alkyl-O—Ar, —CO—C₆ alkyl-O-Ret, —CO—C₆alkyl-0-C₃-C₇ cycloalkyl, —CO—C₆alkyl-S(0)x-C₁-C₆ alkyl, —CO—C₆alkyl-S(OkAr, —CO—C₆ alkyl-S(OkRet, —CO—C₆alkyl-S(OkC₃-C₇ cycloalkyl, —CO—C₆alkyl-NR—Ar, —CO—C₆ alkyl-NR-Ret, —CO—C₆ alkyl-NR—C₃-C₇ cycloalkyl, —CO—C₆alkyl-N(C₁-C₄alkyl)-Ar, —CO—C₆alkyl-N(C₁-C₄alkyl)-Ret, —CO—C₆alkyl-N(C₁-C₄alkyl)-C₃-C₇ cycloalkyl, —CO—C₆ alkyl-Ar, —CO—C₆ alkyl-Ret and —CO—C₆ alkyl-C₃-C₇ cycloalkyl, where x is 0, 1 or 2, or R₁₄ and R₁₅, together with the nitrogen to which they are attached, form a 4-7 membered heterocyclic ring which optionally contains one or more additional heteroatoms selected from N, O, and S, wherein said C₁-C₆ alkyl is optionally substituted by one or more of the substituents independently selected from the group halo, —OH, —SH, —NH2, NH(unsubstituted C₁-C₆ alkyl), —N(unsubstituted C₁-C₆ alkyl)(unsubstituted C₁-C₆ alkyl), unsubstituted —OC₁-C₆ alkyl, —CO₂H, —CO₂(unsubstituted C₁-C₆ alkyl), —CONH₂, —CONH(unsubstituted C₁-C₆ alkyl), —CON(unsubstituted C₁-C₆ alkyl)(unsubstituted C₁-C₆ alkyl), —SO₃H, —SO₂NH₂, —SO₂NH(unsubstituted C₁-C₆ alkyl) and —SO₂N(unsubstituted C₁-C₆ alkyl)(unsubstituted C₁-C₆ alkyl);

R₁₆ is C₁-C₆ alkyl, —C₀-C₆ alkyl-Ar or —C₀-C₆ alkyl-Het; and

R₁₇ is H, C₁-C₆ alkyl, —C₀-C₆ alkyl-Ar or —C₀-C₆ alkyl-Het; or a pharmaceutically acceptable salt or solvate thereof.

Unless otherwise provided, each alkyl, alkoxy, alkenyl, alkynyl, cycloalkyl, aryl or Het (including any 3-5-membered, 4-7-membered or 5-7-membered carbocyclic or heterocyclic rings or ring moieties) in the compounds of formula (2 generics above with W groups) is independently unsubstituted or substituted with one or more substituents defined herein below.

In the compounds of formula (2 generics directly above), group A is defined as a phenyl or a pyridyl fused ring moiety and is exemplified by the following: phenyl: pyridyl:

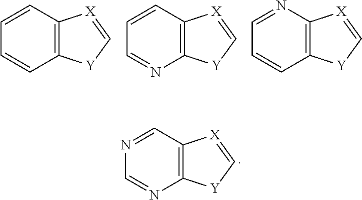

International Patent Application WO 01/41704 (Merck & Co., Inc.) discloses a compound of formula (XXXIV) and (XXXV):

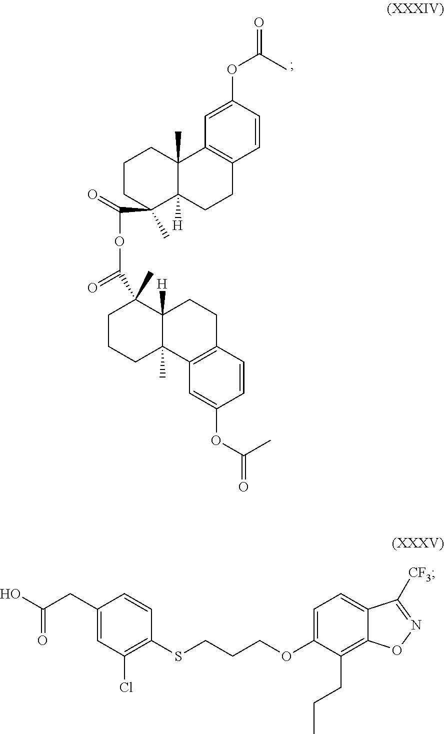

and related compounds alongside methods for their production as described in International Patent Application WO97/28137 (Merck & Co.), along with methods for making them.

The RXR agonists, PPARγ agonists, and the LXR agonists of the present invention described herein are capable of further forming both pharmaceutically acceptable acid addition and/or base salts.

Pharmaceutically acceptable acid addition salts of the present invention can include, but are not limited to, salts derived from nontoxic inorganic acids such as hydrochloric, nitric, phosphohoric, sulfuric, hydrobromic, hydriodic, hydrofluoric, phosphorous, and the like, as well as the salts derived forth nontoxic organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. Such salts thus include sulfate, pyrosulfate, bisulfate, sulfite, bissulfite, nitrate, phosphate, monoLydrogenphosphate, dihydrogenphosphate, metaphosphate, pyrophosphate, chloride, bromide, iodide, acetate, trifluoracetate, propionate, caprylate, isobutyrate, oxalate, malonate, succinate, suberate, sebacate, fumarate, malcate, mandelate, benzoate, chlorobenzoate, methylbenzoate, dinitrobenzoate, phthalate, benzenesulfonate, toluenesulfonate, phenylacetate, citrate, lactate, maleate, tartrate, methanesulfonate, and the like. Also contemplated are salts of amino acids such as arginate and the like, as well as gluconate, galacturonate, and n-methyl glucamine.

The acid addition salts of the basic compounds are prepared by contacting the free base form with a sufficient amount of the desired acid to produce the salt in the conventional manner. The free base form may be regenerated by contacting the salt form with a base and isolating the free base in the conventional manner or as described above. The free base forms differ from their respective salt forms somewhat in certain physical properties such as solubility in polar solvents, but are otherwise equivalent to their respective free base for purposes of the present invention.

Pharmaceutically acceptable base addition salts are formed with metals or amides, such as alkali and alkaline earth metals or organic amines. Examples of metals used as cations include, but are not limited to, sodium, potassium, magnesium, calcium, and the like. Examples of suitable amines include, but are not limited to, N2-N′-dibenzylethylenediamine, chloroprocaine, choline, diethanolamine, dicyclohexylamine, ethylenediamine, N-methylglucamine, and procaine.

The base addition salts of the acidic compounds are prepared by contacting the free acid form with a sufficient amount of the desired base to produce the salt in the conventional manner. The free acid form may be regenerated by contacting the salt form with an acid and isolating the free acid in the conventional manner or as described above. The free acid forms differ from their respective salt forms somewhat in certain physical properties such as solubility in polar solvents, but otherwise the salts are equivalent to their respective free acid for purposes of the present invention.

Certain of the compounds of the present invention can exist in unsolvated forms as well as solvated forms, including, but not limited to, hydrated forms. In general, the solvated forms, including hydrated forms, are equivalent to unsolvated forms and are intended to be encompassed within the scope of the present invention. Certain of the compounds of the present invention possess one or more chiral centers and each center may exist in different configurations. The compounds can, therefore, form stereoisomers. Although these are all represented herein by a limited number of molecular formulas, the present invention includes the use of both the individual, isolated isomers and mixtures, including racemates, thereof. Where stereo-specific synthesis techniques are employed or optically active compounds are employed as starting materials in the preparation of the compounds, individual isomers may be prepared directly. However, if a mixture of isomers is prepared, the individual isomers may be obtained by conventional resolution techniques, or the mixture may be used as is, with resolution.

The dose, amount, and/or quantity of the pharmaceutical compositions described above, which are administered to the subject can depend on the specific RXR agonists, PPARγ agonists, or optionally LXR agonists selected. It will be appreciated that the dosage amounts used will depend on the potency of the specific RXR agonists, PPARγ agonists, or the LXR agonists and the therapeutic regimen employed.

In another aspect, the PPARγ agonist and RXR agonist when administered in combination to subject can be administered at an amount or dosage to achieve a therapeutic effect that is substantially less (i.e., subtherapeutic dose or amount) than the amount or dose that would be required to achieve a therapeutic effect if each compound was administered alone. Co-administration of a PPARγ agonist and RXR agonist to the subject can also mitigate resistance to one single agent. Such resistance results either in the requirement for higher dosages of the drug and/or the renewed symptoms.

Moreover, co-administration of a PPARγ agonist and RXR agonist to the subject can mitigate toxicity and side effects associated with potentially administering a single agent at an amount effective to achieve a therapeutic effect. For example, according to an FDA alert issued on May 21, 2007, therapeutic doses of the PPARγ agonist rosiglitazone, are associated with a significantly increased risk of heart attack, and even higher risk of death from all cardiovascular diseases. In addition, both rosiglitazone and pioglitazone have been suspected of causing macular edema. Therefore, there is a practical upper limit to the amount that a subject can receive. However, if two or more agents are used in concert, the dosage of any single drug can be lowered. This is beneficial to the patient since using lower levels of therapeutic agents is generally safer for the patient. Additionally, cells are less likely to generate resistance to the combination of drugs as they are to a single drug. Thus in some aspects of the present invention, the compositions described herein can be administered to a subject at a subtherapeutic level.

The present invention is not limited by the order in which the agents are administered. In one embodiment, the agents are administered sequentially. In another embodiment, the agents are administered as a combined formulation (e.g., a formulation comprising a PPARγ agonist and an RXR agonist).

The PPARγ agonists, RXR agonists, and optionally LXR agonists can be formulated for systemic administration and/or topical administration. The PPARγ agonists, RXR agonists, and optionally LXR agonists of the present invention are not limited by the route of administration. Pharmaceutical compositions comprising the PPARγ agonists, RXR agonists, and optionally LXR agonists may be administered orally, intravenously, intraperitoneally. In some aspects of the present invention, pharmaceutical compositions may be administered directly to a lesion or injury site by injection or, in the case of dermatological disorders, for example, by direct application of creams or ointments. In certain aspects, one agent is administered by one route, while the second agent is administered by a second route.

Advantageously, the PPARγ agonists, RXR agonists, and optionally LXR agonists can be administered by local topical administration to the site of the dermatological disorder. Topical administration is desirable because a lower dosage can be administered to the subject being treated to provide a therapeutically effective benefit. Additionally, administration of a lower topical dosage can mitigate adverse side-effects that may be associated with systemic administration.

Topical formulations include those for delivery via the mouth (buccal) and through the skin such that at least one layer of skin (i.e., the epidermis, dermis, and/or subcutaneous layer) is contacted with a PPARγ agonists, RXR agonists, and optionally LXR agonists or derivative thereof. Topical delivery systems may be used to administer topical formulations of the present invention. Topical delivery systems can include, for example, transdermal patches containing a PPARγ agonists, an RXR agonists, and optionally an LXR agonists or derivative thereof to be administered. Delivery through the skin can further be achieved by iontophoresis or electrotransport, if desired.

Formulations for topical administration in the mouth can include any one or combination of: lozenges comprising a PPARγ agonists, RXR agonists, and optionally LXR agonists or derivative thereof in a flavored basis, usually sucrose and acacia or tragacanth; pastilles comprising a PPARγ agonists, RXR agonists, and optionally LXR agonists or derivative thereof in an inert basis such as gelatin and glycerin or sucrose and acacia; and mouthwashes comprising a PPARγ agonists, RXR agonists, and optionally LXR agonists or derivative thereof to be administered in a suitable liquid carrier.

Formulations for topical administration to the skin can include ointments, creams, gels, and pastes comprising PPARγ agonists, RXR agonists, and optionally LXR agonists or derivatives thereof to be administered in a pharmaceutically acceptable carrier. Topical formulations for administration to the skin can include creams, ointments, and gels, for example, and can be prepared using oleaginous or water-soluble ointment bases, as is well known to those in the art. For example, these formulations may include vegetable oils, animal fats, and more preferably, semisolid hydrocarbons obtained from petroleum. Particular components used may include white ointment, yellow ointment, cetyl esters wax, oleic acid, olive oil, paraffin, petrolatum, white petrolatum, spermaceti, starch glycerite, white wax, yellow wax, lanolin, anhydrous lanolin, and glyceryl monostearate. Various water-soluble ointment bases may also be used including, for example, glycol ethers and derivatives, polyethylene glycols, polyoxyl 40 stearate, and polysorbates.

In some aspects of the invention, the PPARγ agonist, RXR agonist, and optionally LXR agonist described above find use in the treatment of Alzheimer's disease, as well as diseases and conditions with inflammatory components, including, but not limited to, stroke, ischemic damage to the nervous system, neural trauma (e.g., percussive brain damage, spinal cord injury, and traumatic damage to the nervous system), multiple sclerosis and other immune-mediated neuropathies (e.g., Guillain-Barre syndrome and its variants, acute motor axonal neuropathy, acute inflammatory demyelinating polyneuropathy, and Fisher Syndrome), HIV/AIDs dementia complex, and bacterial, parasitic, fungal, and viral meningitis and encephalitis.

Experiments conducted during development of the present invention demonstrate that RXR agonists stimulate the proteolytic degradation of Aβ by astrocytes, reduce pathology in an animal model of Alzheimer's Disease, reduce plaque burden in an animal model of Alzheimer's Disease, reduce Aβ in the brains in an animal model of Alzheimer's Disease, and reduce inflammation in an animal model of Alzheimer's Disease. Moreover, RXR agonists administered to a subject can inhibit the heterodimer partners to RXR, LXR and PPARγ and reduce the effects of RXR activation to promote intracellular Aβ degradation. Thus, the present invention provides methods and compositions for attenuating the progressive neurodegenerative processes in Alzheimer's disease and other diseases and conditions with an inflammatory component. However, it is not intended that the present invention be limited to any particular mechanism. Indeed, an understanding of the mechanisms is not necessary in order to practice the present invention.

In another aspect of the present invention, a variety of dermatological disorders can be treated by topically administering at least one PPARγ agonist, RXR agonist, and optionally LXR agonist or derivative thereof to a subject. A dermatological disorder can include any disorder of skin, hair or glands. A dermatological disorder can be manifest in the form of visible lesions, pre-emergent lesions, pain, sensitivity to touch, irritation, inflammation, or the like. Dermatological disorders can also include disorders of the cutaneous and pilosebaceous unit or the process of keratogenesis. For example, a dermatological disorder can be a disorder of the epidermis, dermis, subcutaneous layer, or combination thereof within and surrounding a pilosebaceous unit. Examples of dermatological disorders can include, but are not limited to, acne, alopecia, psoriasis, seborrhea, ingrown hairs and pseudofolliculitis barbae, hyperpigmented skin, cutaneous infections, lichen planus, Graham Little Syndrome, periorificial dermatitis, rosacea, hidradenitis suppurativa, dissecting cellulitis, systemic lupus erythematosus, discoid lupus erythematosus, and the like.

In another aspect of the present invention, at least one primary Cicatricial alopecia (CA) can be treated by topically administering at least one PPARγ agonist, RXR agonist, and optionally LXR agonist or derivative thereof to a subject. In general, CAs can be classified as lymphocytic, neutrophilic, and combinations thereof (i.e., “mixed”). Examples of lymphocytic CAs include lichen planopilaris, frontal fibrosing alopecia, chronic cutaneous lupus, erythematosus, pseudopelade, central centrifugal alopecia, alopecia mucinosa, and keratosis follicularis spinulosadecalvans. Examples of neutrophilic CAs include folliculitis decalvans, tufted folliculitis, and dissecting cellulitis. Examples of mixed CAs include follicullitis keloidalis and erosive dermatosis.

In an example of the present invention, a pharmaceutical composition comprising a thiazolidinedione, such as rosiglitazone and/or pioglitazone, and Bexarotene can be topically administered to treat a subject having a primary CA, such as LPP. A topical formulation comprising a thiazolidinedione and Bexarotene may be prepared in a gel or liquid, for example, and then administered to at least one region of the subject affected by LPP. For example The topical formulation may be administered to a portion of the subject's scalp exhibiting shiny, flat-topped bumps having an angular shape and a reddish-purplish color,

Administering the topical formulation to the affected region may inhibit or decrease peroxisome loss in at least one cell, such as in a sebaceous stem cell, by increasing expression of the PEX genes and/or genes associated with lipid β-oxidation and desaturation. This, in turn, may decrease or inhibit lipid accumulation in the pilosebaceous unit and thereby channel the lipid stores to increase β-oxidation and abrogate the deleterious effects of lipid overload, i.e., inflammation, loss of hair follicles, and fibrosis.

The following example included to demonstrate an embodiment of the invention. It should be appreciated by those of skill in the art that the techniques disclosed in the examples, which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLES

PPARγ and LXRs act in concert to regulate lipid metabolism and ApoE expression (FIG. 1). In brief, PPARγ acts as a physiological fatty acid sensor and upon dietary intake of fatty acids, they and their immediate metabolites bind to and activate PPARγ (FIG. 1). PPARγ activation then results in the stimulation of expression of enzymes of lipid metabolism, including induction of LXRα. Similarly, LXRs act as whole body cholesterol sensors and dietary cholesterol intake leads to the activation of the receptors and induction of a number of genes subserving cholesterol trafficking, metabolism and disposal. In addition, LXR activation results in induction of PPARγ, resulting in a feed-forward mechanism through which, the combined actions of these receptors are responsible for catabolism and storage of dietary lipids

In the brain, the primary RXR partners are LXR and PPARγ and their metabolic actions are similar to those observed in the periphery. Importantly, RXR agonists, acting alone, are sufficient to stimulate the transcriptional activity of the LXR and PPARγ heterodimers. However, the actions of RXR in the brain have not been extensively examined. It is important to point out that the RAR class of retinoic acid receptors also heterodimerize with RXR, but are termed ‘nonpermissive’ as they do not respond to RXR ligation. RARs bind all-trans retinoic acid, while RXRs do not.

The retinoid LGD1069 (Bexarotene, TARGRETIN) is the only FDA approved RXR agonist. Bexarotene is a highly selective retinoid X receptor (RXR) agonist developed for the treatment of cutaneous T-cell lymphoma and has recently been investigated in the treatment of psoriasis and breast cancer. Bexarotene has been shown to induce the expression of the LXR target genes, ABCA1 and ABCG1 in a murine model of mixed dyslipidemis. Clinically, Bexarotene has a good safety profile and has been used over extended periods in humans without significant side effects.

We found that (a) the ligation of RXR is as effective as either of the PPARγ and LXR agonists in stimulating the expression of their target genes and promoting Aβ degradation and (b) the RXR agonist results in positively cooperative effects whereby the effective dose to elicit the responses of PPARγ and LXR agonists are reduced. We also determined that RXR agonists, alone, or in combination with LXR and PPARγ agonists reduced plaque burden and alter cognition in a murine model of AD. These results are of potential therapeutic importance due to (1) the availability of FDA approved RXR agonists that could be used as monotherapy for AD, and (2) ongoing clinical trials of thiazolidinedione PPARγ agonists which have limited blood brain barrier (BBB) permeability and whose actions can be enhanced by combined treatment with RXR agonists.

### Example 1

### RXR Activation Drives the Expression LXR Target Genes

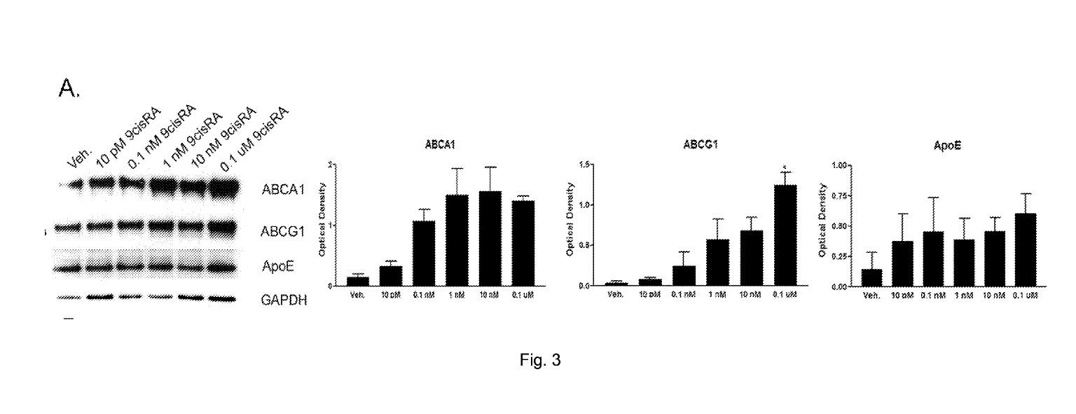

To determine if RXR activation regulates the expression of LXR target genes, we treated primary murine microglia with increasing doses of Bexarotene for 24 hours. We found that the RXR agonist treatment drives the expression of ABCA1, ABCG1, and ApoE (FIG. 3).

### Example 2

### RXR Activation Enhances ApoE Lipidation Status

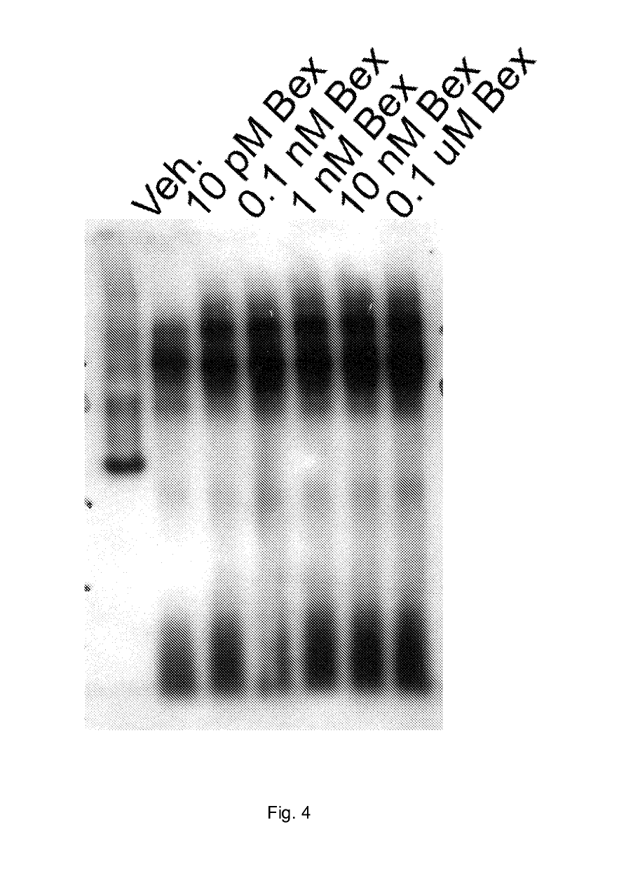

To determine if RXR ligation enhances the lipidation status of ApoE, confluent primary murine astrocytes were treated with increasing doses of Bexarotene for 48 hours. Astrocyte conditioned media was collected and assessed by native gel electrophoresis. We found that Bexarotene increases the lipidation status of ApoE, thus increasing the size of the ApoE particles. (FIG. 4).

### Example 3

### RXR Activation Stimulates the Proteolytic Degradation of Aβ by Microglia

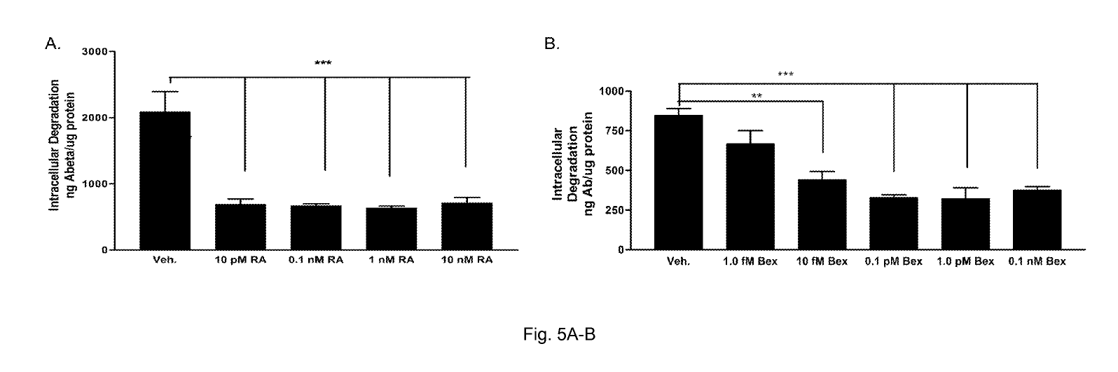

Given the ability of RXR activation to drive LXR target gene expression, we predicted that agonist treatment should promote the proteolytic degradation of Aβ by microglia. We found 9cisRA (FIG. 5A) and Bexarotene (FIG. 5B) treatment resulted in a dose-dependent reduction in intracellular Aβ levels. Aβ uptake was unaffected by either drug treatment.

### Example 4

### Oral RXR Agonist Treatment Regulates Gene Expression in the Brain

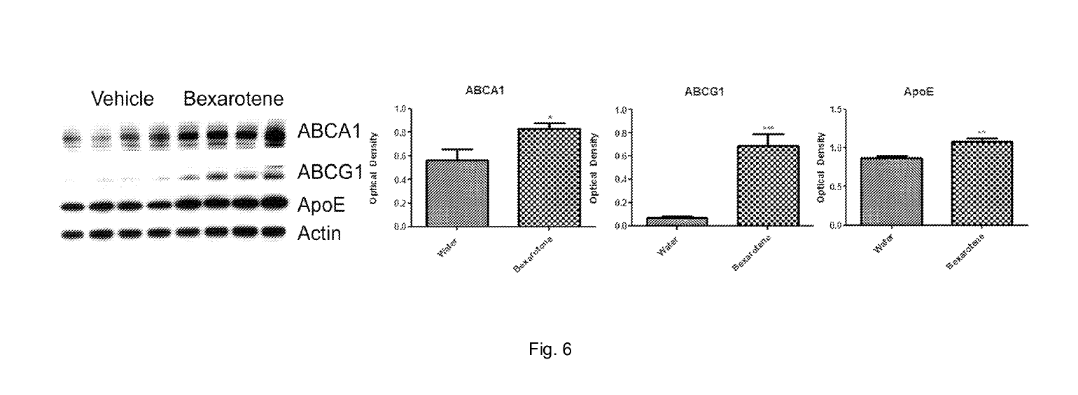

To verify that the RXR agonist, Bexarotene alters gene expression in the brain, we orally gavaged 6 month old mice with 100 mg/kg Bexarotene (n=4) or vehicle (water) (n=4 per group) for 7 days. We found drug treated animals exhibited elevated levels of ABCA1, ABCG1 and ApoE (FIG. 6), demonstrating that Bexarotene can directly regulate LXR target gene expression in the brain.

### Example 5

### RXR Agonist Treatment Reduces Aβ in the Brains of an AD Mouse Model

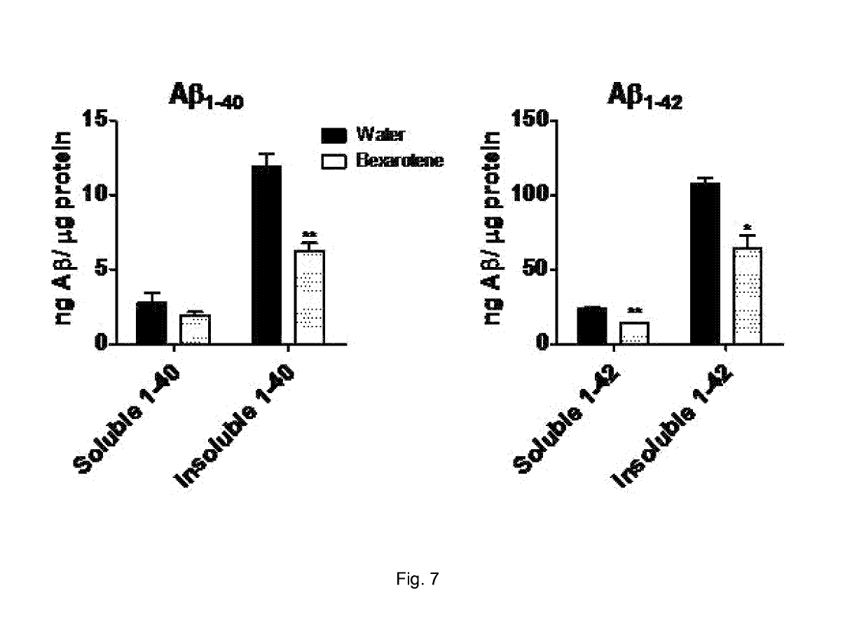

To determine if RXR ligation by Bexarotene alters the concentration of Aβ in the brains of transgenic mice that harbor both mutations in APP and PS1 (Borchelt animal model), ELISAs were performed on diethylamine (DEA) and formic acid (FA) extractions from brain homogenates. Both Aβ1-40 and Aβ1-42 were assessed on both soluble (DEA) and insoluble (FA) fractions. We found that a 7 day drug treatment of 6 month Borchelt animals reduces both soluble and insoluble Aβ fractions. (FIG. 7). We have increased the number of animals to at least an n of 4 per group.

### Example 6

### RXR Treatment Reduces Pathology in an Animal Model of Alzheimer's Disease

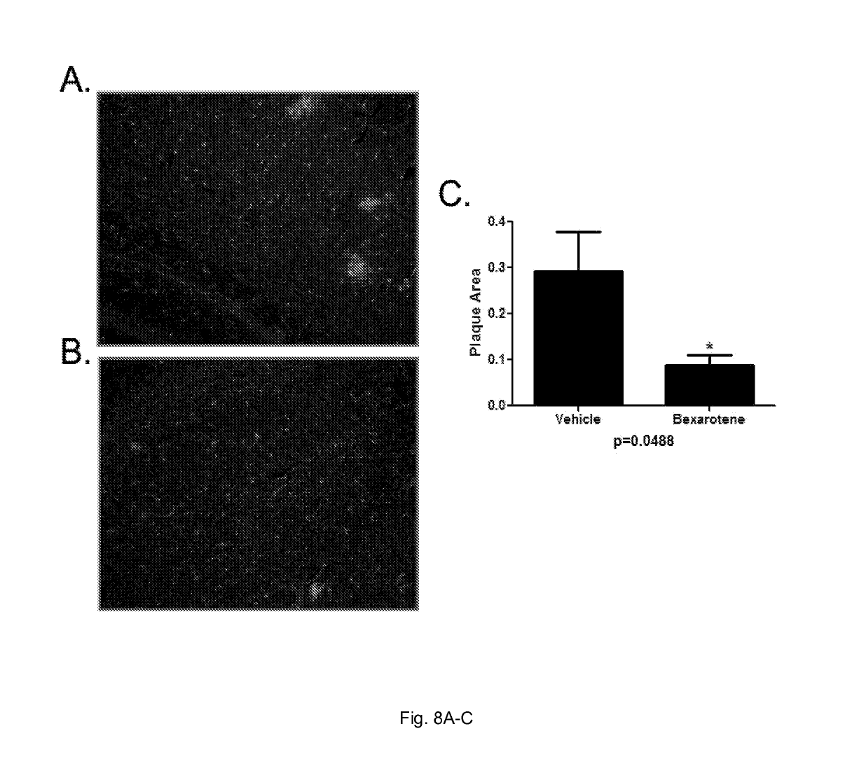

To determine whether treatment of Bexarotene reduces signs of AD pathology, we treated 6 month old AD mice for 7 days with Bexarotene by oral gavage (100 mg/kg/day in water). We extracted the brains of the mice, and assessed by immunohistochemistry plaque pathology (6E10). AD mice treated with Bexarotene show about 62% reduction in plaque burden in comparison to those not treated with Bexarotene (FIG. 8).

### Example 7

### RXR Treatment Improves Contextual Fear Conditioning Behavior in an AD Animal Model

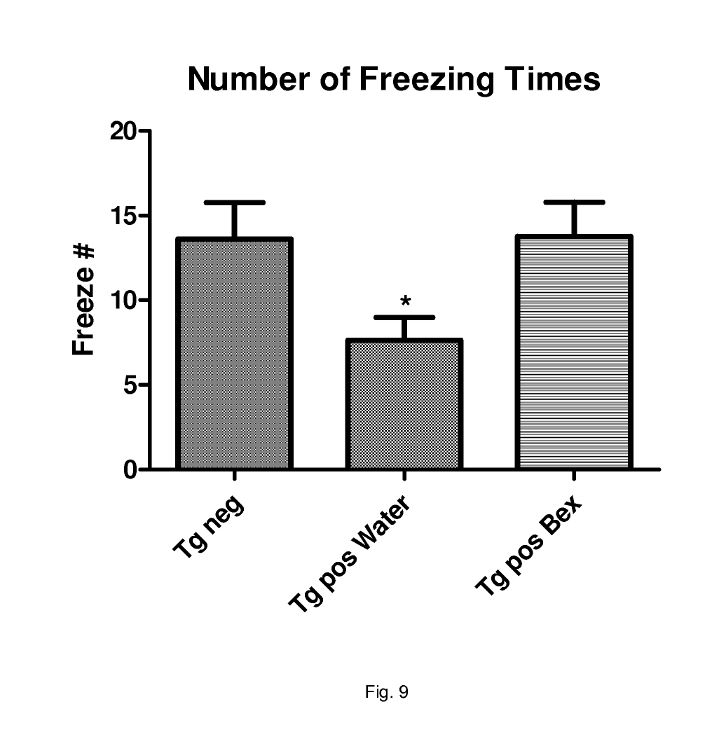

To determine whether treatment with Bexarotene improves the behavioral deficit found in the AD animal model, we orally gavaged 6 month old AD model mice for 7 days on Bexarotene (100 mg/kg/day in water) (n=8). We then assessed contextual fear conditioning behavior, an accepted behavioral test for Alzheimers's Disease study, and found that Bexarotene significantly improves behavior r (FIG. 9).

### Example 8

### RXR Activation Drives LXR Target Gene Expression in Astrocytes

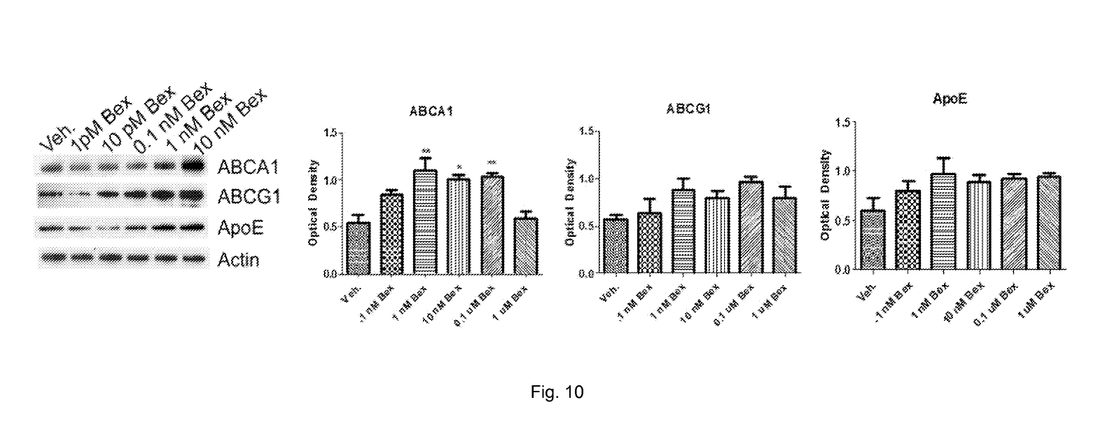

To determine if the effects of RXR activation can effect astrocytes as well as microglia, we treated primary murine astrocytes with increasing doses of Bexarotene for 24 hours. We found that RXR agonist treatment drives the expression of ABCA1, ABCG1 and ApoE (FIG. 10).

### Example 9

### RXR Activation Drives PPARγ Genes

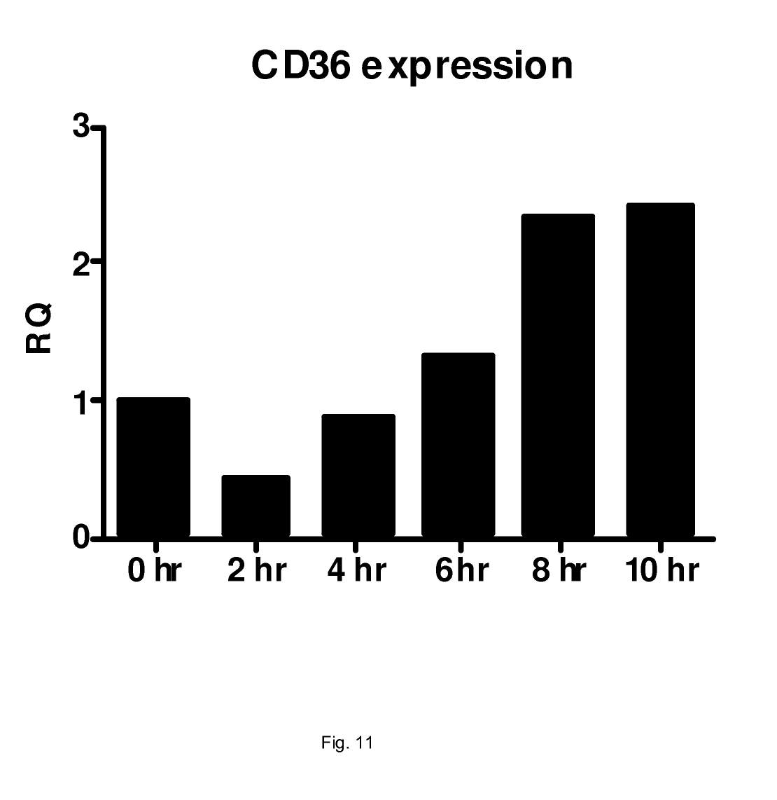

In order to confirm that RXR activation not only drives LXR target genes, but also PPARγ target genes, we treated confluent primary murine astrocytes with 10 nM Bexarotene in a time course. We found that RXR activation drives CD36, a PPARγ regulated gene, expression by qRT-PCR (FIG. 11).

### Example 10

### RXR Activation Stimulates the Proteolytic Degradation of Aβ by Astrocytes

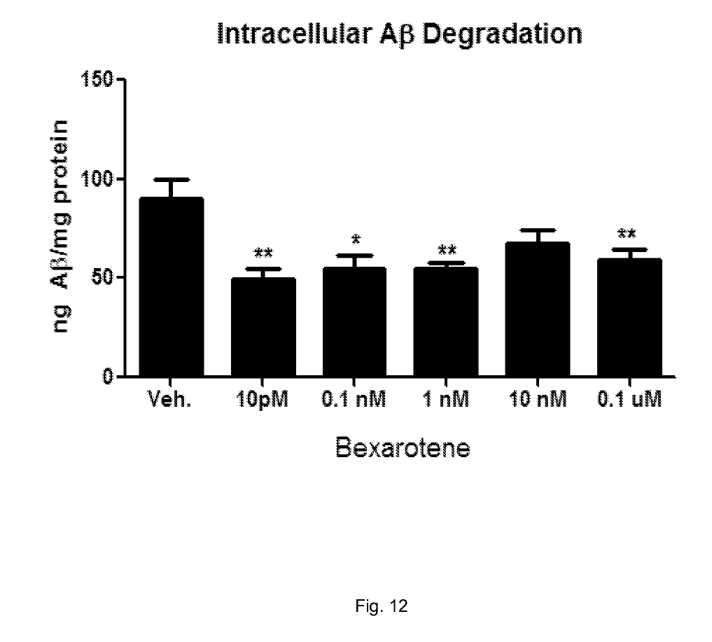

As shown above, astrocytes can drive the expression of LXR target genes after RXR activation, we predicted that agonist treatment should also promote the intracellular degradation of Aβ by astrocytes. We found that Bexarotene (FIG. 12) treatment resulted in a dose dependent reduction in intracellular Aβ levels. Aβ uptake was not affected by drug treatments (data not shown).

### Example 11

### ApoE is Necessary to Promote Intracellular Degradation by Both Murine Microglia and Astrocytes

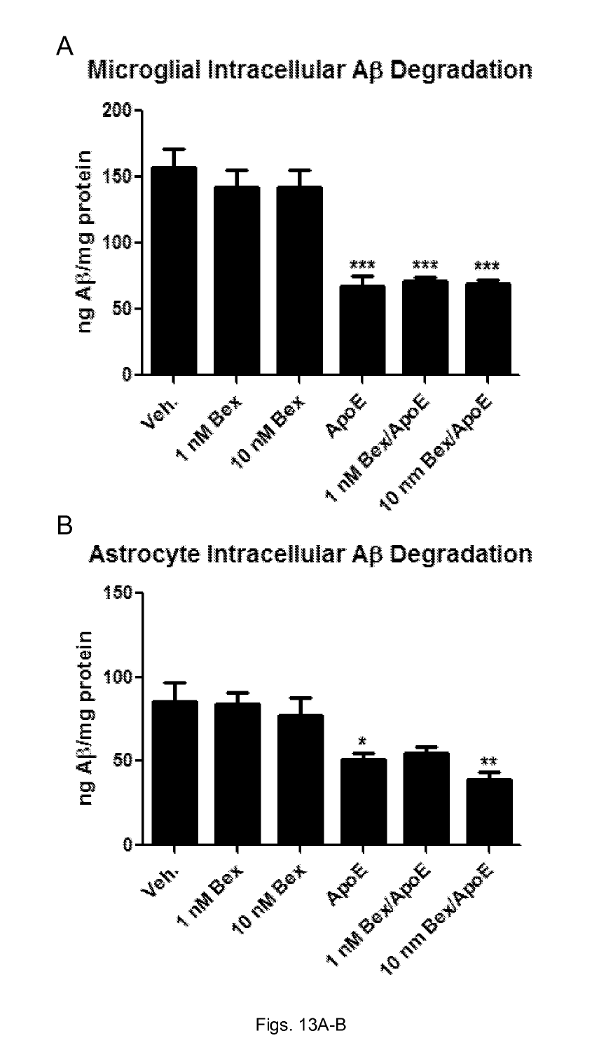

To determine if the ability of RXR activation to degrade Aβ is ApoE dependent, we used ApoE knock out microglia (A) or astrocytes (B) in the presence of Bexarotene. Bexarotene has no effect without the presence of ApoE, however, with the addition of exogenous ApoE, the effect of intracellular Aβ degradation returns (FIG. 13).

### Example 12

### Inhibiting the Heterodimer Partners to RXR, LXR and PPARγ, Reduces the Effects of RXR Activation to Promote Intracellular Aβ Degradation

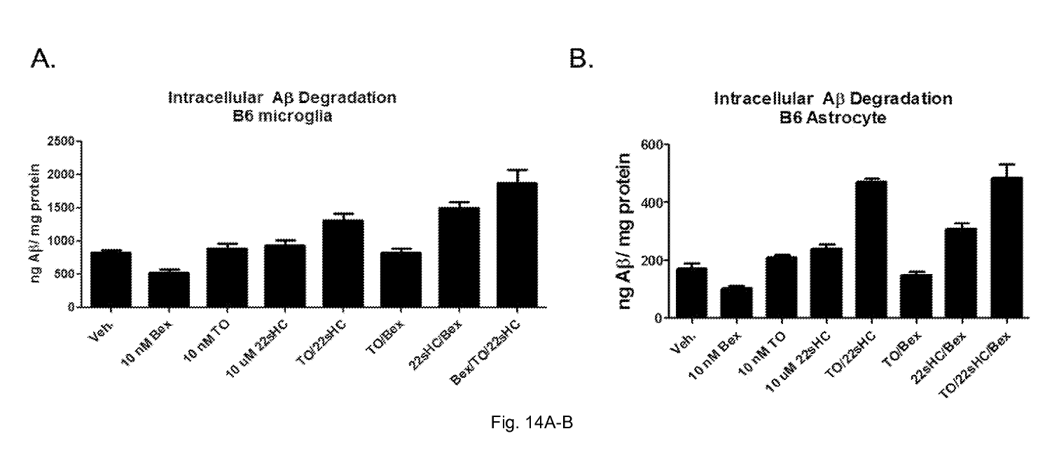

To determine which heterodimer partners are involved in RXR activation driven Aβ intracellular degradation, we inhibited PPARγ and LXR by competitive inhibitors, TO and 22-s-Hydroxycholesterol, respectively. Aβ degradation mediated by either microglia (A) or astrocytes (B) is inhibited with either inhibitor to PPAR or LXR. Additionally, a co-treatment with both inhibitors reduces Aβ degradation further (FIG. 14).

### Example 13

### RXR Activation Reduces Inflammation in an Animal Model of AD

To determine if activating RXR would reduce inflammation, we treated AD mouse models with Bexarotene (100 mg/kg/day in water) for 7 days and analyzed a marker for inflammation, Glial Fibrially Acidic Protein (GFAP). As mentioned above, we extracted the brains of the mice, and assessed by immunohistochemistryg GFAP expression. AD mice treated with Bexarotene show significantly reduced GFAP expression (FIG. 15).

### Example 14

### RXR Activation Induced Microglia to Take Up Aβ In Vivo

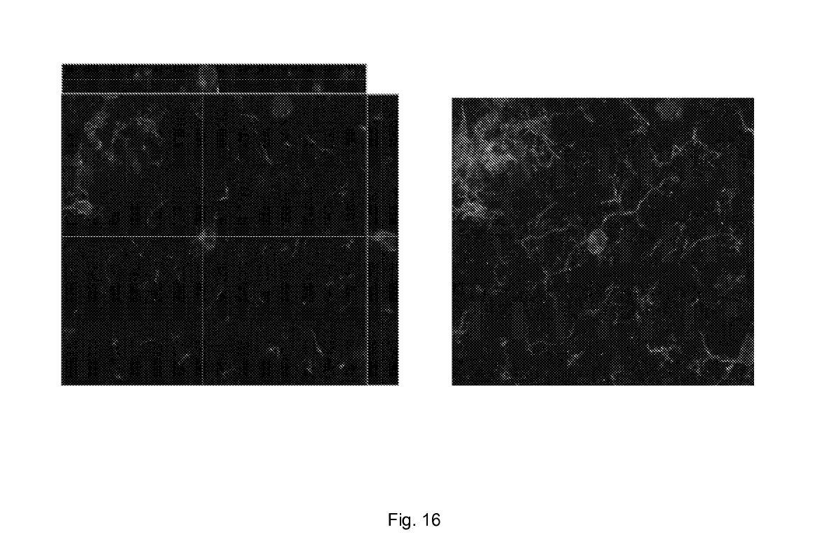

In order to determine if microglia are capable of taking up Aβ, we used confocal microscopy to show Aβ peptides within microglia, the brain's macrophage. We analyzed cryostat sections of transgenic, AD mouse models, with 6E10 and a marker for microglia, Iba1 treated with Bexarotene. Microglia in the brains of Bexarotene treated animals can take up Aβ in vivo (FIG. 16).

From the above description of the invention, those skilled in the art will perceive improvements, changes and modifications. Such improvements, changes and modifications within the skill of the art are intended to be covered by the appended claims. All references, publications, and patents cited in the present application are herein incorporated by reference in their entirety.

1-36. (canceled)

37: A method of treating a neurodegenerative disorder or a neural condition with an inflammatory component in a subject, comprising:
administering to the subject a therapeutically effective amount of at least one RXR agonist.

38: The method of claim 37, the neurodegenerative disorder selected from the group consisting of Alzheimer's disease, Parkinson's disease, and Huntington's disease.

39: The method of claim 37, the neural condition with an inflammatory component selected from the group consisting of central nervous system injuries, stroke, ischemic damage to the nervous system, neural trauma, multiple sclerosis, immune-mediated neuropathies, HIV/AIDs dementia complex, meningitis, and encephalitis.

40: The method of claim 37, the RXR agonist comprising Bexarotene.

41: The method of claim 37 further comprising administering a PPARγ agonist in combination with the RXR agonist.

42: The method of claim 41, the PPARγ agonist comprising a thiazolidinedione or a derivative thereof.

43: The method of claim 42, the PPARγ agonist comprising at least one compound or a pharmaceutically acceptable salt thereof selected from the group consisting of: (+)-5[[4(3,4-dihydro-6-hydroxy-2,5,7,8-tetramethyl-2H-1-benzopyran-2-yl)methoxy]phenyl]methyl]-2,4thiazolidinedione; 5-[4-[2-(5-ethylpyridin-2-yl)ethoxyl]benzyl]thiazolidine-2,4-dione; 5-[4-[(1-methylcyclohexyl)methoxy]benzyl]thiazolidine-2,4-dione; (ciglitazone); 4-(2-naphthylmethyl)-1,2,3,5-oxathiadiazole-2-oxide; 5-[4-[2-[(N-(benzoxazol-2-yl)-N-methylamino]ethoxy]benzyl]-5-methlthiazolidine-2,4-dione; 5-[4-[2-[2,4-dioxo-5-phenylthiazolidine-3-yl)ethoxy]benzyl]thiazolidine-2,4-dione; 5-[4-[2-[(N-methyl-N-(phenoxycarbonyl)amino]ethoxy]benzyl]thiazolidine-2,4-dione; 5-[4-[2-phenoxyethoxy)benzyl]thiazolidine-2,4-dione; 5-[4-[2-(4-chorophenyl)ethylsulfonyl]benzyl]thiazolidine-2,4-dione; 5-[4-[3-(5-methyl-2-phenyloxazol-4-yl)propionyl]benzyl]thiazolidine-2,4-dione; 5-[[4-(3-hydroxy-1-methylcyclohexyl)methoxy]benzyl]thiazolidine-2,4-dione; 5-[4-[2-(5-methyl-2-phenyloxazol-4-yl)ethoxyl]benzyl]thiazolidine-2,4-dione; 5-[(2-benzyl-2,3-dihydrobenzopyran)-5-ylmethyl]thiazolidine-2,4-dione; 5-[[2-(2-naphthylmethyl)benzoxazol]-5-ylmethyl]thiazolidine-2,4-dione; 5-[4-[2-(3-phenylureido)ethoxyl]benzyl]thiazolidine-2,4-dione; 5-[4-[2-(N-benzoxazol-2-yl)-N-metholamino]ethoxy]benzyl]thiazolidine-2,4-dione; 5-[4-[3-(5-methyl-2-phenyloxazol-4-yl)propionyl]benzyl]thiazolidine-2,4-dione; 5-[2-(5-methyl-2-phenyloxazol-4-ylmethyl)benzofuran-5-ylmethyl]oxazolidine-2,4-dione; 5-[4-[2-(N-methyl-N-(2-pyridyl)amino]ethoxy]benzyl]thiazolidine-2,4-dione; and 5-[4-[2-(N-(benzoxazol-2-yl)-N-methylamino]ethoxy]benzyl]oxazolidine-2,4-dione.

44: The method of claim 41, further comprising administering a LXR agonist to the subject.

45: A method of treating a neurodegenerative disorder in a subject, comprising:
administering to the subject therapeutically effective amounts of at least one RXR agonist and at least one PPARγ agonist.

46: The method of claim 45, the neurodegenerative disorder selected from the group consisting of Alzheimer's disease, Parkinson's disease, and Huntington's disease.

47: The method of claim 45, the RXR agonist comprising Bexarotene.

48: The method of claim 45, the PPARγ agonist comprising a thiazolidinedione or a derivative thereof.

49: The method of claim 48, the PPARγ agonist comprising at least one compound or a pharmaceutically acceptable salt thereof selected from the group consisting of: (+)-5[[4(3,4-dihydro-6-hydroxy-2,5,7,8-tetramethyl-2H-1-benzopyran-2-yl)methoxy]phenyl]methyl]-2,4thiazolidinedione; 5-[4-[2-(5-ethylpyridin-2-yl)ethoxyl]benzyl]thiazolidine-2,4-dione; 5-[4(1-methylcyclohexyl)methoxy]benzyl]thiazolidine-2,4-dione; (ciglitazone); 4-(2-naphthylmethyl)-1,2,3,5-oxathiadiazole-2-oxide; 5-[4-[2-[(N-(benzoxazol-2-yl)-N-methylamino]ethoxy]benzyl]-5-methlthiazolidine-2,4-dione; 5-[4-[2-[2,4-dioxo-5-phenylthiazolidine-3-yl)ethoxy]benzyl]thiazolidine-2,4-dione; 5-[4-[2-[(N-methyl-N-(phenoxycarbonyl)amino]ethoxy]benzyl]thiazolidine-2,4-dione; 5-[4-[2-phenoxyethoxy)benzyl]thiazolidine-2,4-dione; 5-[4-[2-(4-chorophenyl)ethylsulfonyl]benzyl]thiazolidine-2,4-dione; 5-[4-[3-(5-methyl-2-phenyloxazol-4-yl)propionyl]benzyl]thiazolidine-2,4-dione; 5-[[4-(3-hydroxy-1-methylcyclohexyl)methoxy]benzyl]thiazolidine-2,4-dione; 5-[4-[2-(5-methyl-2-phenyloxazol-4-yl)ethoxyl]benzyl]thiazolidine-2,4-dione; 5-[(2-benzyl-2,3-dihydrobenzopyran)-5-ylmethyl]thiazolidine-2,4-dione; 5-[[2-(2-naphthylmethyl)benzoxazol]-5-ylmethyl]thiazolidine-2,4-dione; 5-[4-[2-(3-phenylureido)ethoxyl]benzyl]thiazolidine-2,4-dione; 5-[4-[2-(N-benzoxazol-2-yl)-N-metholamino]ethoxy]benzyl]thiazolidine-2,4-dione; 5-[4-[3-(5-methyl-2-phenyloxazol-4-yl)propionyl]benzyl]thiazolidine-2,4-dione; 5-[2-(5-methyl-2-phenyloxazol-4-ylmethyl)benzofuran-5-ylmethyl]oxazolidine-2,4-dione; 5-[4-[2-(N-methyl-N-(2-pyridyl)amino]ethoxy]benzyl]thiazolidine-2,4-dione; and 5-[4-[2-(N-(benzoxazol-2-yl)-N-methylamino]ethoxy]benzyl]oxazolidine-2,4-dione.

50: The method of claim 45, further comprising administering a LXR agonist to the subject.

51: A method of treating a neural condition with an inflammatory component in a subject, comprising:
administering to the subject therapeutically effective amounts of at least one RXR agonist and at least one PPARγ agonist.

52: The method of claim 51, the neural condition with an inflammatory component selected from the group consisting of central nervous system injuries, stroke, ischemic damage to the nervous system, neural trauma, multiple sclerosis, immune-mediated neuropathies, HIV/AIDs dementia complex, meningitis, and encephalitis.

53: The method of claim 51, the RXR agonist comprising Bexarotene.

54. The method of claim 51, the PPARγ agonist comprising a thiazolidinedione or a derivative thereof.

55. The method of claim 54, the PPARγ agonist comprising at least one compound or a pharmaceutically acceptable salt thereof selected from the group consisting of: (+)-5[[4(3,4-dihydro-6-hydroxy-2,5,7,8-tetramethyl-2H-1-benzopyran-2-yl)methoxy]phenyl]methyl]-2,4thiazolidinedione; 5-[4-[2-(5-ethylpyridin-2-yl)ethoxyl]benzyl]thiazolidine-2,4-dione; 5-[4-[(1-methylcyclohexyl)methoxy]benzyl]thiazolidine-2,4-dione; (ciglitazone); 4-(2-naphthylmethyl)-1,2,3,5-oxathiadiazole-2-oxide; 5-[4-[2-[(N-(benzoxazol-2-yl)-N-methylamino]ethoxy]benzyl]-5-methlthiazolidine-2,4-dione; 5-[4-[2-[2,4dioxo-5-phenylthiazolidine-3-yl)ethoxy]benzyl]thiazolidine-2,4-dione; 5-[4-[2-[(N-methyl-N-(phenoxycarbonyl)amino]ethoxy]benzyl]thiazolidine-2,4-dione; 5-[4-[2-phenoxyethoxy)benzyl]thiazolidine-2,4-dione; 5-[4-[2-(4-chorophenyl)ethylsulfonyl]benzyl]thiazolidine-2,4-dione; 5-[4-[3-(5-methyl-2-phenyloxazol-4-yl)propionyl]benzyl]thiazolidine-2,4-dione; 5-[[4-(3-hydroxy-1-methylcyclohexyl)methoxy]benzyl]thiazolidine-2,4-dione; 5-[4-[2-(5-methyl-2-phenyloxazol-4-yl)ethoxyl]benzyl]thiazolidine-2,4-dione; 5-[(2-benzyl-2,3-dihydrobenzopyran)-5-ylmethyl]thiazolidine-2,4-dione; 5-[[2-(2-naphthylmethyl)benzoxazol]-5-ylmethyl]thiazolidine-2,4-dione; 5-[4-[2-(3-phenylureido)ethoxyl]benzyl]thiazolidine-2,4-dione; 5-[4-[2-(N-benzoxazol-2-yl)-N-metholamino]ethoxy]benzyl]thiazolidine-2,4-dione; 5-[4-[3-(5-methyl-2-phenyloxazol-4-yl)propionyl]benzyl]thiazolidine-2,4-dione; 5-[2-(5-methyl-2-phenyloxazol-4-ylmethyl)benzofuran-5-ylmethyl]oxazolidine-2,4-dione; 5-[4-[2-(N-methyl-N-(2-pyridyl)amino]ethoxy]benzyl]thiazolidine-2,4-dione; and 5-[4-[2-(N-(benzoxazol-2-yl)-N-methylamino]ethoxy]benzyl]oxazolidine-2,4-dione.

56: The method of claim 51, further comprising administering a LXR agonist to the subject.
